(19) United States
(12) Patent Application Publication
(10) Pub. No.: US 2023/0312673 A1
(43) Pub. Date: Oct. 5, 2023

(54) CHIMERIC RECEPTORS WITH DIVERSE CO-REGULATORY SEQUENCES

(71) Applicant: The Regents of the University of California, Oakland, CA (US)

(72) Inventors: Kole T. ROYBAL, San Francisco, CA (US); Daniel B. GOODMAN, San Francisco, CA (US); Camillia AZIMI, San Francisco, CA (US); Alexander MARSON, San Francisco, CA (US); Jeffrey A. BLUESTONE, San Francisco, CA (US)

(21) Appl. No.: 18/043,716

(22) PCT Filed: Sep. 1, 2021
(86) PCT No.: PCT/US2021/048697

§ 371 Date: Mar. 1, 2023

Related U.S. Application Data
(60) Provisional application No. 63/073,824, filed on Sep. 2, 2020.

(51) Int. Cl. C07K 14/725; C07K 14/705

(52) U.S. Cl. CPC C07K 14/7051; C07K 14/70521; C07K 14/70575; C07K 2317/622; C07K 2319/03

(57) ABSTRACT

This disclosure relates to chimeric receptors (CRs) and their signaling components for the regulation of an immune response. Also provided are nucleic acids encoding the disclosed CRs, recombinant immune cells expressing the same, and pharmaceutical compositions containing the disclosed nucleic acids and/or recombinant cells. Further provided are methods useful for modulating an activity of an immune cell, methods for modulating an immune response in an individual, as well as methods for treating a health condition in an individual in needed thereof

### CROSS-REFERENCE TO RELATED APPLICATIONS

The present application claims priority to U.S. Provisional Patent Application Ser. No. 63/073,824, filed on Sep. 2, 2020, the disclosure of which is incorporated by reference herein in its entirety, including any drawings.

### INCORPORATION OF THE SEQUENCE LISTING

The material in the accompanying Sequence Listing is hereby incorporated by reference into this application. The accompanying Sequence Listing text file, named “048536-677001WO_Sequence Listing_ST25.txt.” was created on Aug. 30, 2021 and is 92 KB.

### FIELD

This disclosure relates to chimeric receptors (CRs) and their signaling components for the regulation of an immune response, as well as methods for producing and using the same.

### BACKGROUND

New synthetic cellular receptors (including CARs, SynNotch, etc.) are ushering in many new treatment options for previously intractable cancers, and present promising therapeutic strategies for other diseases. However, the enormous space of possible synthetic receptor designs has only begun to be explored. For example, while CAR T cell therapy has had an unprecedented impact for specific hematological malignancies, current CAR T cell therapies have had limited efficacy in solid tumors.

In order to identify new synthetic receptors which may have improved characteristics for various therapeutic purposes, for example, new receptors whose properties can be tuned to achieve desired activity and/or functionality, there remains a need for more efficient and high-throughput strategies. These new and enhanced synthetic receptors can be useful for the treatment of solid tumors or other indications for which current therapies have proven ineffective.

### SUMMARY

The present disclosure relates generally to improved compositions for adoptive cell therapy. In particular, the disclosure relates to chimeric receptors (CRs) and their signaling components for the regulation of an immune response, including CRs with properties that can be tuned to achieve desired functionality by ways of varying their signaling components and the arrangements of these components in the CRs. This is because many existing recombinant chimeric receptors (often referred to as Pt generation chimeric receptors) can only function as an “on/off” switch with no or little mean to allow for fine-tuning their activity and/or functionality. In particular, some embodiments of the disclosure relate to a new class of chimeric receptors (CRs) engineered to include an intracellular signaling domain (SD) whose identity demonstrates an impact on effector function of immune cells expressing the CR. Accordingly, modifying the signaling component of a CR by introducing one or more endodomains significantly impacts CR-mediated signaling. In some embodiments of the disclosure, the CRs were designed herein to effect one or more immunomodulatory functions, such as co-stimulatory or inhibitory functions. Also provided herein are nucleic acids encoding the disclosed CRs, recombinant immune cells expressing the same, pharmaceutical compositions containing the disclosed nucleic acids and/or recombinant cells, and methods useful for modulating an activity of an immune cell, methods for modulating an immune response in an individual, as well as methods for treating a health condition in an individual in needed thereof.

In one aspect, provided herein are chimeric receptors including: (a) an extracellular antigen-binding domain capable of binding to a target antigen; (b) a transmembrane domain (TMD); and (c) an intracellular signal transduction domain including an intracellular signaling domain (SD) derived from a signaling molecule selected from the group consisting of 4-1BB, BAFF-R, BCMA, BTLA, CD2, CD200R, CD244, CD28, CD300a, CD300f, CD40, CD7, CD72, CD96, CRACC, CRTAM, CTLA4, CXADR, DC-SIGN, GITR, HAVCR2, ICOS, ILT2, ILT3, ILT4, KIR2DL1, KIR3DL1, KLRG1, LAG3, LAIR1, NKG2D, NKR-P1A, NTB-A, PD1, Siglec-3, TACI, TIGIT, TLT-1, and TNR8 (CD30).

Non-limiting exemplary embodiments of the disclosed chimeric receptors can include one or more of the following features. In some embodiments, the intracellular signal transduction domain includes a modulatory SD capable of mediating a co-stimulatory signal derived from a signaling molecule selected from the group consisting of BAFF-R, CD40, TACI, CD2, CD7, CD30, and NTB-A. In some embodiments, the modulatory SD is derived from: (a) BAFF-R and includes the amino acid sequence of SEQ ID NO: 24 or a variant thereof having at least about 80% sequence identity to SEQ ID NO: 24; (b) CD40 and includes the amino acid sequence of SEQ ID NO: 10 or a variant thereof having at least about 80% sequence identity to SEQ ID NO: 10; (c) TACI and includes the amino acid sequence of SEQ ID NO: 25 or a variant thereof having at least about 80% sequence identity to SEQ ID NO: 25; (d) CD2 and includes the amino acid sequence of SEQ ID NO: 19 or a variant thereof having at least about 80% sequence identity to SEQ ID NO: 19; (e) CD7 and includes the amino acid sequence of SEQ ID NO: 3 or a variant thereof having at least about 80% sequence identity to SEQ ID NO: 3; (f) CD30 and includes the amino acid sequence of SEQ ID NO: 21 or a variant thereof having at least about 80% sequence identity to SEQ ID NO: 21; or (g) NTB-A and includes the amino acid sequence of SEQ ID NO: 29 or a variant thereof having at least about 80% sequence identity to SEQ ID NO: 29.

In some embodiments, the intracellular signal transduction domain further includes an activation domain. In some embodiments, the activation domain includes one or more immunoreceptor tyrosine-based activation motifs (ITAMs). In some embodiments, the activation domain is 80, 85, 90, 95, 96, 97, 98, 99, or 100% identical to a CD3 activation domain. In some embodiments, the extracellular antigen-binding domain includes an antibody moiety capable of binding to the target antigen. In some embodiments, the antibody moiety is a scFv. In some embodiments, the target antigen is CD19, CD20, or MAGE. In some embodiments, the extracellular antigen-binding domain includes an extracellular domain derived from an inhibitory immune checkpoint molecule or a binding moiety capable of binding to a ligand of the inhibitory immune checkpoint molecule. In some embodiments, the intracellular signal transduction domain includes a modulatory SD capable of mediating an inhibitory signal derived from a signaling molecule selected from the group consisting of KLRG1, DC-SIGN, NKG2D, and NKR-P1A.

In some embodiments, the modulatory SD is derived from: (a) KLRG1 and includes the amino acid sequence of SEQ ID NO: 32 or a variant thereof having at least about 80% sequence identity to SEQ ID NO: 32; (b) DC-SIGN and includes the amino acid sequence of SEQ ID NO: 43 or a variant thereof having at least about 80% sequence identity to SEQ ID NO: 43; (c) NKG2D and includes the amino acid sequence of SEQ ID NO: 22 or a variant thereof having at least about 80% sequence identity to SEQ ID NO: 22; or (d) NKR-P1A and includes the amino acid sequence of SEQ ID NO: 33 or a variant thereof having at least about 80% sequence identity to SEQ ID NO: 33.

In some embodiments, the extracellular antigen-binding domain includes an extracellular domain derived from a stimulatory immune checkpoint molecule or a binding moiety capable of binding to a ligand of the stimulatory immune checkpoint molecule.

In one aspect, provided herein are recombinant nucleic acids including a nucleotide sequence encoding a chimeric receptor as disclosed herein. Non-limiting exemplary embodiments of the disclosed recombinant nucleic acids can include one or more of the following features. In some embodiments, nucleotide sequence encodes a chimeric receptor including: (a) an extracellular antigen-binding domain capable of binding to a target antigen; (b) a transmembrane domain; and (c) an intracellular signal transduction domain including an intracellular signaling domain (SD) derived from a signaling molecule selected from the group consisting of 4-1BB, BAFF-R, BCMA, BTLA, CD2, CD200R, CD244, CD28, CD300a, CD300f, CD40, CD7, CD72, CD96, CRACC, CRTAM, CTLA4, CXADR, DC-SIGN, GITR, HAVCR2, ICOS, ILT2, ILT3, ILT4, KIR2DL1, KIR3DL1, KLRG1, LAG3, LAIR1, NKG2D, NKR-P1A, NTB-A, PD1, Siglec-3, TACI, TIGIT, TLT-1, and TNR8 (CD30). In some embodiments, the nucleotide sequence is incorporated into an expression cassette or an expression vector. In some embodiments, the expression vector is a viral vector. In some embodiments, the viral vector is a lentiviral vector, an adenovirus vector, an adeno-associated virus vector, or a retroviral vector.

In another aspect, some embodiments of the disclosure relate to compositions including a recombinant nucleic acid as disclosed herein, wherein the composition is formulated for introducing the recombinant nucleic acid into a cell. In some embodiments, the composition is formulated as a lipid nanoparticle (LNP), liposome, or viral particle.

In another aspect, provided herein are recombinant immune cells including: (a) a chimeric receptor of the disclosure, and/or (b) a recombinant nucleic acid of the disclosure.

Non-limiting exemplary embodiments of the disclosed recombinant immune cells can include one or more of the following features. In some embodiments, the recombinant immune cell is a recombinant T cell. In some embodiments, the recombinant T cell is a recombinant CD4⁺ T cell or a recombinant CD8⁺ T cell. In some embodiments, the recombinant immune cell includes a chimeric receptor as disclosed herein and has one or more of the following properties: (a) enhanced proliferation in response to stimulation with the target antigen as compared to a corresponding cell that includes a corresponding receptor where the intracellular SD is derived from CD28 or 4-1BB; (b) enhanced expression of activation marker CD69 in response to stimulation with the target antigen as compared to a corresponding cell that includes a corresponding receptor where the intracellular SD is derived from CD28 or 4-1BB; (c) enhanced expression of IFNγ, TNFα, IL-2, and/or IL-4 in response to stimulation with the target antigen as compared to a corresponding cell that includes a corresponding receptor where the intracellular SD is derived from CD28 or 4-1BB; (d) enhanced resistance to exhaustion in response to stimulation with the target antigen as compared to a corresponding cell that includes a corresponding receptor where the intracellular SD is derived from CD28 or 4-1BB; and (e) enhanced killing of target cells expressing the target antigen as compared to a corresponding cell that includes a corresponding receptor where the intracellular SD is derived from CD28 or 4-1BB.

In some embodiments, the recombinant immune cell includes a chimeric receptor as disclosed herein and has one or more of the following properties: (a) reduced proliferation in response to stimulation with the target antigen as compared to a corresponding cell that includes a corresponding receptor where the intracellular SD is derived from an inhibitory signaling receptor selected from CTLA-4 and PD1; (b) reduced expression of activation marker CD69 in response to stimulation with the target antigen as compared to a corresponding cell that includes a corresponding receptor where the intracellular SD is derived from an inhibitory signaling receptor selected from CTLA-4 and PD1; (c) reduced expression of IFNγ, TNFα, and/or IL-2 in response to stimulation with the target antigen as compared to a corresponding cell that includes a corresponding receptor where the intracellular SD is derived from an inhibitory signaling receptor selected from CTLA-4 and PD1; (d) reduced resistance to exhaustion in response to stimulation with the target antigen as compared to a corresponding cell that includes a corresponding receptor where the intracellular SD is derived from an inhibitory signaling receptor selected from CTLA-4 and PD1; and (e) reduced killing of target cells expressing the target antigen as compared to a corresponding cell that includes a corresponding receptor where the intracellular SD is derived from an inhibitory signaling receptor selected from CTLA-4 and PD1.

In another aspect, provided herein are pharmaceutical compositions including a pharmaceutically acceptable carrier and one or more of the following: (a) a recombinant nucleic acid as disclosed herein; and (b) a recombinant immune cell as disclosed herein. Non-limiting exemplary embodiments of the disclosed pharmaceutical compositions can include one or more of the following features. In some embodiments, the pharmaceutical composition includes a recombinant nucleic acid of the disclosure. In some embodiments, the recombinant nucleic acid is encapsulated in an LNP, liposome, or viral particle.

In another aspect, provided herein are methods for modulating the activity of an immune cell, the methods include: (a) expressing a chimeric receptor of the disclosure in the immune cell; and (b) contacting the immune cell with the target antigen. In some embodiments, the contacting is carried out in vivo, ex vivo, or in vitro. In some embodiments, the immune cell includes a chimeric receptor of the disclosure, and the activity of the immune cell is increased following the contacting as compared to the immune cell prior to the contacting or a corresponding immune cell that does not express the chimeric receptor. In some embodiments, the immune cell includes a chimeric receptor of the disclosure and the activity of the immune cell is decreased following the contacting as compared to the immune cell prior to the contacting or a corresponding immune cell that does not express the chimeric receptor.

In yet other aspects, some embodiments of the disclosure relate to methods for modulating an immune response to a first antigen in an individual, the methods include administering to the individual an effective amount of recombinant immune cells as disclosed herein.

Non-limiting exemplary embodiments of the disclosed methods for modulating an immune response can include one or more of the following features. In some embodiments, the recombinant immune cells include a chimeric receptor of the disclosure, the chimeric receptor is capable of binding to the first antigen, and an immune response to the first antigen is stimulated. In some embodiments, the recombinant immune cells include a chimeric receptor of the disclosure, the chimeric receptor is capable of binding to the first antigen, and an immune response to the first antigen is inhibited. In some embodiments, the recombinant immune cells include: (a) a chimeric receptor of the disclosure, wherein the chimeric receptor is activated by binding to a repressor antigen; and (b) a second receptor capable of binding to the first antigen to stimulate an immune response to the first antigen. In some embodiments, the first antigen is present on a target cell to which an immune response is desired and the repressor antigen is present on a non-target cell to which an immune response is not desired. In some embodiments, the methods further include administering to the individual an effective amount of the repressor antigen such that an immune response to the first antigen is inhibited.

In another aspect, provided herein are methods for modulating an immune response to an antigen in an individual, the methods include administering to the individual an effective amount of a pharmaceutical composition as disclosed herein such that the recombinant nucleic acid is introduced into immune cells in the individual capable of mediating the immune response. In some embodiments, the pharmaceutical composition includes a chimeric receptor as disclosed herein, wherein the chimeric receptor is capable of binding to the antigen, and an immune response to the antigen is stimulated. In some embodiments, the pharmaceutical composition includes a chimeric receptor as disclosed herein, wherein the chimeric receptor is capable of binding to the antigen, and an immune response to the antigen is inhibited.

In yet another aspect, provided herein are methods for treating a health condition in an individual in need thereof, including administering to the individual an effective amount of recombinant immune cells of the disclosure, wherein the recombinant immune cells treat the health condition. In some embodiments, the recombinant immune cells include a chimeric receptor of the disclosure, and the health condition is characterized by a pathogenic cell expressing the target antigen of the chimeric receptor. In some embodiments, the recombinant immune cells include (a) a chimeric receptor of the disclosure, wherein the chimeric receptor is activated by binding to a repressor antigen; and (b) a second receptor capable of binding to a second antigen associated with the health condition to stimulate an immune response to the second antigen. In some embodiments, the second antigen is present on a pathogenic cell associated with the health condition and the repressor antigen is present on a non-pathogenic cell. In some embodiments, the method further includes administering to the individual an effective amount of the repressor antigen such that an adverse effect in the individual mediated by the recombinant immune cells is inhibited.

In yet another aspect, provided herein are methods for treating a health condition in an individual in need thereof, the methods including administering to the individual an effective amount of the pharmaceutical composition as disclosed herein such that the recombinant nucleic acid is introduced into immune cells in the individual to generate recombinant immune cells that treat the health condition. In some embodiments, the pharmaceutical composition includes a chimeric receptor of the disclosure, and the health condition is characterized by a pathogenic cell expressing the target antigen. In some embodiments, the pharmaceutical composition includes a chimeric receptor as disclosed herein, and the health condition is characterized by an adverse immune response to the target antigen.

In another aspect, provided herein are embodiments of a system or kit for modulating an activity of an immune cell, modulating an immune response in an individual, or treating a health condition in an individual in need thereof, wherein the system or kit includes one or more of the following: (a) a chimeric receptor of the disclosure; (b) a recombinant nucleic acid of the disclosure; (c) a recombinant immune cell of the disclosure; and (d) a pharmaceutical composition of the disclosure.

Non-limiting exemplary embodiments of the disclosed systems and kits can include one or more of the following features. In some embodiments, the system or kit includes a plurality of the chimeric receptors as disclosed herein, wherein the plurality of receptors includes at least 3, 4, 5, 6, 7, 8, 9, 10, 15, 20, 25, 30, 35, 40, 45, 48, or 50 different SDs. In some embodiments, the system or kit further includes a chimeric receptor as disclosed herein, wherein the intracellular SD is replaced with an intracellular SD derived from CD28 and/or 4-1BB. In some embodiments, the system or kit includes a plurality of the recombinant nucleic acids as disclosed herein, wherein the plurality of recombinant nucleic acids individually include nucleic acid sequences encoding one or more chimeric receptors of the disclosure. In some embodiments, the plurality of chimeric receptors encoded by the recombinant nucleic acids of the kit collectively include at least 3, 4, 5, 6, 7, 8, 9, 10, 15, 20, 25, 30, 35, 40, 45, 48, or 50 different SDs. In some embodiments, the system or kit includes a plurality of the recombinant immune cells as disclosed herein, wherein the plurality of recombinant immune cells include a plurality of receptors which collectively include at least 3, 4, 5, 6, 7, 8, 9, 10, 15, 20, 25, 30, 35, 40, 45, 48, or 50 different SDs. In some embodiments, the SD included in each of the plurality of chimeric receptor is different from the SDs included in other chimeric receptors.

In yet another aspect, some embodiments of the disclosure relate to chimeric receptors as disclosed herein, recombinant nucleic acids as disclose herein, immune cell as disclosed herein for use in the treatment of a health condition.

In yet another aspect, some embodiments of the disclosure relate to chimeric receptors as disclosed herein, recombinant nucleic acids as disclose herein, immune cell as disclosed herein for use in the manufacture of a medicament.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 depicts a schematic of CAR-T Cell engaging a target cell. T cells engineered to express a CAR (Chimeric Antigen Receptor) recognize the target antigen on the surface of the target cell via binding between the antigen and the CAR's N-terminal antigen-binding domain (e.g., a scFv). The antigen-binding domain is followed by a short hinge domain which can alter expression, dimerization, and binding. The CAR also contains a transmembrane domain (TMD), a costimulatory domain (whose modification in the library of the disclosure will be described in greater detail below), and an ITAM-containing C-terminal domain, which is usually the intracellular region of the CD3 zeta chain.

FIG. 2 depicts an overview of Flow-Seq measurement. In this figure, the pooled CAR library was subject to several different pooled assays as shown over the course of 24 days. An exemplary cell trace violet (CTV) proliferation assay is shown. After sorting the pooled library into bins based on CTV signal, the variable costimulatory region of the construct was amplified off of the genome and converted into approximate sorted cell counts. CD4 and CD8 T cell assays are shown below, with CARs ranked based on their average proliferation based on the pooled and binned CTV assay, with the most proliferative CAR costimulatory domains shown on the left side. CD4 T cell assays and CD8 T cell assays were performed on Day 3 and Day 4, respectively.

FIG. 3 depicts hierarchical clustering of CARs (y axis) vs Pooled Assays (x axis). Colors and values are the z-scores for each assay; CARs are clustered and ranked top to bottom from most proliferative to least. Z-scores are calculated by combining scaled values within each donor and replicate. Different CARs perform better at different timepoints (early vs late) and in CD4s vs CD8s (see, e.g., table/heatmap).

FIG. 4 depicts volcano plot of CAR performance across different assay times and T cell subsets. The leftmost bin in this figure was compared to the two rightmost bins to show estimated fold-change in abundance between the sorted populations, and associated with an adjusted p-value based on a count-adjusted negative binomial model. CAR costimulatory domains whose difference is significant in the assay across multiple donors are shown as larger points, while the benchmark costimulatory domains 4-1BB and CD28 are shown in shades of blue. Domains with extreme p-values are shown as triangles on the top edge of the plot.

FIG. 5 graphically summarizes the results of experiments comparing CD4 proliferation and CD8 T cell proliferation across the time points. Proliferation in CD4s and CD8s are compared, using the same analysis and features as described in the previous figure. The Y axis is the relative log2 ratio of CD8 to CD4 T cell proliferation, while the X axis shows mean relative proliferation of CD4 and CD8 T cells. Proliferation is generally stronger/more differential in CD8 T cells at early time points. At later time points, CARs with a subset of costimulatory domains (CD40, TNR8 (CD30), 4-1BB) expand preferentially in CD4 T cells while others (CD2, TACI, CD244) expand preferentially in CD8 T cells.

FIG. 6 graphically summarizes the results of experiments illustrating abundance change over time for the CARs. Y axis shows relative abundance change relative to baseline abundance in the library over time. Top (green) and bottom (pink) six CARs are shown, along with CD28 and 4-1BB control CARs shown in shades of blue.

FIG. 7 graphically summarizes the results of experiments to illustrate relative activation and cytokine secretion upon initial stimulation with target cells, showing pooled sorting-based analysis of activation and cytokine production via surface and intracellular staining. Open circles are from co-culture with target cells not expressing CD19 antigen, and closed circles from co-culture with target cells that do express CD19 antigen. CD28 strongly secretes IL-2, ILT2 is the only CAR to significantly secret IL-4, and BAFF-R and CD28 secrete significantly higher amounts of IFNγ.

FIG. 8 graphically summarizes the results of pooled experiments illustrating abundance change over time for the expanded set of CARs. Y axis shows relative abundance change relative to baseline abundance in the library over time. X axis shows number of days in culture with repeated stimulation via addition of target cells every three days.

FIG. 9 depicts a PCA plot of CAR performance across all assays and identification of 5 novel CARs for arrayed follow up. In subsequent arrayed screens, the focus was placed on the new receptors in greens and purples as well as a negative control receptor, in red.

FIG. 10 is a schematic of the arrayed assays on a single timeline. This process was performed with two donors. All assays are performed separately on CD8s and CD4s, but only cytokines were assayed for CD4s.

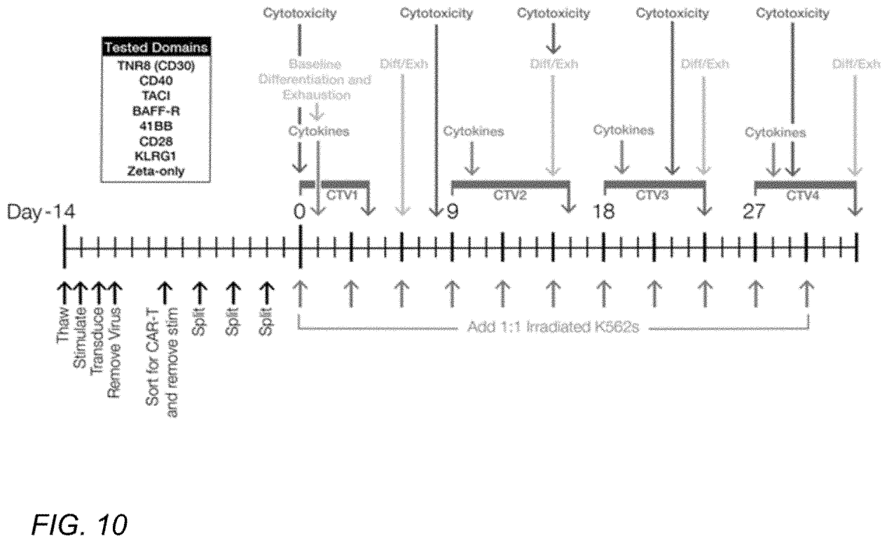

FIG. 11 graphically summarizes the results of experiments performed to illustrate CD8 exhaustion. Number of different exhaustion markers (CD39, LAG3, TIM3, PD1) present per cell at different time points is shown. CD40 and BAFF-R express less of these markers overall, and a lower proportion of the cells have three or more markers present at later time points.

FIG. 12 graphically summarizes the results of experiments performed to illustrate CD4 exhaustion. Number of different exhaustion markers (CD39, LAG3, TIM3, PD1) present per cell at different time points is shown. CD28 and BAFF-R express less of these markers overall, and a lower proportion of the cells have three or more markers present later time points.

FIG. 13 graphically summarizes the results of experiments performed to illustrate that differential costimulatory domains alter proliferation dynamics. In these experiments, primary human CD4 and CD8 T cells were lentivirally transduced with each CAR, sorted for purity, and stimulated with CD19+or CD19- irradiated K562 tumor cells every three days over a period of 33 days. Four (4) Cell Trace Violet (CTV) stains were assayed using flow cytometry at various times shown in the schematic at the top, and a representative donor is shown at Day 4 (CTV1), and Day 24 (CTV3), comparing CAR proliferation (lower is more proliferative).

FIG. 14 graphically depicts the results of experiments performed to illustrate that differential costimulatory domains alter proliferation dynamics. In these experiments, four (4) CTV stains were assayed using flow cytometry at various times as shown in the schematic at the top of the previous figure. In these experiments, a single donor (Donor 1) is shown for all 4 time points across all 8 assayed CARs in CD4 and CD8 T cells.

FIG. 15 graphically depicts the results of additional experiments performed to illustrate that differential costimulatory domains alter proliferation dynamics. Similarly to experiments described in FIG. 14, four (4) CTV stains were assayed using flow cytometry at various times as shown in the schematic at the top of FIG. 13. In these experiments, a single donor (Donor 2) is shown for all 4 time points across all 8 assayed CARs in CD4 and CD8 T cells.

FIG. 16 summarizes the results of additional experiments performed to illustrate that differential costimulatory domains alter proliferation dynamics. These experiments are described in FIG. 13. Here both donors are combined into a relative mean proliferation for each time point for CD4 and CD8 T cells separately. Individual CARs are ranked for each time point, with more proliferative CARs shown on the left-hand side of each subpanel.

FIG. 17 summarizes the results of the CD8 T cell Incucyte cytotoxicity assay. In these experiments, specific cytotoxicity was measured in an live cell time-lapse imaging assay, where mKate-labelled K562 target cells were measured alongside CAR-transduced T cells, which were sorted out of a long-term culture at various times after continuous stimulation with irradiated K562s. Killing is shown as percent of target cells remaining after 32 hours in culture when compared to K562 cells plated with untransduced T cells.

FIG. 18 summarizes the results of experiments of the CD4 T cell Incucyte cytotoxicity assay. In these experiments, specific cytotoxicity was measured in an live cell time-lapse imaging assay, where mKate-labelled K562 target cells are measured alongside CAR-transduced T cells, which are sorted out of a long-term culture at various times after continuous stimulation with irradiated K562s. Killing is shown as percent of target cells remaining after 80 hours in culture when compared to K562 cells plated with untransduced T cells.

FIG. 19 summarizes the results of Incucyte killing assays. Killing was measured in an live cell time-lapse imaging assay, where mKate-labelled K562 target cells are measured alongside CAR-transduced T cells, which are sorted out of a long-term culture at various times after continuous stimulation with irradiated K562s. Killing is shown as tumor cells remaining in culture after 32/80 (CD4/CD8) hours compared to K562 cells plated with untransduced T cells. Percentage (%) killing is averaged between donors.

FIG. 20 graphically summarizes the results of experiments performed to illustrate that CD27 expression is maintained in BAFF-R and TACI. CD27 was stained at various timepoints for each CAR. CD27 expression is maintained in BAFF-R and TACI CARs similar to untransduced T cells, while CD27 expression is lost in 4-1BB and CD28.

FIG. 21 graphically summarizes the results of experiments performed to illustrate that CD28, 4-1BB, and Baffr are highest cytokine producers. In these experiments, primary human CD4 T cells were lentivirally transduced with each CAR, sorted for purity, and stimulated with CD19+or CD19- irradiated K562 tumor cells every three days over a period of 33 days. At 24 hours, 4 days, 10 days, 19 days, and 28 days, the cells were assayed for cytokine production. CD28 was the highest producer of IFNγ and TNFα, 4-1BB was the highest producer of IL-2, and BAFF-R as well as TACI produced similar levels of TNFα as compared to 4-1BB. The number within each box represents the percent of the total T cell population that was producing each cytokine.

FIG. 22 graphically summarizes the results of experiments where arrayed CARs demonstrate differential signaling dynamics. In these experiments, mCherry-reporter Jurkat cell lines for AP1, NFAT, and NFkB were lentivirally transduced with each CAR, sorted for purity and expression, and stimulated with Cd19+or CD19- K562 tumor cells. Transcription factor induction was measured via flow cytometry 8, 24, and 48 hours later. Results indicate an antigen specific induction of all three transcription factors with BAFF-R, TACI, and TNR8 (CD30) showing the highest transcription factor activation.

FIG. 23 depicts a qualitative summary of the arrayed screen, where the CARs were evaluated for the following properties: killing, proliferation, pro-memory, and anti-exhaustion.

FIG. 24 schematically summarizes the results of experiments performed to evaluate CD4 differentiation subsets over time. In these experiments, T cell differentiation subsets are plotted as stacked bar graphs for each day of the arrayed assay. Naive T cells (CD27+/CD45RO−) transition to T Effector cells (TEM) after activation (CD27−/CD45RO+). T central memory cells (TCM) are capable of mounting a long-term immune response upon rechallenge (CD27+/CDRO+). TEMRA (T Effector Memory RA+) cells are less proliferative and have reduced polyfunctionality, and are the final differentiated state of T cells before apoptosis/death. As shown in FIG. 24, TACI, 4-1BB and BAFF-R cells have increased TCM and reduced TEMRA populations at later time points, though the effect is variable between donors.

FIG. 25 schematically summarizes the results of experiments performed to evaluate JCD8 differentiation subsets over time. In these experiments, T cell differentiation subsets are plotted as stacked bar graphs for each day of the arrayed assay. Naive T cells (CD27+/CD45RO−) transition to T Effector cells (TEM) after activation (CD27-/CD45RO+). T central memory cells (TCM) are capable of mounting a long-term immune response upon rechallenge (CD27+/CDRO+). TEMRA (T Effector Memory RA+) cells are less proliferative and have reduced cytokine polyfunctionality, and are the final differentiated state of T cells before apoptosis/death. As shown in FIG. 25, CD40 cells have increased TCM and reduced TEMRA populations at later time points, though the effect is variable between donors.

FIG. 26 schematically summarizes the results from experiments performed to tumor clearance between a CAR against CD19 (which is not normally expressed by M28 tumors and was exogenously expressed), and ALPPL2 (which is a natural tumor antigen on M28). In these experiments, 4×106 M28 mesothelioma tumor cells were subcutaneously injected into flank of NSG mice. Seven days later, 6×10⁶4-1BB CART cells targeting either ALPP2 or CD19 were injected intravenously. Untransduced T cells and non-treated mice were included as controls. Tumors were measured via caliper every 7 days for a total of 30 days.

FIG. 27 schematically summarizes the results from experiments performed to validate the in vitro cytotoxicity assay observation in an in vivo setting by using an established epithelioid mesothelioma solid tumor model (M28). Tumor size was monitored over 49 days post tumor injection. CAR T cell variants are compared to untransduced T cells and no T cell controls.

### DETAILED DESCRIPTION OF THE DISCLOSURE

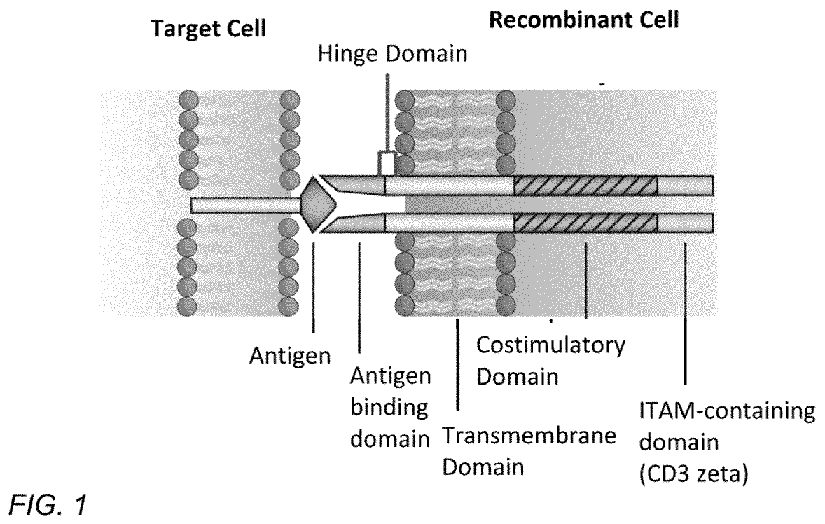

The present disclosure relates generally to chimeric receptors (CRs) and their signaling components for the regulation of an immune response, including CRs with properties that can be tuned to achieve desired functionality by ways of varying their signaling components and the arrangements of these components in the CRs. This is because many existing recombinant chimeric receptors (often referred to as 1^{st} generation chimeric receptors) can only function as an “on/off” switch with no or little mean to allow for fine-tuning their activity and/or functionality. Among the provided embodiments are chimeric receptors (CRs) including one or more intracellular immunomodulatory signaling domains (SDs) (e.g., co-stimulatory domains and/or inhibitory domains). As shown schematically in FIG. 1, the recombinant receptors of the disclosure generally include an extracellular binding domain capable of binding to a target antigen, which is followed by a short hinge domain which can alter expression, dimerization, and binding. The CRs of the disclosure can also contain a TMD, a costimulatory domain (whose modification will be described in greater detail below), and an ITAM-containing C-terminal domain, which is usually the intracellular region of the CD3 zeta chain.

CR-expressing immune cells can be used as programmable effector cells for adoptive cell therapy, engineered to specifically shape an immune response based on the presence or absence of a number of different factors, such as specific cells or circulating molecules. Accordingly, embodiments of the present disclosure are directed to chimeric receptors with a signaling region that is capable of modulating an immune response, such as upon binding a specific antigen, for use in cell therapies, including, but not limited to, antigen-specific cell therapies.

The present disclosure generally relates to, inter alia, new discoveries based on optimizing the intracellular signaling region of a chimeric receptor for a given effector immune cell function, e.g., an effector T cell function. This includes, but is not limited to, incorporating the SDs or combinations of SDs embodied herein, subsets of their sequences, domains derived from other species or viruses, non-immune SDs, synthetic domains or combinations thereof These signaling architectures designed to maximize immune cell effector function can be present in any chimeric receptor, such as those including an extracellular antigen recognition moiety including, but not limited to, a single chain antibody fragment (scFv) or another type of antibody-based molecule, or a functional non-T cell receptor, or any other antigen recognition molecule.
A. Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof “A and/or B” is used herein to include all of the following alternatives: “A”, “B”, “A or B”, and “A and B”.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The terms “administration” and “administering”, as used herein, refer to the delivery of a bioactive composition or formulation by an administration route comprising, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

“Cancer” refers to the presence of cells possessing several characteristics typical of cancer-causing cells, such as uncontrolled proliferation, immortality, metastatic potential, rapid growth and proliferation rate, and certain characteristic morphological features. Cancer cells can aggregate into a mass, such as a tumor, or can exist alone within a subject. A tumor can be a solid tumor, a soft tissue tumor, or a metastatic lesion. As used herein, the term “cancer” also encompasses other types of non-tumor cancers. Non-limiting examples include blood cancers or hematological cancers, such as leukemia. Cancer can include premalignant, as well as malignant cancers.

The term “binding affinity” is herein used as a measure of the strength of a non-covalent interaction between two molecules, e.g., an antibody or portion thereof and an antigen. The term “binding affinity” is used to describe monovalent interactions (intrinsic activity). Binding affinity between two molecules may be quantified by determination of the dissociation constant (K_{D}). In turn, K_{D} can be determined by measurement of the kinetics of complex formation and dissociation using, e.g., the surface plasmon resonance (SPR) method (Biacore). The rate constants corresponding to the association and the dissociation of a monovalent complex are referred to as the association rate constants kₐ (or kₒₙ) and dissociation rate constant k_{d} (or k_{off}), respectively. K_{D} is related to kₐ and k_{d} through the equation K_{D}=k_{d}/kₐ. The value of the dissociation constant can be determined directly by well-known methods, and can be computed even for complex mixtures by methods such as those set forth in Caceci et al. (1984, Byte 9: 340-362). For example, the K_{D} may be established using a double-filter nitrocellulose filter binding assay such as that disclosed by Wong & Lohman (1993, Proc. Natl. Acad. Sci. USA 90: 5428- 5432). Other standard assays to evaluate the binding ability of antibodies or polypeptides of the present disclosure towards target antigens are known in the art, including for example, ELISAs, Western blots, RIAs, and flow cytometry analysis, and other assays exemplified elsewhere herein. The binding kinetics and binding affinity of the antibody also can be assessed by standard assays known in the art, such as Surface Plasmon Resonance (SPR), e.g., by using a Biacore™ system, or KinExA.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

A “library” as used herein refers to a system or set of a plurality of different CARs, a system or set of nucleic acids encoding a plurality of different CARs, or a system or set of engineered cells expressing a plurality of different CARs, wherein the different CARs have different signaling domains. In a pooled library, the plurality of CARs, nucleic acids, or engineered cells are present in as a pool in the same container. In an arrayed library, the plurality of CARs, nucleic acids, or engineered cells are present in separate or individual containers.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, and unless otherwise specified, a “therapeutically effective amount” or a “therapeutically effective number” of an agent is an amount or number sufficient to provide a therapeutic benefit in the treatment or management of a disease, e.g., cancer, or to delay or minimize one or more symptoms associated with the disease. A therapeutically effective amount or number of a compound means an amount or number of therapeutic agent, alone or in combination with other therapeutic agents, which provides a therapeutic benefit in the treatment or management of the disease. The term “therapeutically effective amount” can encompass an amount or number that improves overall therapy of the disease, reduces or avoids symptoms or causes of the disease, or enhances therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human subjects) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, e.g., sheep, dogs, cows, chickens, amphibians, reptiles, etc. In some cases, the methods of the present disclosure find use in experimental animals, in veterinary application, and in the development of animal models for disease, including, but not limited to, rodents including mice, rats, and hamsters, and primates.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In some embodiments, the genes, nucleic acid sequences, amino acid sequences, peptides, polypeptides and proteins are human. The term “gene” is also intended to include variants thereof

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.
B. Compositions of the Disclosure

As described in greater detail below, one aspect of the present disclosure relates to a new class of chimeric receptors (CRs) engineered to include an intracellular signaling domain (SD) whose identity demonstrates an impact on effector function of immune cells expressing the CR. Also provided, in other related aspects of the disclosure, are nucleic acids encoding the CARs as disclosed herein, recombinant immune cells expressing the CARs as disclosed herein, pharmaceutical compositions containing the nucleic acids and/or recombinant cells as disclosed herein.
Chimeric Receptors (CRs)

In one aspect, some embodiments of the disclosure relate to a chimeric receptor (CR) which includes: a) an extracellular domain; b) a transmembrane domain; and c) an intracellular signal transduction domain including an SD derived from a signaling molecule selected from the group consisting of 4-1BB, BAFF-R, BCMA, BTLA, CD2, CD200R, CD244, CD28, CD300a, CD300f, CD40, CD7, CD72, CD96, CRACC, CRTAM, CTLA4, CXADR, DC-SIGN, GITR, HAVCR2, ICOS, ILT2, ILT3, ILT4, KIR2DL1, KIR3DL1, KLRG1, LAG3, LAIR1, NKG2D, NKR-P1A, NTB-A, PD1, Siglec-3, TACI, TIGIT, TLT-1, and TNR8 (CD30).

An SD derived from a signaling molecule includes fragments of the signaling molecule and variants thereof that retain all, substantially all, or at least some (such as at least 20%, 30%, 40%, 50%, 60%, 70%, 80%, 90%, 91%, 92%, 93%, 94%, 95%, 96%, 97%, 98%, 99%, or greater) of the signaling activity of the signaling molecule when included in a CR. In some embodiments, the extracellular domain is an antigen-binding domain capable of binding to one or more target antigens. In some embodiments, binding of a target antigen to a cognate extracellular antigen-binding domain of the CR allows for signaling though the signal transduction domain to occur. CRs can interact with CARs to co-stimulate or inhibit CAR activity, among other functions.

In some embodiments, according to any of the CRs described herein, the SD is derived from a signaling molecule selected from the group consisting of CD2, CD226, CRTAM, B71, ICOS, CXAR, CD7, NTB-A, CD200R, NKR-P1A, OX40, GITR, CD27, CD40, and TNR8 (CD30). In some embodiments, the SD is derived from CD2. In some embodiments, the SD is derived from CD2 and includes the amino acid sequence of SEQ ID NO: 19 or a variant thereof having at least about 80%, about 85%, about 90%, about 95%, about 96%, about 97%, about 98%, about 99%, or about 100% sequence identity to the amino acid sequence of SEQ ID NO: 19. In some embodiments, the SD is derived from CD226. In some embodiments, the SD is derived from CD226 and includes the amino acid sequence of SEQ ID NO: 9 or a variant thereof having at least about 80%, about 85%, about 90%, about 95%, about 96%, about 97%, about 98%, about 99%, or about 100% sequence identity to the amino acid sequence of SEQ ID NO: 9.

In some embodiments, the SD is derived from CRTAM. In some embodiments, the SD is derived from CRTAM and includes the amino acid sequence of SEQ ID NO: 13 or a variant thereof having at least about 80%, about 85%, about 90%, about 95%, about 96%, about 97%, about 98%, about 99%, or about 100% sequence identity to the amino acid sequence of SEQ ID NO: 13. In some embodiments, the SD is derived from B71. In some embodiments, the SD is derived from B7-1 and includes the amino acid sequence of SEQ ID NO: 102 or a variant thereof having at least about 80%, about 85%, about 90%, about 95%, about 96%, about 97%, about 98%, about 99%, or about 100% sequence identity to the amino acid sequence of SEQ ID NO: 102. In some embodiments, the SD is derived from ICOS. In some embodiments, the SD is derived from ICOS and includes the amino acid sequence of SEQ ID NO: 1 or 47 or a variant thereof having at least about 80%, about 85%, about 90%, about 95%, about 96%, about 97%, about 98%, about 99%, or about 100% sequence identity to the amino acid sequence of SEQ ID NO: 1 or 47.

In some embodiments, the SD is derived from CXAR. In some embodiments, the SD is derived from CXAR and includes the amino acid sequence of SEQ ID NO: 17 or a variant thereof having at least about 80%, about 85%, about 90%, about 95%, about 96%, about 97%, about 98%, about 99%, or about 100% sequence identity to the amino acid sequence of SEQ ID NO: 17. In some embodiments, the SD is derived from CD7. In some embodiments, the SD is derived from CD7 and includes the amino acid sequence of SEQ ID NO: 3 or a variant thereof having at least about 80%, about 85%, about 90%, about 95%, about 96%, about 97%, about 98%, about 99%, or about 100% sequence identity to the amino acid sequence of SEQ ID NO: 3. In some embodiments, the SD is derived from NTB-A. In some embodiments, the SD is derived from NTB-A and includes the amino acid sequence of SEQ ID NO: 29 or a variant thereof having at least about 80%, about 85%, about 90%, about 95%, about 96%, about 97%, about 98%, about 99%, or about 100% sequence identity to the amino acid sequence of SEQ ID NO: 29.

In some embodiments, the SD is derived from CD200R. In some embodiments, the SD is derived from CD200R and includes the amino acid sequence of SEQ ID NO: 40 or a variant thereof having at least about 80%, about 85%, about 90%, about 95%, about 96%, about 97%, about 98%, about 99%, or about 100% sequence identity to the amino acid sequence of SEQ ID NO: 40. In some embodiments, the SD is derived from NKR-P1A. In some embodiments, the SD is derived from NKR-P1A and includes the amino acid sequence of SEQ ID NO: 33 or a variant thereof having at least about 80%, about 85%, about 90%, about 95%, about 96%, about 97%, about 98%, about 99%, or about 100% sequence identity to the amino acid sequence of SEQ ID NO: 33. In some embodiments, the SD is derived from OX40. In some embodiments, the SD is derived from OX40 and includes the amino acid sequence of SEQ ID NO: 110 or a variant thereof having at least about 80%, about 85%, about 90%, about 95%, about 96%, about 97%, about 98%, about 99%, or about 100% sequence identity to the amino acid sequence of SEQ ID NO: 110.

In some embodiments, the SD is derived from GITR. In some embodiments, the SD is derived from GITR and includes the amino acid sequence of SEQ ID NO: 8 or a variant thereof having at least about 80%, about 85%, about 90%, about 95%, about 96%, about 97%, about 98%, about 99%, or about 100% sequence identity to the amino acid sequence of SEQ ID NO: 8. In some embodiments, the SD is derived from CD30. In some embodiments, the SD is derived from CD30 and includes the amino acid sequence of SEQ ID NO: 21 or a variant thereof having at least about 80%, about 85%, about 90%, about 95%, about 96%, about 97%, about 98%, about 99%, or about 100% sequence identity to the amino acid sequence of SEQ ID NO: 21. In some embodiments, the SD is derived from CD27. In some embodiments, the SD is derived from CD27 and includes the amino acid sequence of SEQ ID NO: 6 or a variant thereof having at least about 80%, about 85%, about 90%, about 95%, about 96%, about 97%, about 98%, about 99%, or about 100% sequence identity to the amino acid sequence of SEQ ID NO: 6. In some embodiments, the SD is derived from CD40. In some embodiments, the SD is derived from CD40 and includes the amino acid sequence of SEQ ID NO: 10 or a variant thereof having at least about 80%, about 85%, about 90%, about 95%, about 96%, about 97%, about 98%, about 99%, or about 100% sequence identity to the amino acid sequence of SEQ ID NO: 10.

In some embodiments, the SD is derived from TNR8 (CD30). In some embodiments, the SD is derived from TNR8 (CD30) and includes the amino acid sequence of SEQ ID NO: 10 or a variant thereof having at least about 80%, about 85%, about 90%, about 95%, about 96%, about 97%, about 98%, about 99%, or about 100% sequence identity to the amino acid sequence of SEQ ID NO: 10. In some embodiments, the SD is derived from TNFRSF25. In some embodiments, the SD is derived from TNFRSF25 and includes the amino acid sequence of SEQ ID NO: 15 or a variant thereof having at least about 80%, about 85%, about 90%, about 95%, about 96%, about 97%, about 98%, about 99%, or about 100% sequence identity to the amino acid sequence of SEQ ID NO: 15. In some embodiments, the SD is derived from CD244 and includes the amino acid sequence of SEQ ID NO: 118 or a variant thereof having at least about 80%, about 85%, about 90%, about 95%, about 96%, about 97%, about 98%, about 99%, or about 100% sequence identity to the amino acid sequence of SEQ ID NO: 118. In some embodiments, the SD is derived from HAVCR2 and includes the amino acid sequence of SEQ ID NO: 120 or a variant thereof having at least about 80%, about 85%, about 90%, about 95%, about 96%, about 97%, about 98%, about 99%, or about 100% sequence identity to the amino acid sequence of SEQ ID NO: 120. In some embodiments, the CR is a co-stimulatory CR capable of mediating a co-stimulatory signal through its SD.

In some embodiments, according to any of the CRs described herein, the intracellular SD is an inhibitory SD derived from a signaling molecule selected from the group consisting of 2B4, LAG3, CRACC, TIGIT, BTLA, LAIR1, PD-1, CTLA4, ILT2, ILT3, ILT4, KLRG1, KIR3DL1, KIR2DL1, TIM-3, TLT-1, Allergin-1, and PIR-B. In some embodiments, the SD is derived from 2B4. In some embodiments, the SD is derived from 2B4 and includes the amino acid sequence of SEQ ID NO: 20 or a variant thereof having at least about 80% sequence identity to the amino acid sequence of SEQ ID NO: 20. In some embodiments, the SD is derived from LAG3. In some embodiments, the SD is derived from LAG3 and includes the amino acid sequence of SEQ ID NO: 7 or a variant thereof having at least about 80%, about 85%, about 90%, about 95%, about 96%, about 97%, about 98%, about 99%, or about 100% sequence identity to the amino acid sequence of SEQ ID NO: 7. In some embodiments, the SD is derived from CRACC. In some embodiments, the SD is derived from CRACC and includes the amino acid sequence of SEQ ID NO: 30 or a variant thereof having at least about 80% sequence identity to the amino acid sequence of SEQ ID NO: 30.

In some embodiments, the SD is derived from TIGIT. In some embodiments, the SD is derived from TIGIT and includes the amino acid sequence of SEQ ID NO: 12 or a variant thereof having at least about 80%, about 85%, about 90%, about 95%, about 96%, about 97%, about 98%, about 99%, or about 100% sequence identity to the amino acid sequence of SEQ ID NO: 12. In some embodiments, the SD is derived from BTLA. In some embodiments, the SD is derived from BTLA and includes the amino acid sequence of SEQ ID NO: 18 or a variant thereof having at least about 80%, about 85%, about 90%, about 95%, about 96%, about 97%, about 98%, about 99%, or about 100% sequence identity to the amino acid sequence of SEQ ID NO: 18. In some embodiments, the SD is derived from LAIR1. In some embodiments, the SD is derived from LAIR1 and includes the amino acid sequence of SEQ ID NO: 16 or a variant thereof having at least about 80%, about 85%, about 90%, about 95%, about 96%, about 97%, about 98%, about 99%, or about 100% sequence identity to the amino acid sequence of SEQ ID NO: 16.

In some embodiments, the SD is derived from PD-1. In some embodiments, the SD is derived from PD-1 and includes the amino acid sequence of SEQ ID NO: 14 or a variant thereof having at least about 80%, about 85%, about 90%, about 95%, about 96%, about 97%, about 98%, about 99%, or about 100% sequence identity to the amino acid sequence of SEQ ID NO: 14. In some embodiments, the SD is derived from CTLA4. In some embodiments, the SD is derived from CTLA4 and includes the amino acid sequence of SEQ ID NO: 2 or a variant thereof having at least about 80%, about 85%, about 90%, about 95%, about 96%, about 97%, about 98%, about 99%, or about 100% sequence identity to the amino acid sequence of SEQ ID NO: 2. In some embodiments, the SD is derived from ILT2. In some embodiments, the SD is derived from ILT2 and includes the amino acid sequence of SEQ ID NO: 34 or a variant thereof having at least about 80% sequence identity to the amino acid sequence of SEQ ID NO: 34. In some embodiments, the SD is derived from ILT3. In some embodiments, the SD is derived from ILT3 and includes the amino acid sequence of SEQ ID NO: 37 or a variant thereof having at least about 80%, about 85%, about 90%, about 95%, about 96%, about 97%, about 98%, about 99%, or about 100% sequence identity to the amino acid sequence of SEQ ID NO: 37. In some embodiments, the SD is derived from ILT4. In some embodiments, the SD is derived from ILT4 and includes the amino acid sequence of SEQ ID NO: 38 or a variant thereof having at least about 80%, about 85%, about 90%, about 95%, about 96%, about 97%, about 98%, about 99%, or about 100% sequence identity to the amino acid sequence of SEQ ID NO: 38.

In some embodiments, the SD is derived from KLRG1. In some embodiments, the SD is derived from KLRG1 and includes the amino acid sequence of SEQ ID NO: 32 or a variant thereof having at least about 80%, about 85%, about 90%, about 95%, about 96%, about 97%, about 98%, about 99%, or about 100% sequence identity to the amino acid sequence of SEQ ID NO: 32. In some embodiments, the SD is derived from KIR3DL1. In some embodiments, the SD is derived from KIR3DL1 and includes the amino acid sequence of SEQ ID NO: 36 or a variant thereof having at least about 80%, about 85%, about 90%, about 95%, about 96%, about 97%, about 98%, about 99%, or about 100% sequence identity to the amino acid sequence of SEQ ID NO: 36. In some embodiments, the SD is derived from KIR2DL1. In some embodiments, the SD is derived from KIR2DL1 and includes the amino acid sequence of SEQ ID NO: 35 or a variant thereof having at least about 80%, about 85%, about 90%, about 95%, about 96%, about 97%, about 98%, about 99%, or about 100% sequence identity to the amino acid sequence of SEQ ID NO: 35.

In some embodiments, the SD is derived from TIM-3. In some embodiments, the SD is derived from TIM-3 and includes the amino acid sequence of SEQ ID NO: 11 or a variant thereof having at least about 80%, about 85%, about 90%, about 95%, about 96%, about 97%, about 98%, about 99%, or about 100% sequence identity to the amino acid sequence of SEQ ID NO: 11. In some embodiments, the SD is derived from TLT-1. In some embodiments, the SD is derived from TLT-1 and includes the amino acid sequence of SEQ ID NO: 39 or a variant thereof having at least about 80%, about 85%, about 90%, about 95%, about 96%, about 97%, about 98%, about 99%, or about 100% sequence identity to the amino acid sequence of SEQ ID NO: 39. In some embodiments, the SD is derived from Allergin-1. In some embodiments, the SD is derived from Allergin-1 and includes the amino acid sequence of SEQ ID NO: 44 or a variant thereof having at least about 80%, about 85%, about 90%, about 95%, about 96%, about 97%, about 98%, about 99%, or about 100% sequence identity to the amino acid sequence of SEQ ID NO: 44. In some embodiments, the SD is derived from PIR-B. In some embodiments, the SD is derived from PIR-B and includes the amino acid sequence of SEQ ID NO: 45 or a variant thereof having at least about 80%, about 85%, about 90%, about 95%, about 96%, about 97%, about 98%, about 99%, or about 100% sequence identity to the amino acid sequence of SEQ ID NO: 45. In some embodiments, the CR is an inhibitory CR capable of mediating an inhibitory signal through its SD.

In some embodiments, according to any of the CRs described herein, the intracellular SD is stimulatory or inhibitory, and is derived from a signaling molecule selected from the group consisting of LY9, TIM-1, NKG2D, SIGLEC-3, CD72, DC-SIGN, TACI, BAFF-R, and BCMA. In some embodiments, the SD is derived from LY9. In some embodiments, the SD is derived from LY9 and includes the amino acid sequence of SEQ ID NO: 106 or a variant thereof having at least about 80%, about 85%, about 90%, about 95%, about 96%, about 97%, about 98%, about 99%, or about 100% sequence identity to the amino acid sequence of SEQ ID NO: 106. In some embodiments, the SD is derived from TIM-1. In some embodiments, the SD is derived from TIM-1 and includes the amino acid sequence of SEQ ID NO: 23 or a variant thereof having at least about 80%, about 85%, about 90%, about 95%, about 96%, about 97%, about 98%, about 99%, or about 100% sequence identity to the amino acid sequence of SEQ ID NO: 23. In some embodiments, the SD is derived from NKG2D. In some embodiments, the SD is derived from NKG2D and includes the amino acid sequence of SEQ ID NO: 22 or a variant thereof having at least about 80%, about 85%, about 90%, about 95%, about 96%, about 97%, about 98%, about 99%, or about 100% sequence identity to the amino acid sequence of SEQ ID NO: 22.

In some embodiments, the SD is derived from SIGLEC-3. In some embodiments, the SD is derived from SIGLEC-3 and includes the amino acid sequence of SEQ ID NO: 31 or a variant thereof having at least about 80%, about 85%, about 90%, about 95%, about 96%, about 97%, about 98%, about 99%, or about 100% sequence identity to the amino acid sequence of SEQ ID NO: 31. In some embodiments, the SD is derived from CD72. In some embodiments, the SD is derived from CD72 and includes the amino acid sequence of SEQ ID NO: 27 or a variant thereof having at least about 80%, about 85%, about 90%, about 95%, about 96%, about 97%, about 98%, about 99%, or about 100% sequence identity to the amino acid sequence of SEQ ID NO: 27. In some embodiments, the SD is derived from DC-SIGN. In some embodiments, the SD is derived from DC-SIGN and includes the amino acid sequence of SEQ ID NO: 43 or a variant thereof having at least about 80%, about 85%, about 90%, about 95%, about 96%, about 97%, about 98%, about 99%, or about 100% sequence identity to the amino acid sequence of SEQ ID NO: 43.

In some embodiments, the SD is derived from TACI. In some embodiments, the SD is derived from TACI and includes the amino acid sequence of SEQ ID NO: 25 or a variant thereof having at least about 80%, about 85%, about 90%, about 95%, about 96%, about 97%, about 98%, about 99%, or about 100% sequence identity to the amino acid sequence of SEQ ID NO: 25. In some embodiments, the SD is derived from BAFF-R. In some embodiments, the SD is derived from BAFF-R and includes the amino acid sequence of SEQ ID NO: 24 or a variant thereof having at least about 80%, about 85%, about 90%, about 95%, about 96%, about 97%, about 98%, about 99%, or about 100% sequence identity to the amino acid sequence of SEQ ID NO: 24. In some embodiments, the SD is derived from BCMA. In some embodiments, the SD is derived from BCMA and includes the amino acid sequence of SEQ ID NO: 26 or a variant thereof having at least about 80%, about 85%, about 90%, about 95%, about 96%, about 97%, about 98%, about 99%, or about 100% sequence identity to the amino acid sequence of SEQ ID NO: 26. In some embodiments, the CR is a co-stimulatory CR capable of mediating a co-stimulatory signal through its SD. In some embodiments, the CR is an inhibitory CR capable of mediating an inhibitory signal through its SD.

In some embodiments, according to any of the CRs described herein, the intracellular SD is derived from a signaling molecule selected from the group consisting of CD96, CD300f, CD300a, TROY, NGFR, DR6, and RELT. In some embodiments, the SD is derived from CD96. In some embodiments, the SD is derived from CD96 and includes the amino acid sequence of SEQ ID NO: 28 or a variant thereof having at least about 80%, about 85%, about 90%, about 95%, about 96%, about 97%, about 98%, about 99%, or about 100% sequence identity to the amino acid sequence of SEQ ID NO: 28. In some embodiments, the SD is derived from CD300f. In some embodiments, the SD is derived from CD300f and includes the amino acid sequence of SEQ ID NO: 42 or a variant thereof having at least about 80%, about 85%, about 90%, about 95%, about 96%, about 97%, about 98%, about 99%, or about 100% sequence identity to the amino acid sequence of SEQ ID NO: 42. In some embodiments, the SD is derived from CD300a. In some embodiments, the SD is derived from CD300a and includes the amino acid sequence of SEQ ID NO: 41 or a variant thereof having at least about 80%, about 85%, about 90%, about 95%, about 96%, about 97%, about 98%, about 99%, or about 100% sequence identity to the amino acid sequence of SEQ ID NO: 41.

In some embodiments, the SD is derived from TROY. In some embodiments, the SD is derived from TROY and includes the amino acid sequence of SEQ ID NO: 114 or a variant thereof having at least about 80%, about 85%, about 90%, about 95%, about 96%, about 97%, about 98%, about 99%, or about 100% sequence identity to the amino acid sequence of SEQ ID NO: 114. In some embodiments, the SD is derived from NGFR. In some embodiments, the SD is derived from NGFR and includes the amino acid sequence of SEQ ID NO: 108 or a variant thereof having at least about 80%, about 85%, about 90%, about 95%, about 96%, about 97%, about 98%, about 99%, or about 100% sequence identity to the amino acid sequence of SEQ ID NO: 108. In some embodiments, the SD is derived from DR6. In some embodiments, the SD is derived from DR6 and includes the amino acid sequence of SEQ ID NO: 104 or a variant thereof having at least about 80%, about 85%, about 90%, about 95%, about 96%, about 97%, about 98%, about 99%, or about 100% sequence identity to the amino acid sequence of SEQ ID NO: 104. In some embodiments, the SD is derived from RELT. In some embodiments, the SD is derived from RELT and includes the amino acid sequence of SEQ ID NO: 112 or a variant thereof having at least about 80%, about 85%, about 90%, about 95%, about 96%, about 97%, about 98%, about 99%, or about 100% sequence identity to the amino acid sequence of SEQ ID NO: 112.

In some embodiments, according to any of the CRs described herein, the TMD is derived from the same signaling molecule from which the SD is derived. In some embodiments, the TMD and SD in the CR are connected to one another as in the signaling molecule from which they are derived. For example, in some embodiments the CR includes a fragment of a signaling molecule, wherein the fragment of the signaling molecule includes the TMD and an SD of the signaling molecule.

In some embodiments, according to any of the CRs described herein, the TMD is derived from a different signaling molecule than the one from which the SD is derived. For example, in some embodiments the CR includes a TMD from CD28 and an SD from BAFF-R.
Chimeric Antigen Receptors (CAR)

As described in greater detail below, some embodiments of the disclosure relate to novel chimeric antigen receptors (CARs) that contain an extracellular antigen-binding domain and an intracellular signaling domain. In some embodiments, the CAR also includes a TMD. In some embodiments, the CAR's extracellular antigen-binding domain includes (e.g., is composed of) a single chain variable fragment (scFv) derived from a fusion protein of the variable regions of the heavy and light chains of an antibody. In some embodiments, also useful are scFvs derived from Fab fragments (instead of from an antibody, e.g., obtained from Fab libraries). In some embodiments, the CAR of the disclosure includes a scFv fused to the TMD and then to the intracellular signaling domain. “First-generation” CARs include those that solely provide CD3-chain induced signal upon antigen binding. “Second-generation” CARs include those that provide both CD3-chain induced signal upon antigen binding and co-stimulation, such as one including an intracellular signaling domain from a costimulatory receptor (e.g., CD28 or 4-1BB). “Third-generation” CARs include those that include multiple co-stimulatory domains of different costimulatory receptors. A fourth generation of CAR-T cell includes CAR-T cells redirected for cytokine killing (TRUCK), where the vector containing the CAR construct includes a cytokine cassette. Once the CAR-T cell is activated, it can deposits a pro-inflammatory or cytotoxic cytokine into the tumor lesion.

In some embodiments, according to any of the CRs described herein including a co-stimulatory SD, the intracellular signal transduction domain further includes an activation domain. In some embodiments, the activation domain includes one or more immunoreceptor tyrosine-based activation motifs (ITAMs). In some embodiments, the activation domain is derived from CD3. In some embodiments, the extracellular domain is an antigen-binding domain capable of binding to a target antigen. In some embodiments, the extracellular antigen-binding domain includes an antibody moiety capable of binding to the target antigen. In some embodiments, the antibody moiety is a scFv. In some embodiments, the CR is a chimeric antigen receptor (CAR).

In some embodiments, provided herein is a CAR including (a) an extracellular antigen-binding domain capable of binding to a target antigen; (b) a transmembrane domain (TMD); and (c) an intracellular signal transduction domain including an intracellular SD derived from a signaling molecule selected from the group consisting of 4-1BB, BAFF-R, BCMA, BTLA, CD2, CD200R, CD244, CD28, CD300a, CD300f, CD40, CD7, CD72, CD96, CRACC, CRTAM, CTLA4, CXADR, DC-SIGN, GITR, HAVCR2, ICOS, ILT2, ILT3, ILT4, KIR2DL1, KIR3DL1, KLRG1, LAG3, LAIR1, NKG2D, NKR-P1A, NTB-A, PD1, Siglec-3, TACI, TIGIT, TLT-1, and TNR8 (CD30), and derivatives, mutants, variants, fragments and combinations thereof

In some embodiments, the extracellular antigen-binding domain includes an antibody, an antibody derivative, a T cell receptor variable region, a soluble T cell receptor, an aptamer, a nanobody, an extracellular domain of a receptor, a receptor ligand, or a fragment or combination thereof. In some embodiments, the co-stimulatory SD is derived from a signaling molecule selected from the group consisting BAFF-R, CD40, TACI, CD2, CD7, CD30, and NTB-A. In some embodiments, the chimeric receptors of the disclosure further include a hinge domain inserted between the extracellular antigen-binding domain and the TMD. In some embodiments, the hinge domain includes a flexible polypeptide connector region providing structural flexibility and spacing to flanking polypeptide regions. The hinge can consist of natural or synthetic polypeptides.

In some embodiments, the primary SD is or includes the CD3 chain domain, wherein the CD3 chain is selected from the group consisting of: a CD3 zeta (CD3) chain, a CD3 gamma (CD3y) chain, a CD3 delta (CD3δ) chain, a CD3 epsilon (CD3c) chain, derivatives, mutants, variants, fragments and combinations thereof. In certain embodies, the primary SD optionally further includes an Fc domain from the immunoglobulin superfamily, such as for example, FcγRI (CD64), FcγRIIA (CD32), FcγRIIB (CD32), FcγRIIIA (CD16a), FcγRIIIB (CD16b), FcαRI (CD89), FcϵRI, FcϵRII (CD23), Fcα, FcμR, derivatives, mutants, variants, fragments and combinations thereof. In some embodiments, the Fc domain is an Fcγ domain, derivatives, mutants, variants, fragments and combinations thereof. As used herein, an “Fcγdomain” includes FcγRI (CD64), FcγRIIA (CD32), FcγRIIB (CD32), FcγRIIIA (CD16a), FcγRIIIB (CD16b) derivatives, mutants, variants, and fragments thereof

In some embodiments, a co-stimulatory SD is derived from a signaling molecule selected from the group consisting of CD2, CD226, CRTAM, B71, ICOS, CXAR, CD7, NTB-A, CD200R, NKR-P1A, OX40, GITR, CD27, CD40, and TNR8 (CD30). In some embodiments, the SD is derived from CD2. In some embodiments, the SD is derived from CD2 and includes the amino acid sequence of SEQ ID NO: 19 or a variant thereof having at least about 80%, about 85%, about 90%, about 95%, about 96%, about 97%, about 98%, about 99%, or about 100% sequence identity to the amino acid sequence of SEQ ID NO: 19. In some embodiments, the SD is derived from CD226. In some embodiments, the SD is derived from CD226 and includes the amino acid sequence of SEQ ID NO: 9 or a variant thereof having at least about 80%, about 85%, about 90%, about 95%, about 96%, about 97%, about 98%, about 99%, or about 100% sequence identity to the amino acid sequence of SEQ ID NO: 9. In some embodiments, the SD is derived from CRTAM. In some embodiments, the SD is derived from CRTAM and includes the amino acid sequence of SEQ ID NO: 13 or a variant thereof having at least about 80%, about 85%, about 90%, about 95%, about 96%, about 97%, about 98%, about 99%, or about 100% sequence identity to the amino acid sequence of SEQ ID NO: 13. In some embodiments, the SD is derived from B71. In some embodiments, the SD is derived from B7-1 and includes the amino acid sequence of SEQ ID NO: 102 or a variant thereof having at least about 80%, about 85%, about 90%, about 95%, about 96%, about 97%, about 98%, about 99%, or about 100% sequence identity to the amino acid sequence of SEQ ID NO: 102. In some embodiments, the SD is derived from ICOS. In some embodiments, the SD is derived from ICOS and includes the amino acid sequence of SEQ ID NO: 1 or 47 or a variant thereof having at least about 80%, about 85%, about 90%, about 95%, about 96%, about 97%, about 98%, about 99%, or about 100% sequence identity to the amino acid sequence of SEQ ID NO: 1 or 47.

In some embodiments, the SD is derived from CXAR. In some embodiments, the SD is derived from CXAR and includes the amino acid sequence of SEQ ID NO: 17 or a variant thereof having at least about 80%, about 85%, about 90%, about 95%, about 96%, about 97%, about 98%, about 99%, or about 100% sequence identity to the amino acid sequence of SEQ ID NO: 17. In some embodiments, the SD is derived from CD7. In some embodiments, the SD is derived from CD7 and includes the amino acid sequence of SEQ ID NO: 3 or a variant thereof having at least about 80%, about 85%, about 90%, about 95%, about 96%, about 97%, about 98%, about 99%, or about 100% sequence identity to the amino acid sequence of SEQ ID NO: 3. In some embodiments, the SD is derived from NTB-A. In some embodiments, the SD is derived from NTB-A and includes the amino acid sequence of SEQ ID NO: 29 or a variant thereof having at least about 80%, about 85%, about 90%, about 95%, about 96%, about 97%, about 98%, about 99%, or about 100% sequence identity to the amino acid sequence of SEQ ID NO: 29. In some embodiments, the SD is derived from CD200R. In some embodiments, the SD is derived from CD200R and includes the amino acid sequence of SEQ ID NO: 40 or a variant thereof having at least about 80%, about 85%, about 90%, about 95%, about 96%, about 97%, about 98%, about 99%, or about 100% sequence identity to the amino acid sequence of SEQ ID NO: 40.

In some embodiments, the SD is derived from NKR-P1A. In some embodiments, the SD is derived from NKR-P1A and includes the amino acid sequence of SEQ ID NO: 33 or a variant thereof having at least about 80%, about 85%, about 90%, about 95%, about 96%, about 97%, about 98%, about 99%, or about 100% sequence identity to the amino acid sequence of SEQ ID NO: 33. In some embodiments, the SD is derived from OX40. In some embodiments, the SD is derived from OX40 and includes the amino acid sequence of SEQ ID NO: 110 or a variant thereof having at least about 80%, about 85%, about 90%, about 95%, about 96%, about 97%, about 98%, about 99%, or about 100% sequence identity to the amino acid sequence of SEQ ID NO: 110. In some embodiments, the SD is derived from GITR. In some embodiments, the SD is derived from GITR and includes the amino acid sequence of SEQ ID NO: 8 or a variant thereof having at least about 80%, about 85%, about 90%, about 95%, about 96%, about 97%, about 98%, about 99%, or about 100% sequence identity to the amino acid sequence of SEQ ID NO: 8. In some embodiments, the SD is derived from CD30. In some embodiments, the SD is derived from CD30 and includes the amino acid sequence of SEQ ID NO: 21 or a variant thereof having at least about 80%, about 85%, about 90%, about 95%, about 96%, about 97%, about 98%, about 99%, or about 100% sequence identity to the amino acid sequence of SEQ ID NO: 21. In some embodiments, the SD is derived from CD27. In some embodiments, the SD is derived from CD27 and includes the amino acid sequence of SEQ ID NO: 6 or a variant thereof having at least about 80%, about 85%, about 90%, about 95%, about 96%, about 97%, about 98%, about 99%, or about 100% sequence identity to the amino acid sequence of SEQ ID NO: 6.

In some embodiments, the SD is derived from CD40. In some embodiments, the SD is derived from CD40 and includes the amino acid sequence of SEQ ID NO: 10 or a variant thereof having at least about 80%, about 85%, about 90%, about 95%, about 96%, about 97%, about 98%, about 99%, or about 100% sequence identity to the amino acid sequence of SEQ ID NO: 10. In some embodiments, the SD is derived from CD30. In some embodiments, the SD is derived from CD30 and includes the amino acid sequence of SEQ ID NO: 10 or a variant thereof having at least about 80%, about 85%, about 90%, about 95%, about 96%, about 97%, about 98%, about 99%, or about 100% sequence identity to the amino acid sequence of SEQ ID NO: 10. In some embodiments, the SD is derived from TNFRSF25. In some embodiments, the SD is derived from TNFRSF25 and includes the amino acid sequence of SEQ ID NO: 15 or a variant thereof having at least about 80%, about 85%, about 90%, about 95%, about 96%, about 97%, about 98%, about 99%, or about 100% sequence identity to the amino acid sequence of SEQ ID NO: 15.

In some embodiments, the CAR may also include a spacer domain situated between the antigen-binding region and T cell plasma membrane. The spacer domain may include a sequence derived from IgG subclass IgG1, IgG4, IgD or CD8. In some embodiments, the spacer domain includes a CD28 motif. The spacer domain can have any length. In some embodiments, the spacer domain includes 1 amino acid or 10 amino acids or 20 amino acids or 50 amino acids or 60 amino acids or 70 amino acids or 80 amino acids or 100 amino acids or 120 amino acids or 140 amino acids or 160 amino acids or 180 amino acids or 200 amino acids or 250 amino acids or 300 amino acids or any number therebetween.

In some embodiments, a CAR may further include a linker region. The linker may be rich in glycine, serine, and/or threonine for solubility/hydrophilicity. The linker region can connect to N-terminus of variable heavy (VH) chain with the C-terminus of the variable light (VL) chain or vice versa.

### Antigen-Binding Domain

In some embodiments, the antigen-binding domain includes one or more antigen-binding determinants of an antibody or a functional antigen-binding fragment thereof. One skilled in the art upon reading the present disclosure will readily understand that the term “functional fragment thereof” or “functional variant thereof” refers to a molecule having biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. For example, an antibody capable of binding to an epitope of a cell surface receptor may be truncated at the N-terminus and/or C-terminus, and the retention of its epitope binding activity assessed using assays known to those of skill in the art.

Numerous antigen-binding domains are known in the art, including those based on the antigen-binding site of an antibody, antibody mimetics, nanobodies, and T-cell receptor fragments. For example, the antigen-binding domain may include: a single-chain variable fragment (scFv) derived from a monoclonal antibody; a natural ligand of the target antigen; a peptide with sufficient binding affinity for the target; a single domain binder such as a camelid; an artificial binder such as a DARPin; or a single-chain derived from a T-cell receptor. Accordingly, the antigen specific binding domain includes, without limitation, an antibody, a T cell receptor fragment, a soluble T cell receptor, nanobody, aptamer, receptors, fragments or combinations thereof. In some embodiments, the antigen specific binding domain is a T cell variable region fragments. In other embodiments, the antigen specific binding domain is an antibody or fragment thereof The CAR can include single chains of T cell receptors and antibodies. In some embodiments, In some embodiments, the antigen-binding domain is selected from the group consisting of an antibody, a nanobody, a diabody, a triabody, or a minibody, a F(ab2)₂ fragment, a Fab fragment, a single chain variable fragment (scFv), and a single domain antibody (sdAb), or a functional fragment thereof. In some embodiments, the antigen-binding domain includes a scFv.

In some embodiments, the antigen-binding domain is or includes an antibody or antibody fragment. In some embodiments, the antibodies are human antibodies, including any known to bind a targeting molecule. The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

The antigen-binding domain can include naturally-occurring amino acid sequences or can be engineered, designed, or modified so as to provide desired and/or improved properties, e.g., binding affinity. Generally, the binding affinity of an antigen-binding domain, e.g., an antibody, for a target antigen (e.g., CD19 antigen) can be calculated by the Scatchard method described by Frankel et al., Mol Immunol (1979) 16:101-06. In some embodiments, binding affinity is measured by an antigen/antibody dissociation rate. In some embodiments, binding affinity is measured by a competition radioimmunoassay. In some embodiments, binding affinity is measured by ELISA. In some embodiments, antibody affinity is measured by flow cytometry. An antibody that “selectively binds” an antigen (such as CD19) is an antigen-binding domain that binds the antigen with high affinity and does not significantly bind other unrelated antigens.

In some embodiments, the antigen-binding domain is a humanized antibody or fragments thereof. A “humanized” antibody is an antibody in which all or substantially all CDR amino acid residues are derived from non-human CDRs and all or substantially all framework region (FR) amino acid residues are derived from human FRs. A humanized antibody optionally may include at least a portion of an antibody constant region derived from a human antibody. A “humanized form” of a non-human antibody, refers to a variant of the non-human antibody that has undergone humanization, in some cases to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In some embodiments, the heavy and light chains of an antibody can be full-length or can be an antigen-binding portion (a Fab, F(ab′)₂, Fv or a single chain Fv fragment (scFv)). In other embodiments, the antibody heavy chain constant region is chosen from, e.g., IgG1, IgG2, IgG3, IgG4, IgM, IgAl, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that includes a portion of an intact antibody that binds the antigen to which the intact antibody binds. Examples of antibody fragments include, but are not limited to, Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In some embodiments, the antibodies are single-chain antibody fragments including a variable heavy chain region and/or a variable light chain region, such as scFvs.

The term “variable region” or “variable domain”, when used in reference to an antibody, such as an antibody fragment, refers to the domain of an antibody heavy or light chain that is involved in binding the antibody to antigen. The variable domains of the heavy chain and light chain (V_{H} and V_{L}, respectively) of a native antibody generally have similar structures, with each domain including four conserved framework regions (FRs) and three CDRs. (See, e.g., Kindt et al. “Kuby Immunology”, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J Immunol (1993) 150:880-87; Clarkson et al., Nature (1991) 352:624-28.

Single-domain antibodies are antibody fragments including all or a portion of the heavy chain variable domain or all or a portion of the light chain variable domain of an antibody. In some embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments including arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.
Regulatory T Cells

In general, T regulatory cells have been identified as a CD4⁺CD25⁺ T cell population capable of suppressing an immune response. The identification of FoxP3 as a “master-regulator” of Tregs helped define Tregs as a distinct T cell lineage. The identification of additional antigenic markers on the surface of Tregs has enabled identification and FACS sorting of viable Tregs to greater purity, resulting in a more highly-enriched and suppressive Treg population. In addition to CD4 and CD25, both mouse and human Tregs express GITR/AITR, CTLA-4, and express low levels of CD127 (IL-7Ra). Moreover, Tregs can exist in different states which can be identified based on their expression of surface markers. Tregs which develop in the thymus from CD4⁺ thymocytes are known as “natural” Tregs, however Tregs can also be induced in the periphery from naive CD4⁺ T cells in response to low-dose engagement of the TCR, TGF beta and IL-2. These “induced” Tregs secrete the immunosuppressive cytokine IL-10. The phenotype of Tregs changes again as they become activated, and markers including GARP in mouse and human, CD45RA in human, and CD103 in mouse have been shown to be useful for the identification of activated Tregs.

Accordingly, in some embodiments, an isolated T cell is modified to express a chimeric antigen receptor (CAR). In some embodiments, the CAR includes an antigen specific binding domain, a spacer domain, a TMD, and an intracellular signaling region, the signaling region including a primary SD, optionally derived from a CD3 chain domain, and a second SD which is a costimulatory or inhibitory SD. In some embodiments, the second SD is a costimulatory or inhibitory SD of a protein selected from the group consisting of: 2B4 , 4-1BB (CD137), Allergin-1, B7-1 (CD80), B7-2 (CD86), BAFF-R , BCMA , BTLA , CD2, CD200R , CD244, CD226, CD27, CD28, CD300a , CD300f , CD40, CD7, CD72, CD96, CRACC , CRTAM , CTLA4, CXADR , DC-SIGN , DR6 (TNFRSF21), GITR , HAVCR2, ICOS , ILT2, ILT3, ILT4, KIR2DL1, KIR3DL1, KLRG1, LAG3, LAIR1, LY9, NGFR, NKG2D , NKR-P1A , NTB-A , OX40 (CD134, TNFRSR4), PD-1, PIR-B , RELT (TNFRSF19L), SIGLEC-3.9.9, TACI , TIGIT , TIM-1, TIM-3, TLT-1, TNR8 (CD30), TNFRSF25, TROY (TNFRSF19), and derivatives, mutants, variants, fragments and combinations thereof.

In some embodiments, the primary SD is or includes a CD3 chain domain. In some embodiments, the CD3 chain is selected from the group consisting of: a CD3 zeta (CD3ζ) chain, a CD3 gamma (CD3γ) chain, a CD3 delta (CD3δ) chain, a CD3 epsilon (CD3ϵ) chain, derivatives, mutants, variants, fragments and combinations thereof.
Inhibitory Receptors (IR)

In some embodiments, according to any of the CRs described, the intracellular signal transduction domain includes a modulatory SD capable of mediating an inhibitory signal derived from a signaling molecule. In some embodiments, the signaling molecule is selected from the group consisting of LY9, TIM-1, NKG2D, SIGLEC-3, CD72, DC-SIGN, TACI, BAFF-R, BCMA, 2B4, LAG3, CRACC, TIGIT, BTLA, LAIR1, PD-1, CTLA4, ILT2, ILT3, ILT4, KLRG1, KIR3DL1, KIR2DL1, TIM-3, TLT-1, Allergin-1, and PIR-B, and derivatives, mutants, variants, fragments and combinations thereof. In some embodiments, the inhibitory SD is derived from a signaling molecule selected from the group consisting KLRG1, DC-SIGN, NKG2D, NKR-P1A. In some embodiments, the inhibitory SD is derived from KLRG1.

In some embodiments, the modulatory SD is derived from KLRG1 and includes the amino acid sequence of SEQ ID NO: 32 or a variant thereof having at least about 80%, about 85%, about 90%, about 95%, about 96%, about 97%, about 98%, about 99%, or about 100% sequence identity to SEQ ID NO: 32. In some embodiments, the modulatory SD is derived from DC-SIGN and includes the amino acid sequence of SEQ ID NO: 43 or a variant thereof having at least about 80%, about 85%, about 90%, about 95%, about 96%, about 97%, about 98%, about 99%, or about 100% sequence identity to SEQ ID NO: 43. In some embodiments, the modulatory SD is derived from NKG2D and includes the amino acid sequence of SEQ ID NO: 22 or a variant thereof having at least about 80%, about 85%, about 90%, about 95%, about 96%, about 97%, about 98%, about 99%, or about 100% sequence identity to SEQ ID NO: 22. In some embodiments, the modulatory SD is derived from NKR-P1A and includes the amino acid sequence of SEQ ID NO: 33 or a variant thereof having at least about 80%, about 85%, about 90%, about 95%, about 96%, about 97%, about 98%, about 99%, or about 100% sequence identity to SEQ ID NO: 33.

Inhibitory CRs can be used to increase the ability of CAR-T cells to discriminate between pathogenic cells and healthy cells, by inhibiting CAR activity in the presence of an antigen that is not characteristic of the target cell. For example, a CAR-T cell may be provided with a CAR specific for HER2 (found in breast tissue and gastric tissue, among others) and an inhibitory CR (IR) specific for CA9 (found in gastric tissue, but not breast). Such a CAR-T cell would be useful for treating breast cancer, with diminished side effects on the stomach (on target off-tumor effects).

In some embodiments, an engineered cell is provided having a CAR that is specific for a target antigen, and an IR that is specific for a non-target antigen, wherein the target antigen is expressed on both target cells and healthy cells, and the non-target antigen is expressed only on healthy cells. In some embodiments, an engineered cell is provided having a CAR that is specific for a target antigen, and an IR that is specific for a non-target antigen, wherein the target antigen is expressed on both target cells and susceptible healthy cells, and the non-target antigen is expressed on susceptible healthy cells that need protection from CAR-T activity.
Switch Receptors (SR)

In some embodiments, according to any of the CRs described herein, the extracellular antigen-binding domain includes an extracellular domain derived from an inhibitory immune checkpoint molecule or a binding moiety capable of binding to a ligand of the inhibitory immune checkpoint molecule. In some embodiments, the CR is a switch receptor (SR) capable of transforming an immune inhibitory signal into an immune stimulatory signal. In some embodiments, the co-stimulatory SD is derived from a signaling molecule selected from the group consisting of CD2, CD226, CRTAM, B71, ICOS, CXAR, CD7, NTB-A, CD200R, NKR-P1A, OX40, GITR, CD30, CD27, CD40, TNFRSF25, LY9, TIM-1, NKG2D, SIGLEC-3, CD72, DC-SIGN, TACI, BAFF-R, and BCMA, and derivatives, mutants, variants, fragments and combinations thereof. In some embodiments, the co-stimulatory SD is derived from a signaling molecule selected from the group consisting BAFF-R, CD40, TACI, CD2, CD7, CD30, and NTB-A.

In some embodiments, according to any of the CRs described, the extracellular antigen-binding domain includes an extracellular domain derived from a stimulatory immune checkpoint molecule or a binding moiety capable of binding to a ligand of the stimulatory immune checkpoint molecule. In some embodiments, the CR is a switch receptor (SR) capable of transforming an immune stimulatory signal into an immune inhibitory signal. In some embodiments, the inhibitory SD is derived from a signaling molecule selected from the group consisting of LY9, TIM-1, NKG2D, SIGLEC-3, CD72, DC-SIGN, TACI, BAFF-R, BCMA, 2B4, LAG3, CRACC, TIGIT, BTLA, LAIR1, PD-1, CTLA4, ILT2, ILT3, ILT4, KLRG1, KIR3DL1, KIR2DL1, TIM-3, TLT-1, Allergin-1, and PIR-B, and derivatives, mutants, variants, fragments and combinations thereof. In some embodiments, the inhibitory SD is derived from a signaling molecule selected from the group consisting KLRG1, DC-SIGN, NKG2D, and NKR-P1A.
Nucleic Acids

In another aspect, some embodiments of the disclosure relate to nucleic acid molecules including nucleotide sequences encoding the chimeric receptors of the disclosure, including expression cassettes, and expression vectors containing these nucleic acid molecules operably linked to heterologous nucleic acid sequences such as, for example, regulatory sequences which allow in vivo expression of the receptor in a host cell or ex-vivo cell-free expression system.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR §1.822 is used herein.

Nucleic acid molecules of the present disclosure can be of any length, including for example, between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, provided herein is a nucleic acid molecule including a nucleotide sequence encoding a chimeric receptor (CR) including, from N-terminus to C-terminus: an extracellular antigen-binding domain capable of binding to a target antigen; b) a transmembrane domain; and c) an intracellular signal transduction domain including an intracellular SD derived from a signaling molecule selected from the group consisting of 4-1BB, BAFF-R, BCMA, BTLA, CD2, CD200R, CD244, CD28, CD300a, CD300f, CD40, CD7, CD72, CD96, CRACC, CRTAM, CTLA4, CXADR, DC-SIGN, GITR, HAVCR2, ICOS, ILT2, ILT3, ILT4, KIR2DL1, KIR3DL1, KLRG1, LAG3, LAIR1, NKG2D, NKR-P1A, NTB-A, PD1, Siglec-3, TACI, TIGIT, TLT-1, and TNR8 (CD30). In some embodiments, the receptor further includes an activating signaling domain. In some embodiments, the activating signaling domain is a CD3 activating SD. In some embodiments, the CD3 activating SD is the SD from the CD3 chain, the CD3γ chain, the CD3δ chain, or the CD3ϵ chain. In some embodiments, the activating SD is the CD3 chain SD.

In some embodiments, the nucleic acid encodes a CR having multiple SDs. In some embodiments, the CR has multiple SDs in addition to an activating SD. In some embodiments, the CR has 1, 2, 3, or 4 SDs. In some embodiments, the CR has 1, 2, 3, or 4 SDs and an activating SD. In some embodiments, the activating SD is the CD3 chain SD.

Nucleic acids of the disclosure can further include one or more polynucleotides of interest. The polynucleotide of interest can be a regulatory or signaling nucleic acid, or can encode any protein that can be expressed by the engineered cell. In some embodiments, the protein is a detectable label. In some embodiments, the detectable label is a fluorescent protein or a chromogenic protein. Suitable fluorescent proteins include, without limitation, GFP, mCherry, mTomato, mStrawberry, and others. In some embodiments, the protein is a therapeutic protein. In some embodiments, the therapeutic protein is a chimeric antigen receptor (CAR). In some embodiments, the therapeutic protein is a therapeutic antibody. In some embodiments, the therapeutic antibody is an antibody capable of specifically binding to an immune checkpoint receptor, such as CTLA-4, PD-1, PD-L1, or others. In some embodiments, the protein is a cytokine. In some embodiments, the cytokine is IL-12 or IFNγ. In some embodiments, the polynucleotide of interest encodes a regulatory nucleic acid. In some embodiments, the regulatory nucleic acid is an RNA. In some embodiments, the regulatory RNA is a siRNA, shRNA, or miRNA.

In some embodiments, the therapeutic protein is an interleukin, a cytokine, or a chemokine. In some embodiments, the regulatory RNA is a siRNA, shRNA, or miRNA. Interleukins are a family of cytokines that (in general) stimulate or regulate immune cells and/or immune functions. For example, IL-1α, IL-1β, IL-18, IL-33, IL-36α, IL-36β, and IL-36γ are all IL-1 family interleukins with proinflammatory properties, and in general induce responses that stimulate immune cells and provide for increased adhesion to endothelial cells in the vicinity of the response. In some embodiments, the therapeutic protein is IL-1α, IL-1β, TNFα, or IL-12.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for the chimeric receptor as disclosed herein, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

The nucleic acid sequences encoding the chimeric receptors can be optimized for expression in the host cell of interest. For example, the G-C content of the sequence can be adjusted to average levels for a given cellular host, as calculated by reference to known genes expressed in the host cell. Methods for codon usage optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the chimeric receptors disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into an individual. An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, e.g., operably linked.

### Engineered Cells and Cell Cultures

The nucleic acids of the disclosure can be introduced into a host cell, such as, for example, a human T lymphocyte, to produce a recombinant engineered cell containing the nucleic acid molecule. Accordingly, some embodiments of the disclosure relate to methods for making recombinant cells, including the steps of: (a) providing a cell capable of protein expression and (b) contacting the provided cell with at least one of the recombinant nucleic acids described herein.

Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, direct micro-injection, nanoparticle-mediated nucleic acid delivery, and the like.

In some embodiments, the nucleic acid molecules are delivered by viral or non-viral delivery vehicles known in the art. For example, the nucleic acid molecule can be stably integrated in the host genome, or can be episomally replicating, or present in the recombinant host cell as a mini-circle expression vector for transient expression. In some embodiments, the nucleic acid molecule is maintained and replicated in the recombinant host cell as an episomal unit. In some embodiments, the nucleic acid molecule is stably integrated into the genome of the recombinant cell. Stable integration can be achieved using classical random genomic recombination techniques, or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant host cell as a mini-circle expression vector for transient expression.

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell (T_{H}), a cytotoxic T cell (T_{CTL}), or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+ T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+ T helper lymphocyte cell selected from the group consisting of naïve CD4+ T cells, central memory CD4+ T cells, effector memory CD4+ T cells, and bulk CD4+ T cells. In some embodiments, the cell are obtained by leukapheresis performed on a sample obtained from a subject. In some embodiments, the subject is a human patient.

As discussed above, some embodiments of the disclosure relate to methods for making a recombinant cell, including (a) providing a cell capable of protein expression and (b) contacting the provided cell with a recombinant nucleic acid of the disclosure. In some embodiments, the method includes providing a plurality of cells capable of protein expression and (b) contacting the plurality of cells with a plurality of recombinant nucleic acids of the disclosure, wherein each nucleic acid encodes a CAR, and the plurality of recombinant nucleic acids encodes a plurality of CARs having different SDs, thus forming a plurality of engineered cells having different CARs. In some embodiments, the plurality includes 2, 3, 4, 5, 6, 7, 8, 9, 10, 12, 15, 20, 25, 30, 35, 40, 41, 42, 43, 44, 45, 46, 47, 48, 49, or about 50 different SDs. In some embodiments, the plurality of cells is contacted with a mixture including a plurality of recombinant nucleic acids. In some embodiments, the plurality of cells is contacted with a plurality of individual recombinant nucleic acids.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.
Pharmaceutical Compositions

In some embodiments, the chimeric receptors, nucleic acids, and recombinant cells of the disclosure can be incorporated into compositions, including pharmaceutical compositions. Such compositions generally include the nucleic acids, and/or recombinant cells, and a pharmaceutically acceptable excipient, e.g., a carrier.

Accordingly, one aspect of the present disclosure relates to pharmaceutical compositions that include a pharmaceutically acceptable carrier and one or more of the following: (a) a chimeric receptor of the disclosure; (b) a recombinant nucleic acid of the disclosure; and/or (c) a recombinant cell of the disclosure. In some embodiments, the composition includes (a) a recombinant nucleic acid of the disclosure and (b) a pharmaceutically acceptable carrier. In some embodiments, the composition is formulated for introducing the recombinant nucleic acid into a cell. In some embodiments, the recombinant nucleic acid is encapsulated in a lipid nanoparticle (LNP), liposome, or viral particle. In some embodiments, the composition includes (a) a recombinant cell of the disclosure and (b) a pharmaceutically acceptable carrier.

In certain embodiments, the pharmaceutical compositions in accordance with some embodiments disclosed herein include cell cultures that can be washed, treated, combined, supplemented, or otherwise altered prior to administration to an individual in need thereof. Furthermore, administration can be at varied doses, time intervals or in multiple administrations.

The pharmaceutical compositions provided herein can be in any form that allows for the composition to be administered to an individual. In some specific embodiments, the pharmaceutical compositions are suitable for human administration. As used herein, the term “pharmaceutically acceptable” means approved by a regulatory agency of the Federal or a state government or listed in the U.S. Pharmacopeia or other generally recognized pharmacopeia for use in animals, and more particularly in humans. The carrier can be a diluent, adjuvant, excipient, or vehicle with which the pharmaceutical composition is administered. Saline solutions and aqueous dextrose and glycerol solutions can also be employed as liquid carriers, including injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. In some embodiments, the pharmaceutical composition is sterilely formulated for administration into an individual. In some embodiments, the individual is a human. One of ordinary skilled in the art will appreciate that the formulation should suit the mode of administration.

In some embodiments, the pharmaceutical compositions of the present disclosure are formulated to be suitable for the intended route of administration to an individual. For example, the pharmaceutical composition may be formulated to be suitable for parenteral, intraperitoneal, colorectal, intraperitoneal, and intratumoral administration. In some embodiments, the pharmaceutical composition may be formulated for intravenous, oral, intraperitoneal, intratracheal, subcutaneous, intramuscular, topical, or intratumoral administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, exemplary methods of preparation include vacuum drying and freeze-drying which yields a powder of the active ingredient plus any additional desired ingredient from a previously sterile-filtered solution thereof.
C. Libraries of Engineered Cells

Engineered cells of the disclosure are further useful for studying the properties of different receptors and modulatory SDs under different conditions. As demonstrated herein, the effect of each SD can vary when using T cells obtained from different donors, thus the SD that is best for one donor may be different from the SD that is best for a different donor. Described herein are methods for making collections of engineered CAR-T cells (a “library”) having a plurality of different SDs, and methods for screening such libraries for activities and functions under a plurality of different experimental circumstances. In some embodiments, the library includes a mixture of engineered cells, having a plurality of CARs having different SDs. In some embodiments, the library includes a collection of engineered cells, having a plurality of CARs having different SDs. In some embodiments, the plurality includes 2, 3, 4, 5, 6, 7, 8, 9, 10, 12, 15, 20, 25, 30, 35, 40, 41, 42, 43, 44, 45, 46, 47, 48, 49, or about 50 different SDs.

There are many parameters of CAR-T cell function and performance, for example, the cell's ability to proliferate in response to its target antigen, its ability to resist exhaustion and survival, its ability to survive despite constant or repeated stimulation, its ability to kill target cells, its ability to secrete cytokines, its ability to ignore immunosuppressive target cell tactics, its ability to differentiate into memory cells, its ability to avoid damaging non-target cells, its ability to survive in a tumor microenvironment, and others. There are also many factors that can affect the ability of a CAR-T cell to perform its functions, such as the genetics and state of the T cells from which the CAR-T cell is generated (e.g., individual genetic variation from donor to donor); the particular characteristics of the target cells, such as the expression level of the target antigen, expression or secretion of immune checkpoint molecules, or resistance to cytokines or cytotoxins; the characteristics of the target cells environment, such as acidity, hypoxia, and physical barriers, and others. Additionally, there are factors regarding therapy that can affect the response of CAR-T cells, such as concurrent immunotherapy, chemotherapy, radiation, and other medications that the subject may require. The effect of each of these parameters, factors, situations, and conditions can be examined using a library of the disclosure in a suitably designed experiment.

CAR-T cell experiments often use target cells in order to stimulate proliferation, differentiation, cytokine expression, cytotoxicity, and/or exhaustion. Target cells can be any type of cell capable of expressing the target antigen (the antigen against which the CAR is directed), and need not be viable. Model cell lines are often selected to simulate the cells to be targeted during treatment, for example using a lymphoma cell line to test CARs intended for treating lymphoma. The target cells can also be obtained from a subject to be treated, for example tumor cells or leukemia cells from a subject to be treated can be obtained, for example, by biopsy or blood draw respectively. Alternatively, CAR-T cells can be stimulated using beads having suitable antigens or antibodies bound to the surface, for example having anti-CD3 and anti-CD28 antibodies bound to the surface.
Proliferation

T cells expand clonally in response to antigen-specific stimulation through the T cell receptor, in combination with co-stimulation from other factors. Without being bound by any particular theory, it is believed that costimulation by proteins such as CD28 and 4-1BB are necessary for efficient clonal expansion, and that including the signaling domain(s) from such proteins in a CAR results in comparable activation and proliferation. This clonal expansion is a necessary part of the immune response, which insures that enough T cells are generated to combat the target cells as the number of target cells increases. However, T cells should not proliferate in the absence of such stimulation. The target cells are in general either normal cells infected with a virus or other intracellular pathogen, or are neoplastic or cancerous cells. Thus, proliferation is an important measure of CAR-T cell function and utility.

Proliferation can be measured by a number of different methods, depending on the type of library used. For example, where the library is a collection of different CAR-T clones (each clone having a different SD), each clone can be examined in individual cultures in parallel. In such cases, the number of CAR-T cells can be measured or estimated at times during the experiment and/or at the end of the experiment, for example without limitation by sorting and counting the cells (e.g., by FACS), and/or by quantifying the amount of signal from a marker protein such as green fluorescent protein (GFP), mCherry, mTomato, or the like. In the case of pooled libraries, CAR-T cells can also be sorted and counted in the same manner, if the library is constructed with a different marker protein for each different clone. This is practical as long as each marker protein can be distinguished from the other marker proteins in the sorting device, which generally limits such pooled libraries to about 4 to 7 different clones. For larger pooled libraries (e.g., pooled libraries including about 7 to about 60 different clones), other methods of counting are more practical, such as single cell sequencing. For such pooled libraries, the CAR nucleic acids can be provided with primer sequences that facilitate sequencing the SD and/or the entire receptor. In some embodiments, the library is a pooled library, and the proliferation of individual clones within the library is determined by single cell sequencing.
Exhaustion

T cells can become exhausted in response to constant or frequently repeated stimulation (e.g., loss of T cell function after repeated activation and/or antigen exposure), at which point they lose the ability to proliferate, express inflammatory cytokines, and kill target cells. Exhausted T cells can be characterized by the elevated expression of surface proteins such as TIM3, PD-1, LAG3, CTLA-4, CD39, CD43, and CD69, and reduced expression of proteins such as CD62L and CD127. It is accordingly a goal to identify CARs that help T cells to resist exhaustion.

Resistance to exhaustion can be measured in appropriately designed experiments, for example, by measuring the clonal expansion rate of a library (e.g., using the methods for measuring proliferation above) in response to constant stimulation for at least a period of time sufficient to cause exhaustion in normal T cells, and determining when (or if) the rate of proliferation declines or ceases. Libraries can also be subjected to cytokines that induce exhaustion, such as IL-10 or TGFβ, and assayed for any reduction in the rate or amount of exhaustion induced. Alternatively, libraries can be tested by sorting and measuring clones for expression of exhaustion markers, such as, for example, TIM3, PD-1, LAG3, CTLA-4, CD39, CD43 and CD69. In some embodiments, a library is subjected to conditions that can cause anergy, and is labeled for detection of one or more of the exhaustion markers TIM3, PD-1, LAG3, CTLA-4, CD39, CD43 and CD69. In some embodiments, the library is labeled for detection of 1, 2, 3, 4, 5, 6, or 7 exhaustion markers. Expression of one or more exhaustion markers can alternatively be measured by other means, for example using single cell RNA sequencing (scRNAseq). In some embodiments, a library is subjected to conditions that can cause anergy, and the expression of one or more of the exhaustion markers TIM3, PD-1, LAG3, CTLA-4, CD39, CD43 and CD69 is determined by scRNAseq. In some embodiments, the expression of 1, 2, 3, 4, 5, 6, or 7 exhaustion markers is determined by scRNAseq. In some embodiments, the library is an arrayed library. In some embodiments, the library is a pooled library. In some embodiments, the exhaustion marker is TIM3, PD-1, LAG3, or CD39. In some embodiments, the exhaustion markers are TIM3, PD-1, LAG3, and CD39.
Differentiation

After a T cell has been fully activated by exposure to antigen and the appropriate costimulatory factors, it may further differentiate into a memory T cell. A memory T cell can remain in the body for decades, and responds to the antigen it recognizes more quickly and strongly than a naïve T cell. Accordingly, it is useful to identify costimulatory domains that increase or accelerate differentiation of CAR-T cells into memory CAR-T cells.

Memory T cells are usually identified by the expression of characteristic surface protein markers, such as CD45RO. Different subclasses of memory T cells can also be identified by the expression of characteristic surface protein markers. Central memory T cells (T_{CM}, CD45RA⁻CD45RO⁺ CCR7⁺ CD62L⁺) are found mainly in lymph nodes and in the peripheral circulation. Effector memory (TEM, CD45RA⁻ CD45RO⁺ CCR7), and effector memory re-expressing CD45RA (TEMRA, CD45RA⁺ CD45RO⁺ CCR7⁻ ) cells are found mainly in the peripheral circulation and in tissues. Naïve T cells generally display CD45RA⁺ CD45RO⁻ CCR7⁺ antigens. In some embodiments, the library is stained for CD45RO expression after stimulation, and the number of CD45RO⁺ cells is quantified. In some embodiments, the library is examined for CD45RO expression by scRNAseq, and the number of CD45RO⁺ cells is quantified. In some embodiments, the library is stained for CD45RO expression after stimulation, and the number of CD45RO⁺ cells in a plurality of different clones is quantified. In some embodiments, the library is examined for CD45RO expression by scRNAseq, and the number of CD45RO⁺ cells in a plurality of different clones is quantified. In some embodiments, the library is examined for CD45RO expression at a number of time points after initial stimulation with antigen. In some embodiments, the library is examined at no less than 1, 2, 3, 4, 5, 6, 7, 8, 9, or 10 different time points after initial stimulation with antigen. In some embodiments, the library is examined at no more than 50, 40, 30, 20, 15, 14, 13, 12, 11, 10, 9, 8, 7, 6, or 5 different time points after initial stimulation with antigen. In some embodiments, the library is an arrayed library. In some embodiments, the library is a pooled library. Some particular CARs (e.g. BAFFR-containing CARs) may be benefitting from the prolonged expression of CD27, which is another important factor in memory T cell formation (www.jimmunol.org/content/180/5/2912.short).
Cytotoxicity

The ability to kill target cells is a primary function of T cells, particularly CD8⁺ T cells. CD4⁺ T cells can also exhibit direct cytotoxic and anti-viral effects through the secretion of perforin, granzyme B, and/or IFNγ. As set forth herein, different SDs can stimulate cytotoxicity to differing degrees. As with the other CAR parameters, one goal is to find SDs that highly increase antigen-induced cytotoxicity. Another goal is to find SDs that produce little or no tonic cytotoxicity (e.g., cytotoxicity in the absence of the target antigen). Another goal is to find SDs that simultaneously satisfy the first two goals.

Cytotoxicity can be measured using the standard protocols known in the art. In general, cytotoxicity is measured in an arrayed library, so that cytotoxicity can be attributed to the correct CAR. In general, living target cells are contacted with a library of engineered cells, followed by determination of target cell survival or death. Depending on the target cells, cytotoxicity may result in target cell death, or only a reduction in target cell proliferation (as compared to suitable controls). Target cell death can be quantified, for example, using a vital stain, which is excluded from target cells having an intact membrane, but enters damaged cells to stain intracellular structures. Examples of vital stains include Trypan Blue, 7AAD (7_amino actinomycin_D), propidium iodide, Zombie Green™ (and other Zombie stains, BioLegend, San Diego, CA). Alternatively, target cells can be transduced with a marker protein such as mKate, mCherry, GFP, and death of the target cells can be determined by reduction in the marker protein signal. Using such methods, cytotoxicity can be determined at one or more time points, or continuously. In some embodiments, the death of target cells is determined continuously.
Cytokines

CD4 T cells also respond to antigen stimulation by expressing and releasing cytokines such as interleukin-2 (IL-2), tumor necrosis factor-α (TNFα), and gamma interferon (IFNγ), which further increase immune functions. IL-2 induces T cells to differentiate into effector T cells and memory T cells. TNFα stimulates inflammatory responses. IFNγ has antiviral, antibacterial, and antifungal activity, and stimulates the differentiation of CD4+T_{H} cells into TH1 T cells. In some embodiments, a library of engineered cells is stimulated by contacting it with an antigen, and expression of at least one of IL-2, TNFα, and IFNγ is measured. In some embodiments, expression of two or more of IL-2, TNFα, and IFNγ is measured. In some embodiments, expression of IL-2, TNFα, and IFNγ is measured.

Cytokine expression can be measured directly, using commercially available immunoassays such as ELISA antibody-based kits. Intracellular cytokines can be stained by permeabilizing cells, and adding antibodies specific for the cytokines to be detected. If a pooled library is used, the cells can be sorted and identified (e.g., by single cell sequencing) after staining. Alternatively, cytokine expression can be measured using techniques such as scRNAseq. In some embodiments, a library of engineered cells is stimulated by contacting it with an antigen, and expression of at least one of IL-2, TNFα, and IFNγ is measured by immunoassay. In some embodiments, a library of engineered cells is stimulated by contacting it with an antigen, and expression of at least one of IL-2, TNFα, and IFNγ is measured by scRNAseq.
D. Methods of Treatment

Administration of any one of the therapeutic compositions described herein, e.g., chimeric receptors, nucleic acids, recombinant cells, and pharmaceutical compositions, can be used to treat subjects, e.g., patients for relevant diseases, such as proliferative diseases (e.g., cancers) and chronic infections. In some embodiments, the chimeric receptors, nucleic acids, recombinant cells, and pharmaceutical compositions described herein can be incorporated into therapeutic agents for use in methods of treating an individual who has, who is suspected of having, or who may be at high risk for developing one or more health conditions, such as proliferative diseases (e.g., cancers), autoimmune disorders, or diseases associated with checkpoint inhibition. Exemplary autoimmune disorders and diseases can include, without limitation, celiac disease, type 1 diabetes, Graves' disease, inflammatory bowel disease, multiple sclerosis, psoriasis, rheumatoid arthritis, and systemic lupus erythematosus.

Non-limiting exemplary embodiments of the methods of treating a health condition described herein can include one or more of the following features. In some embodiments, the health condition is a proliferative disease or an infection. Exemplary proliferative diseases can include, without limitation, angiogenic diseases, a metastatic diseases, tumorigenic diseases, neoplastic diseases and cancers. In some embodiments, the proliferative disease is a cancer.

Exemplary proliferative diseases can include, without limitation, angiogenic diseases, a metastatic diseases, tumorigenic diseases, neoplastic diseases and cancers. In some embodiments, the proliferative disease is a cancer. In some embodiments, the cancer is a pediatric cancer. In some embodiments, the cancer is a pancreatic cancer, a colon cancer, an ovarian cancer, a prostate cancer, a lung cancer, mesothelioma, a breast cancer, a urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, leukemia, lymphoma, and glioblastoma.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. In some embodiments, the administered composition inhibits tumor growth in the subject.

Accordingly, in one aspect, some embodiments of the disclosure relate to methods for modulating (e.g., inhibiting) an activity of a target cell in an individual, the methods include administering to the individual a first therapy including one or more of nucleic acids, recombinant cells, and pharmaceutical compositions as disclosed herein, wherein the first therapy modulates (e.g., inhibits) an activity of the target cell. In some embodiments, the administered nucleic acids, recombinant cells, and pharmaceutical compositions inhibit proliferation of a target cancer cell, and/or inhibits tumor growth of the cancer in the subject. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cells disclosed herein, wherein the recombinant cells inhibit an activity of the target cells in the individual. Generally, the target cells of the disclosed methods can be any cell type in an individual and can be, for example an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell. In some embodiments, the target cell is a pathogenic cell. In some embodiments, the target cell is a cancer cell. In some embodiments, the modulation of the activity the target cell results in the death of the target cell.

In another aspect, some embodiments of the disclosure relate to methods for the treatment of a health condition in an individual in need thereof, the methods include administering to the individual a first therapy including one or more of the recombinant cells comprising a chimeric receptor as disclosed herein, and/or pharmaceutical compositions as disclosed herein, wherein the first therapy treats the disease in the individual. In some embodiments, the methods include administering to the individual a first therapy including an effective number of the recombinant cells as disclosed herein, wherein the recombinant cells treat the disease.

In another aspect, some embodiments of the disclosure relate to methods for assisting in the treatment of a health condition in an individual in need thereof, the methods including administering to the individual a first therapy including one or more of chimeric receptors, nucleic acids, recombinant cells, and pharmaceutical compositions as disclosed herein, and a second therapy, wherein the first and second therapies together treat the disease in the individual. In some embodiments, the methods include administering to the individual a first therapy including an effective number of the recombinant cells as disclosed herein, wherein the recombinant cells treat the disease.

In another aspect, provided herein are methods for modulating the activity of an immune cell, the methods include: (a) expressing a chimeric receptor of the disclosure in the immune cell; and (b) contacting the immune cell with the target antigen. In some embodiments, the contacting is carried out in vivo, ex vivo, or in vitro. In some embodiments, the immune cell includes a chimeric receptor of the disclosure, and the activity of the immune cell is increased following the contacting as compared to the immune cell prior to the contacting or a corresponding immune cell that does not express the chimeric receptor. In some embodiments, the immune cell includes a chimeric receptor of the disclosure and the activity of the immune cell is decreased following the contacting as compared to the immune cell prior to the contacting or a corresponding immune cell that does not express the chimeric receptor.

In yet another aspect, some embodiments of the disclosure relate to methods for modulating an immune response to a first antigen in an individual, the methods include administering to the individual an effective amount of recombinant immune cells as disclosed herein.

Non-limiting exemplary embodiments of the disclosed methods for modulating an immune response can include one or more of the following features. In some embodiments, the recombinant immune cells include a chimeric receptor of the disclosure, the chimeric receptor is capable of binding to the first antigen, and an immune response to the first antigen is stimulated. In some embodiments, the recombinant immune cells include a chimeric receptor of the disclosure, the chimeric receptor is capable of binding to the first antigen, and an immune response to the first antigen is inhibited. In some embodiments, the recombinant immune cells include: (a) a chimeric receptor of the disclosure, wherein the chimeric receptor is activated by binding to a repressor antigen; and (b) a second receptor capable of binding to the first antigen to stimulate an immune response to the first antigen. In some embodiments, the first antigen is present on a target cell to which an immune response is desired and the repressor antigen is present on a non-target cell to which an immune response is not desired. In some embodiments, the methods further include administering to the individual an effective amount of the repressor antigen such that an immune response to the first antigen is inhibited.

In another aspect, provided herein are methods for modulating an immune response to an antigen in an individual, the methods include administering to the individual an effective amount of a pharmaceutical composition as disclosed herein such that the recombinant nucleic acid is introduced into immune cells in the individual capable of mediating the immune response. In some embodiments, the pharmaceutical composition includes a chimeric receptor as disclosed herein, wherein the chimeric receptor is capable of binding to the antigen, and an immune response to the antigen is stimulated. In some embodiments, the pharmaceutical composition includes a chimeric receptor as disclosed herein, wherein the chimeric receptor is capable of binding to the antigen, and an immune response to the antigen is inhibited.

In yet another aspect, provided herein are methods for treating a health condition in an individual in need thereof, including administering to the individual an effective amount of recombinant immune cells of the disclosure, wherein the recombinant immune cells treat the health condition. In some embodiments, the recombinant immune cells include a chimeric receptor of the disclosure, and the health condition is characterized by a pathogenic cell expressing the target antigen of the chimeric receptor. In some embodiments, the recombinant immune cells include (a) a chimeric receptor of the disclosure, wherein the chimeric receptor is activated by binding to a repressor antigen; and (b) a second receptor capable of binding to a second antigen associated with the health condition to stimulate an immune response to the second antigen. In some embodiments, the second antigen is present on a pathogenic cell associated with the health condition and the repressor antigen is present on a non-pathogenic cell. In some embodiments, the method further includes administering to the individual an effective amount of the repressor antigen such that an adverse effect in the individual mediated by the recombinant immune cells is inhibited.

### Administration of Recombinant Cells to an Individual

In some embodiments, the methods of the disclosure involve administering an effective amount or number of the recombinants cells to an individual in need of such treatment. This step can be accomplished using any method of implantation delivery in the art. For example, the recombinant cells can be infused or implanted directly in the individual's bloodstream or otherwise administered to the individual.

In some embodiments, the methods disclosed herein include administering, which term is used interchangeably with the terms “introducing,” implanting,” and “transplanting,” recombinant cells into an individual, by a method or route that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even the lifetime of the individual, e.g., long-term engraftment.

When provided prophylactically, the recombinant cells described herein can be administered to an individual in advance of any symptom of a disease or condition to be treated. Accordingly, in some embodiments the prophylactic administration of a recombinant cell population prevents the occurrence of symptoms of the disease or condition.

When provided therapeutically in some embodiments, recombinant cells are provided at (or after) the onset of a symptom or indication of a disease or condition, e.g., upon the onset of disease or condition.

For use in the various embodiments described herein, an effective amount or number of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors, or can be obtained from an autologous source. In some embodiments, the recombinant cells are expanded in culture prior to administration to an individual in need thereof.

In some embodiments, the delivery of a recombinant cell composition (e.g., a composition including a plurality of recombinant cells according to any of the cells described herein) into an individual by a method or route results in at least partial localization of the cell composition at a desired site. A composition including recombinant cells can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, and instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, intrasternal injection, and intrasternal infusion. In some embodiments, the route is intravenous. For the delivery of cells, delivery by injection or infusion is a standard mode of administration.

In some embodiments, the recombinant cells are administered systemically, e.g., via infusion or injection. For example, a population of recombinant cells are administered other than directly into a target site, tissue, or organ, such that it enters, the individual's circulatory system and, thus, is subject to metabolism and other similar biological processes.

The efficacy of a treatment including any of the compositions provided herein for the treatment of a disease or condition can be determined by a skilled clinician. However, one skilled in the art will appreciate that a treatment is considered effective if any one or all of the signs or symptoms or markers of disease are improved or ameliorated. In some embodiments, a sign or symptom or marker of a disease is ameliorated when the treatment is found to decrease, suppress, attenuate, diminish, arrest, or stabilize the development or progression of a disease or symptom or marker. Efficacy can also be measured by failure of an individual to worsen as assessed by decreased hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

As discussed above, a therapeutically effective amount includes an amount of a therapeutic composition that is sufficient to promote a particular beneficial effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some embodiments of the disclosed methods, the individual is a mammal. In some embodiments, the mammal is a human. In some embodiments, the individual has or is suspected of having a disease associated with inhibition of cell signaling mediated by a cell surface ligand or antigen. The diseases suitable for being treated by the compositions and methods of the disclosure include, but are not limited to, cancers, autoimmune diseases, inflammatory diseases, and infectious diseases. In some embodiments, the disease is a cancer or a chronic infection. In some embodiments, the infection is by a parasite. In some embodiments, the infection is by a parasite virus. In some embodiments, the infection is by a micro-fungus. In some embodiments, the infection is by a bacterium.

Methods for CAR design, delivery and expression in T cells, and the manufacturing of clinical-grade CAR-T cell populations are known in the art. See, for example, Lee et al., Clin Cancer Res (2012) 18(10):2780-90, hereby incorporated by reference in its entirety. For example, the engineered CARs may be introduced into T cells using retroviruses, which efficiently and stably integrate a nucleic acid sequence encoding the chimeric antigen receptor into the target cell genome. An exemplary method is described in the Examples section which follows.

Other methods known in the art include, but are not limited to, lentiviral transduction, transposon-based systems, direct RNA transfection, and CRISPR/Cas systems (e.g., type I, type II, or type III systems using a suitable Cas protein such Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, Cas12a (Cpf1), Cas13a (C2c2), Cas13b, Cas13d, CasF, CasG, CasH, Csy1, Csy2, Csy3, Cse1 (or CasA), Cse2 (or CasB), Cse3 (or CasE), CasX, CasY, Cse4 (or CasC), Csc1, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, Csx10, Csx16, CsaX, Csx3, Csz1, Csx15, Csfl, Csf2, Csf3, Csf4, and Cu1966, etc.

In some embodiments, a recombinant adeno-associated virus (AAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV requires that the following components are present within a single cell (denoted herein as a packaging cell): a rAAV genome, AAV rep and cap genes separate from (e.g., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Production of pseudotyped rAAV is disclosed in, for example, international patent application publication number WO 01/83692.

The CAR-T cells, once they have been expanded ex vivo in response to, for example, an autoimmune disease antigen, can be reinfused into the subject in a therapeutically effective amount.

The precise amount of CAR T cells to be administered can be determined by a physician with consideration of individual differences in age, weight, extent of disease and condition of the subject.

Administration of T cell therapies may be defined by number of total cells per infusion or number of cells per kilogram of body weight, especially for pediatric subjects (e.g., patients). As T cells replicate and expand after transfer, the administered cell dose may not resemble the final steady-state number of cells. In some embodiments, a pharmaceutical composition including the CAR T cells of the present disclosure may be administered at a dosage of 10⁴ to 10¹⁰ total cells. In another embodiment, a pharmaceutical composition including the CART cells of the present disclosure may be administered at a dosage of 10³ to 10⁸ cells/kg body weight, including all integer values within those ranges.

Compositions including the CAR T cells of the present disclosure may also be administered multiple times at these dosages. The cells can be administered by using infusion techniques that are known in the art (see, for example, Rosenberg et al., New Engl J Med, (1988) 319:1676). The optimal dosage and treatment regimen for a particular subject can be determined by one skilled in the art by monitoring the subject for signs of disease and adjusting the treatment accordingly.

In some embodiments, administration of any of the compositions embodied herein, for the treatment of, for example, an autoimmune or inflammatory disease, can be combined with other cell-based therapies, for example, stem cells, antigen presenting cells, pancreatic islets etc.

The composition of the present disclosure may be prepared in a manner known in the art and in a manner suitable for parenteral administration to mammals, particularly humans, including a therapeutically effective amount of the composition alone, with one or more pharmaceutically acceptable carriers or diluents.

The term “pharmaceutically acceptable carrier” as used herein means any suitable carriers, diluents or excipients. These include all aqueous and non-aqueous isotonic sterile injection solutions which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the present disclosure may also include other supplementary physiologically active agents.

The carrier must be pharmaceutically “acceptable” in the sense of being compatible with the other ingredients of the composition and not injurious to the subject. Compositions include those suitable for parenteral administration, including subcutaneous, intramuscular, intravenous and intradermal administration. The compositions may conveniently be presented in unit dosage form and may be prepared by any method well known in the art of pharmacy. Such methods include preparing the carrier for association with the CAR-T cells. In general, the compositions are prepared by uniformly and intimately bringing into association any active ingredients with liquid carriers.

In some embodiments, the composition is suitable for parenteral administration. In another embodiment, the composition is suitable for intravenous administration.

Compositions suitable for parenteral administration include aqueous and nonaqueous isotonic sterile injection solutions which may contain anti-oxidants, buffers, bactericides and solutes, which render the composition isotonic with the blood of the intended recipient; and aqueous and non-aqueous sterile suspensions which may include suspending agents and thickening agents.

### Additional Therapies

As discussed above, any one of the compositions as disclosed herein, e.g., the chimeric receptors, recombinant nucleic acids, recombinant cells, cell cultures, and pharmaceutical compositions described herein can be administered to a subject in need thereof as a single therapy (e.g., monotherapy). In addition or alternatively, in some embodiments of the disclosure, the chimeric receptors, recombinant nucleic acids, recombinant cells, cell cultures, and pharmaceutical compositions described herein can be administered to the subject in combination with one or more additional therapies, e.g., at least one, two, three, four, or five additional therapies. Suitable therapies to be administered in combination with the compositions of the disclosure include, but are not limited to chemotherapy, radiotherapy, immunotherapy, hormonal therapy, toxin therapy, targeted therapy, and surgery. Other suitable therapies include therapeutic agents such as chemotherapeutics, anti-cancer agents, and anti-cancer therapies.

Administration “in combination with” one or more additional therapies includes simultaneous (concurrent) and consecutive administration in any order. In some embodiments, the one or more additional therapies is selected from the group consisting of chemotherapy, radiotherapy, immunotherapy, hormonal therapy, toxin therapy, and surgery. The term chemotherapy as used herein encompasses anti-cancer agents. Various classes of anti-cancer agents can be suitably used for the methods disclosed herein. Non-limiting examples of anti-cancer agents include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

The present disclosure also contemplates the combination of the composition of the disclosure with other drugs and/or in addition to other treatment regimens or modalities such as surgery. When the composition of the present disclosure is used in combination with known therapeutic agents the combination may be administered either in sequence (either continuously or broken up by periods of no treatment) or concurrently or as an admixture. In the case of, for example, autoimmune diseases, treatment includes administering to the subject the compositions embodied herein, e.g. autologous T cells transduced or contacted with a CAR embodied herein and one or more anti-inflammatory agents and/or therapeutic agents. The anti-inflammatory agents include one or more antibodies which specifically bind to pro-inflammatory cytokines, e.g., pro-inflammatory cytokines such as IL-1, TNF, IL-6, GM-CSF, and IFN-γ. In some embodiments, the antibodies are anti-TNFα, anti-IL-6 or combinations thereof. In some embodiments, one or more agents, other than antibodies can be administered which decrease pro-inflammatory cytokines, e.g. non-steroidal anti-inflammatory drugs (NSAIDs). Any combination of antibodies and one or more agents can be administered which decrease pro-inflammatory cytokines.

Treatment in combination is also contemplated to encompass the treatment with either the composition of the disclosure followed by a known treatment, or treatment with a known agent followed by treatment with the composition of the disclosure, for example, as maintenance therapy. For example, in the treatment of autoimmune diseases, excessive and prolonged activation of immune cells, such as T and B lymphocytes, and overexpression of the master pro-inflammatory cytokine tumor necrosis factor alpha (TNF), together with other mediators such as interleukin-6 (IL-6), interleukin-1 (IL-1), and interferon gamma (IFN-y), play a central role in the pathogenesis of autoimmune inflammatory responses in rheumatoid arthritis (RA), inflammatory bowel disease (IBD), Crohn's disease (CD), and ankylosing spondylitis (AS).

Non-steroidal anti-inflammatory drugs (NSAIDs), glucocorticoids, disease-modifying anti-rheumatic drugs (DMARDs) are traditionally used in the treatment of autoimmune inflammatory diseases. NSAIDs and glucocorticoids are effective in the alleviation of pain and inhibition of inflammation, while DMARDs have the capacity of reducing tissue and organ damage caused by inflammatory responses. More recently, treatment for RA and other autoimmune diseases has been revolutionized with the discovery that TNF is critically important in the development of the diseases. Anti-TNF biologics (such as infliximab, adalimumab, etanercept, golimumab, and certolizumab pepol) have markedly improved the outcome of the management of autoimmune inflammatory diseases.

Non-steroidal anti-inflammatory drugs have the analgesic, antipyretic, and anti-inflammatory effect, frequently used for the treatment of conditions like arthritis and headaches. NSAIDs relieve pain through blocking cyclooxygenase (COX) enzymes. COX promotes the production of prostaglandins, a mediator which causes inflammation and pain. Although NSAIDs have different chemical structures, all of them have the similar therapeutic effect, e.g., inhibition of autoimmune inflammatory responses. In general, NSAIDs can be divided into two broad categories: traditional non-selective NSAIDs and selective cyclooxygenase-2 (COX-2) inhibitors (For a review, see, P. Li et al., Front Pharmacol (2017) 8:460).

In addition to anti-TNF agents, the biologics targeting other proinflammatory cytokines or immune competent molecules have also been extensively studied and actively developed. For example, abatacept, a fully humanized fusion protein of extracellular domain of CTLA-4 and Fc fraction of IgG1, has been approved for the RA patients with inadequate response to anti-TNF therapy. The major immunological mechanism of abatacept is selective inhibition of co-stimulation pathway (CD80 and CD86) and activation of T cells. Tocilizumab, a humanized anti-IL-6 receptor monoclonal antibody was approved for RA patients intolerant to DMARDs and/or anti-TNF biologics. This therapeutic mAb blocks the transmembrane signaling of IL-6 through binding with soluble and membrane forms of IL-6 receptor. Biological drugs targeting IL-1 (anakinra), Thl immune responses (IL-12/IL-23, ustekinumab), Th17 immune responses (IL-17, secukinumab) and CD20 (rituximab) have also been approved for the treatment of autoimmune diseases (For a review see, P. Li et al., Front Pharmacol (2017) 8:460).

Accordingly, in some embodiments, the methods of the disclosure include administration of a composition disclosed herein to a subject individually as a single therapy (e.g., monotherapy). In some embodiments, a composition of the disclosure is administered to a subject as a first therapy in combination with a second therapy. In some embodiments, the second therapy is selected from the group consisting of chemotherapy, radiotherapy, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.
E. Methods for Isolation of Cells

Any number of methods known in the art can be used to isolate cells, for transduction with any number of chimeric receptors (e.g., CARs) described herein, such as Tregs, or any other cell type that may be used in carrying out the treatment of a subject. Thus, also provided are various other genetically engineered cells expressing the chimeric receptors, e.g., CARs. The cells generally are eukaryotic cells, such as mammalian cells, such as human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, such as T cells and/or NK cells. Other exemplary cells include stem cells, such as multipotent and pluripotent stem cells, including induced pluripotent stem cells (iPSCs). In some embodiments, the cells are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods of isolating cells suitable for the disclosure include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same subject, before or after cryopreservation.

In some embodiments, the cells include one or more nucleic acids introduced via genetic engineering, and thereby express recombinant or genetically engineered products of such nucleic acids. In some embodiments, the nucleic acids are heterologous, e.g., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one including chimeric combinations of nucleic acids encoding various domains from multiple different cell types.

In some embodiments, preparation of the engineered cells includes one or more culture and/or preparation steps. The cells for introduction of the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. In some embodiments, a biological sample is obtained from one or more sources including: autologous, allogeneic, haplotype matched, haplotype mismatched, haplo-identical, xenogeneic, cell lines or combinations thereof.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In some embodiments, a cell population described herein is collected and enriched (or depleted) by use of microelectromechanical systems (MEMS) chips in combination with a FACS-based detection system (see, e.g., WO 2010/033140; Cho et al. Lab Chip (2010) 10:1567-73; and Godin et al. J Biophoton (2008) 1(5):355-76. In both cases, cells can be labeled with multiple markers, allowing for the isolation of well-defined T cell subsets at high purity.

In some embodiments, the stimulating conditions or agents include one or more agent, e.g., ligand, which is capable of activating an intracellular SD of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further include the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al.; Klebanoff et al., J Immunother (2012) 35(9):651-60, Terakura et al., Blood (2012) 1:72-82, and/or Wang et al., J Immunother (2012) 35(9):689-701.
F. Systems and Kits

Also provided herein are systems and kits including the chimeric receptors, recombinant nucleic acids, recombinant cells, or pharmaceutical compositions provided and described herein as well as written instructions for making and using the same. For example, provided herein, in some embodiments, are systems and/or kits that include one or more of the following: a chimeric receptor as described herein, a recombinant nucleic acid as described herein, a recombinant cell as described herein, or a pharmaceutical composition as described herein.

In some embodiments, the systems and/or kits of the disclosure further include one or more syringes (including pre-filled syringes) and/or catheters (including pre-filled syringes) used to administer one any of the provided chimeric receptors, recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cancer cell, or treating a disease in an individual in need thereof

Any of the above-described systems and kits can further include one or more additional reagents, where such additional reagents can be selected from: dilution buffers, reconstitution solutions, wash buffers, control reagents, control expression vectors, negative control polypeptides, positive control polypeptides, reagents for in vitro production of the chimeric receptor polypeptides.

In some embodiments, the components of a system or kit can be in separate containers. In some other embodiments, the components of a system or kit can be combined in a single container.

In some embodiments, a system or kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

Also provided herein are systems and kits including the chimeric receptors, recombinant nucleic acids, recombinant cells, or pharmaceutical compositions provided and described herein as well as written instructions for making and using the same. For example, provided herein, in some embodiments, are systems and/or kits that include one or more of the following: a chimeric receptor as described herein, a recombinant nucleic acid as described herein, a recombinant cell as described herein, or a pharmaceutical composition as described herein.

In some embodiments, the systems and/or kits of the disclosure further include one or more syringes (including pre-filled syringes) and/or catheters (including pre-filled syringes) used to administer one any of the provided chimeric receptors, recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cancer cell, or treating a disease in an individual in need thereof.

Any of the above-described systems and kits can further include one or more additional reagents, where such additional reagents can be selected from: dilution buffers, reconstitution solutions, wash buffers, control reagents, control expression vectors, negative control polypeptides, positive control polypeptides, reagents for in vitro production of the chimeric receptor polypeptides.

In some embodiments, the components of a system or kit can be in separate containers. In some other embodiments, the components of a system or kit can be combined in a single container.

In some embodiments, a system or kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferre, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014), and Makrides, S. C. (2003). Gene Transfer and Expression in Mammalian Cells. Amsterdam, NL: Elsevier Sciences B.V., the disclosures of which are incorporated herein by reference.

### Example 1

### CAR-T Library Construction

As discussed above, it was hypothesized that CR-mediated signaling is determined by the identity of its SDs and has a measurable impact on effector function of immune cells expressing the CR. Accordingly, modifying the signaling component of a CR by introducing one or more endodomains significantly impacts CR-mediated signaling. In the experiments described below, CR SDs were designed herein to effect one or more immunomodulatory functions, such as co-stimulatory or inhibitory functions.

In these experiments, intracellular signaling domains (SDs) were synthesized as gBlock gene fragments (Integrated DNA Technologies). The SDs used were derived from the proteins listed in Table 1 below.

TABLE 1
Summary of exemplary SD domains used in the present disclosure.

| | Amino acid | Nucleic acid |
|---|---|---|
| SD Source | SEQ ID NO: | SEQ ID NO: |
| 2B4 | 20 | 67 |
| 4-1BB (CD137) | 4 | 51 |
| Allergin-1 | 44 | 91 |
| B7-1 (CD80) | 102 | 103 |
| B7-2 (CD86) | 116 | 117 |
| BAFF-R | 24 | 71 |
| BCMA | 26 | 73 |
| BTLA | 18 | 65 |
| CD2 | 19 | 66 |
| CD200R | 40 | 87 |
| CD244 | 118 | 119 |
| CD226 | 9 | 56 |
| CD27 | 6 | 53 |
| CD28 | 5 | 52 |
| CD30 (TNR8) | 21 | 68 |
| CD300a | 41 | 88 |
| CD300f | 42 | 89 |
| CD40 | 10 | 57 |
| CD7 | 3 | 50 |
| CD72 | 27 | 74 |
| CD96 | 28 | 75 |
| CRACC | 30 | 77 |
| CRTAM | 13 | 60 |
| CTLA4 | 2 | 49 |
| CXADR | 17 | 64 |
| DC-SIGN | 43 | 90 |
| DR6 (TNFRSF21) | 104 | 105 |
| GITR | 8 | 55 |
| HAVCR2 | 120 | 121 |
| ICOS | 1 | 48 |
| ILT2 | 34 | 81 |
| ILT3 | 37 | 84 |
| ILT4 | 38 | 85 |
| KIR2DL1 | 35 | 82 |
| KIR3DL1 | 36 | 83 |
| KLRG1 | 32 | 79 |
| LAG3 | 7 | 54 |
| LAIR1 | 16 | 63 |
| LY9 | 106 | 107 |
| NGFR | 108 | 109 |
| NKG2D | 22 | 69 |
| NKR-P1A | 33 | 80 |
| NTB-A | 29 | 76 |
| OX40 (CD134, TNFRSR4) | 110 | 111 |
| PD-1 | 14 | 61 |
| PIR-B | 45 | 92 |
| RELT (TNFRSF19L) | 112 | 113 |
| SIGLEC-3.9.9 | 31 | 78 |
| TACI | 25 | 72 |
| TIGIT | 12 | 59 |
| TIM-1 | 23 | 70 |
| TIM-3 | 11 | 58 |
| TLT-1 | 39 | 86 |
| TNFRSF25 | 15 | 62 |
| TROY (TNFRSF19) | 114 | 115 |

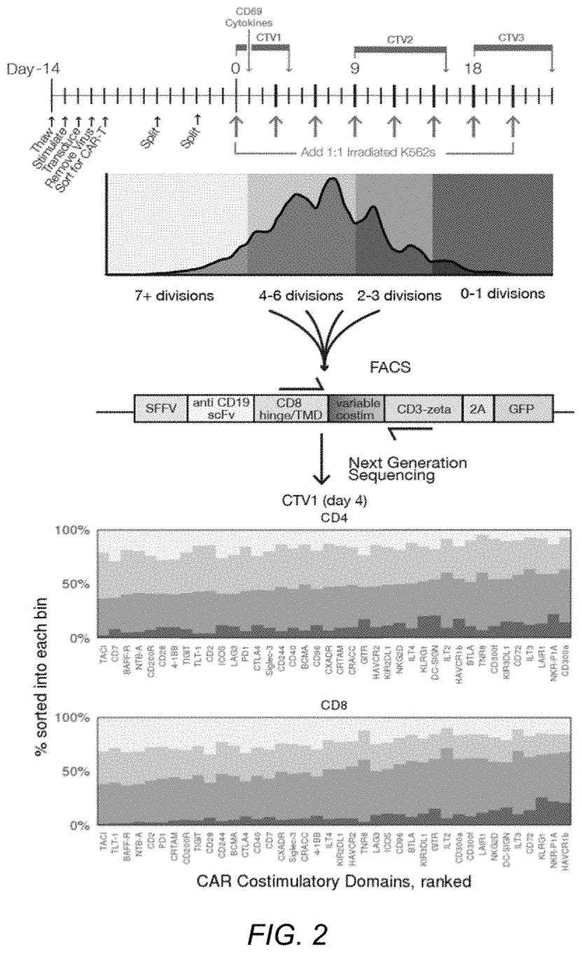

These fragments were amplified with common primers having homology to an E. coli cloning backbone, and the PCR products were individually cloned into the backbone in 96-well plates. Colonies were sequence verified after cloning using Sanger sequencing. Each cloned costimulatory domain was cultured and mini-prepped separately, and then pooled at a 1:1 molar ratio. A fragment containing a silencing-prone spleen focus forming virus (SFFV) promoter and N-terminal portion of the CAR having an anti-CD19 scFv (SEQ ID NO: 100) and a CD8 hinge/transmembrane domain (SEQ ID NO: 96) up to the costimulatory domain was inserted in front of the pooled SD plasmid library using Golden Gate Assembly and electroporation. This completed CAR cloning plasmid library was then digested to separate the promoter, CAR, and a downstream GFP marker from the backbone. A schematic diagram of the constructs is depicted in FIG. 2. The digested plasmid library was finally gel-extracted and inserted via restriction cloning and electroporation into a digested and gel-extracted pHRSIN lentiviral backbone. To ensure proper coverage of the library, with at least 1000 individual colonies per library member, on average, at each pooled electroporation step, the cloning procedure was optimized for the number of colonies at each cloning step. Parameters optimized included DNA cleanup procedure and restriction assembly parameters, including DNA volumes, enzyme volumes and ligation/restriction reaction timing, and cellular growth conditions.

Primary CD4⁺ and CD8⁺ T cells were isolated from Leukopaks (STEMCELL Technologies Cat #70500), which are an enriched leukapheresis product consisting of a large number of single-donor mononuclear cells (MNCs) collected from normal peripheral blood. T cells were cryopreserved in growth medium (RPMI-1640, UCSF cell culture core) with 20% human AB serum (Valley Biomedical Inc., #HP1022) and 10% DMSO. After thawing, T cells were cultured in human T cell medium containing X-VIVO™ 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments.

The second-generation CD19 CAR library constructs containing CD3 were introduced into cells via lentiviral transduction to provide a library of CAR-T cells. Lentiviral vectors pseudo-typed with vesicular stomatitis virus envelope G protein (VSV-G) (pantropic vectors) were produced via transfection of Lenti-X™ 293T cells (Clontech #11131D) with a pHR'SIN:CSW transgene expression vector and the viral packaging plasmids pCMVdR8.91 and pMD2. G using Mirus TranslT®-Lenti #MIR 6606). Primary T cells were thawed the same day and, after 24 hours in culture, were stimulated with beads having anti-CD3 and anti-CD28 antibodies bound to the surface (Human T-Activator CD3/CD28 Dynabeads®, Life Technologies #11131D) at a 1:3 cell:bead ratio. At 48 hours, viral supernatant was harvested and the primary T cells were contacted with the virus for 24 hours. At Day 5 post T cell stimulation, the beads were removed and the T cells were sorted for assays with a Beckton Dickinson (BD) FACSAria™ II flow cytometer, and the T cells expanded until Day 14 when they were rested and could be used in assays.

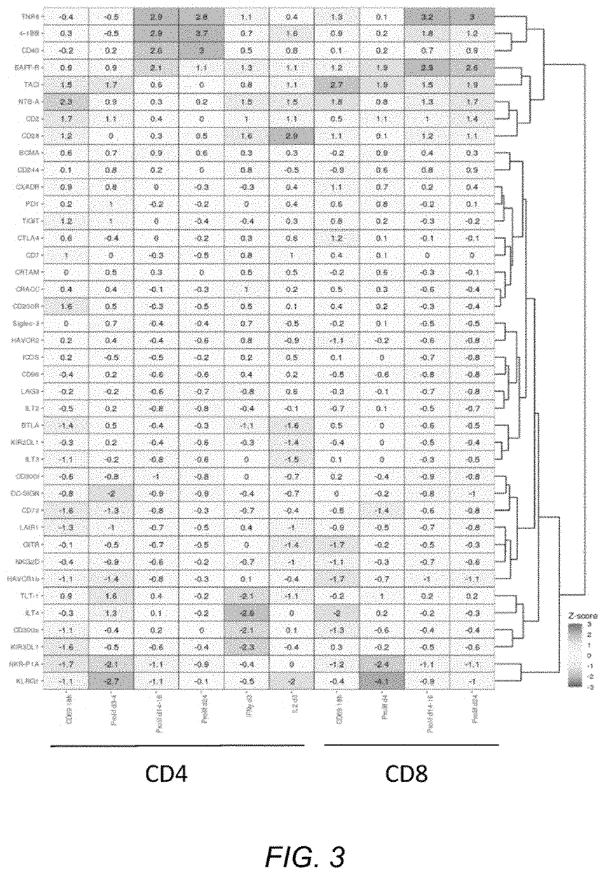

FIG. 3 depicts a hierarchical clustering of CARs (y axis) vs Pooled Assays (x axis). Each pooled assay was performed as indicated in the schematic in FIG. 2 and assessed using FACS based on level of protein expression and subsequent next-generation sequencing (NGS). In this analysis, colors and values are the z-scores for each assay; CARs are clustered and ranked from most activated//proliferative/highest cytokine production to least. Z-scores are calculated by combining scaled values within each donor and replicate. Different CARs perform better at different timepoints (early vs late) and in CD4s vs CD8s (see, e.g., table/heatmap). To determine the degree of activation of each CAR, antigen-naïve T cells were stimulated with K562 cells, either with or without CD19 expression, at a 1:1 ratio. 24 hours following plating of the co-culture the cells were collected by centrifugation at 500×g for 5 minutes, washed twice with flow buffer (PBS+2% FBS), and stained with anti-CD69 antibodies at 4° C. for 20 minutes. The cells were washed twice with flow buffer and analyzed by flow cytometry. To determine the degree of cytokine production of each CAR, antigen-naïve pooled library T cells were stimulated as described above for 18 hours before the addition of 2× Brefeldin A (Biolegend #420601) for an additional 6 hours in culture. Cells were collected by centrifugation at 500×g for 5 minutes, washed twice in flow buffer (PBS+2% FBS), and stained with anti-CD4 or -CD8 antibodies at 4° C. for 20 minutes. The cells were washed twice with flow buffer and 100 μL of fixative (50 μL of flow buffer+50 μL of Invitrogen IC fix) was added to each well. The samples were incubated at room temperature for 1 hour in the dark. After fixation the cells were spun at 600×g for 5 minutes and resuspended in Cytolast for continued staining the next day. To permeabilize the cells, 200 μL of 1× Permeabilization buffer was added to each well, and cells were immediately spun at 600×g for 5 minutes, and stained for intracellular antigens with anti-IL-2, -IL4, and -IFNy antibodies diluted in permeabilization buffer. The cells were stained in a 50 μL reaction at room temperature for 30 minutes in the dark. The cells were then washed twice with permeabilization buffer and analyzed by flow cytometry.

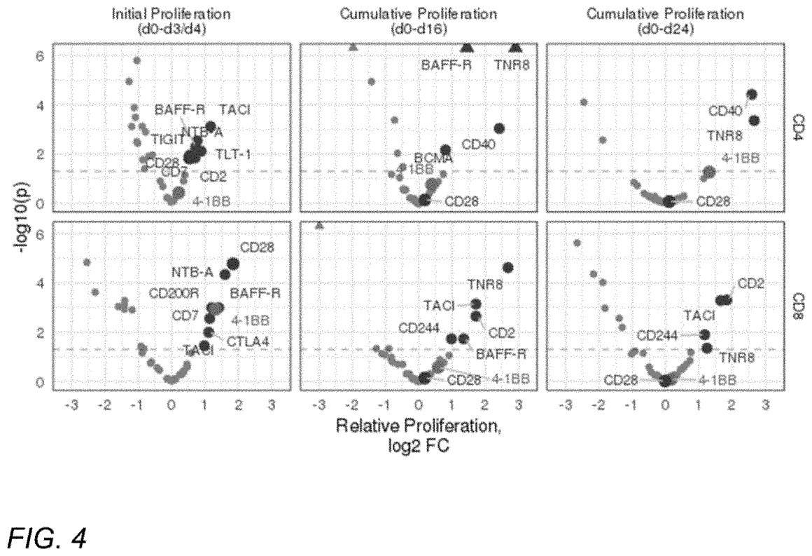

Subsequently, CAR performance were assessed. Depicted in FIG. 4 is volcano plot of the CAR performance across different assay times and T cell subsets. The leftmost bin in this figure was compared to the two rightmost bins to show estimated fold-change in abundance between the sorted populations, and associated with an adjusted p-value based on a count-adjusted negative binomial model. CAR costimulatory domains whose difference is significant in the assay across multiple donors are shown as larger points, while the benchmark costimulatory domains 4-1BB and CD28 are shown in shades of blue.

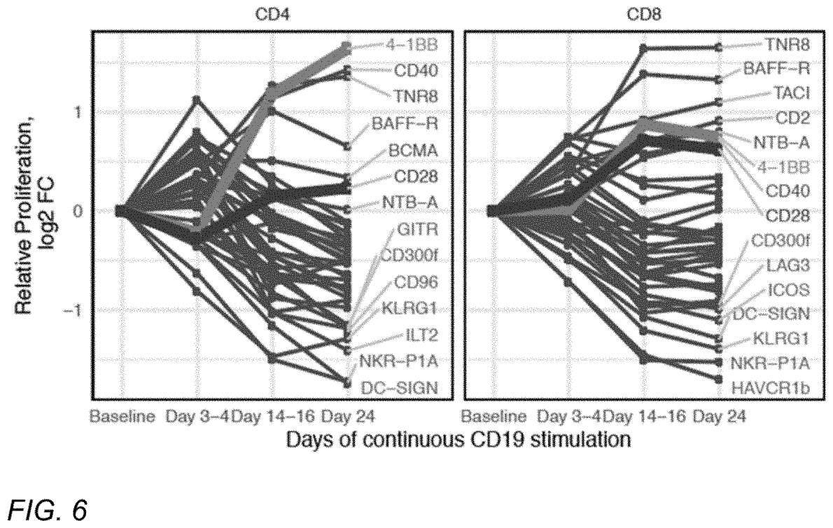

The results of experiments illustrating abundance change over time for the CARs is summarized in FIG. 6, wherein Y axis shows the change over time in relative abundance compared from the relative abundance prior to stimulation. In this figures, the top and bottom six CARs are shown, along with CD28 and 4-1BB control CARs. These data are averaged for each CAR across all donors and replicates, and between CD4 and CD8 T cell experiments.

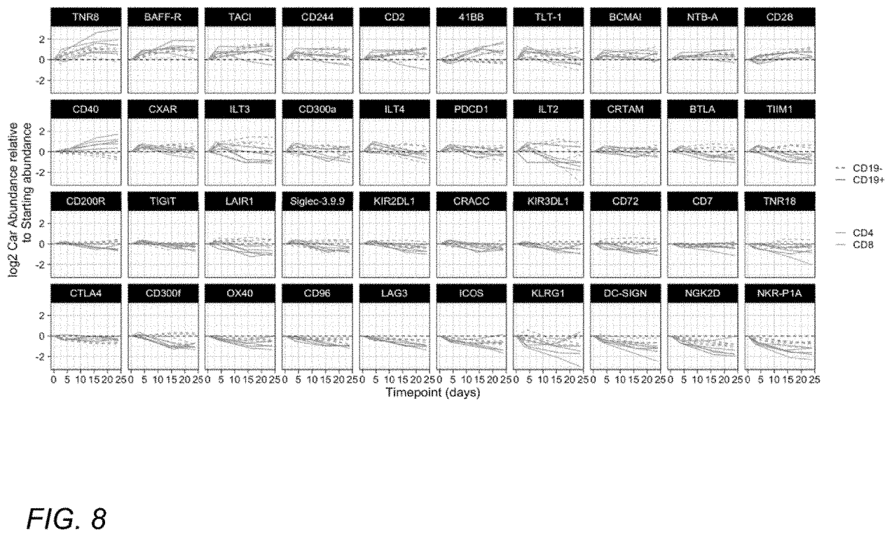

FIG. 8 shows a similar change in abundance over time as in FIG. 6, but for all CARs, with the results of pooled experiments to illustrate abundance change over time for the expanded set of CARs, separately showing lines for individual experimental replicates, CD4s, CD8s, as well as antigen-positive and antigen-negative conditions.

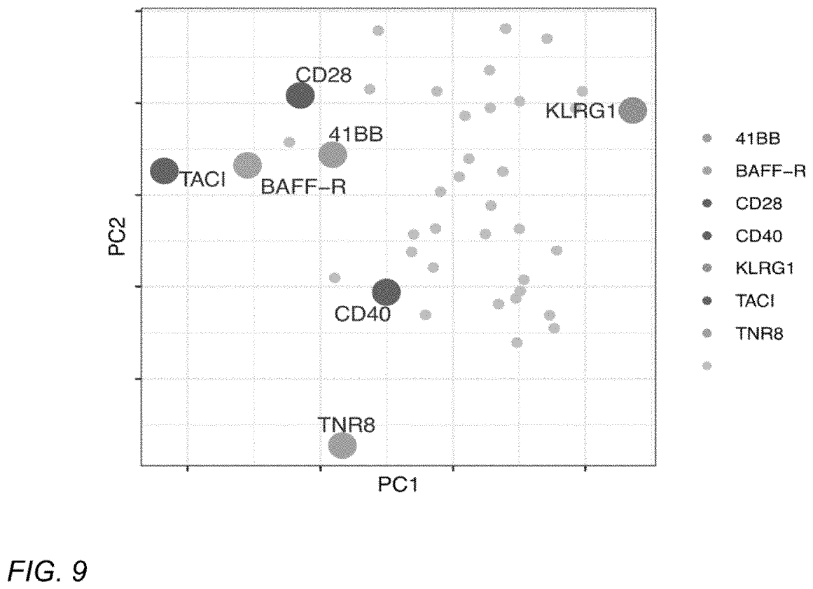

FIG. 9 depicts PCA plot of CAR performance across all assays and identification of 5 CARs for arrayed follow up. All metrics for each CAR were gathered across all donors, replicates, and stimulation conditions. Metrics included average-fold change in abundance at each time point as well as flow-cytometric proliferation and cytokine measurements. In subsequent arrayed screens, the focus was placed on the new receptors in with large labelled points, including the negative control receptor, in red.

### Example 2

### CAR-T Proliferation and Activation

CAR T Cell Proliferation

The cancer cell lines used as stimulus and targets were K562 myelogenous leukemia cells (ATCC #CCL-243). K562 cells were lentivirally transduced to stably express human CD19 at equivalent levels as Daudi tumors. CD19 levels were determined by staining the cells with α-CD19 APC (Biolegend #302212). All cell lines were sorted for expression of the transgene.

T cell proliferation was measured using a cell trace violet (CTV) assay. Cells were taken from culture, resuspended, and washed with PBS. The culture was resuspended to 10⁶ cells/mL in a 5 μM solution of CTV in PBS, followed by incubation at room temperature for twenty minutes in the dark. Next, 5 mL of T cell media was added on top for every 10⁶ cells that were stained. The cells were incubated for another 10 minutes in the dark, and then pelleted via centrifugation at 500×g for 5 minutes. The cells were then resuspended and plated in T cell media at 10⁶ cells/mL. To stimulate proliferation, an equal number of K562 cells relative to the total number of T cells were plated in each well, with or without CD19 expression, at a final concentration of 0.5×10⁶ K562 cells/mL and 0.5×10⁶ T cells/mL. The proliferation of the T cells was assessed 3 days after stimulation for CD4+ T cells via flow cytometry. The CD8+ T cells were then re-stimulated with an additional dose of K562 cells 3 days after the primary stimulation as described in the method below. Proliferation was then measured one day after the secondary stimulation via flow cytometry.

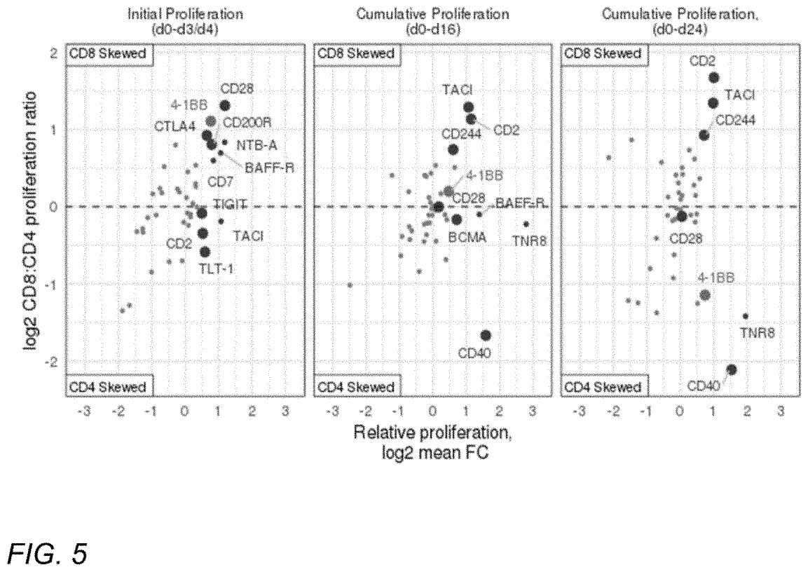

FIG. 5 shows a comparison of CD4 and CD8 T cell proliferation across the time points. Proliferation in CD4s and CD8s are compared, using the same analysis and features as described in the previous figure. The Y axis is the relative log2 ratio of CD8 to CD4 proliferation, while the X axis shows mean relative proliferation of both CD4s and CD8s. Proliferation is generally stronger/more differential in CD8s at early timepoints. At later timepoints, some CARs (CD40, TNR8, 4-1BB), expand preferentially in CD4s at later times, while others (CD2, TACI, CD244) expand preferentially in CD8s.

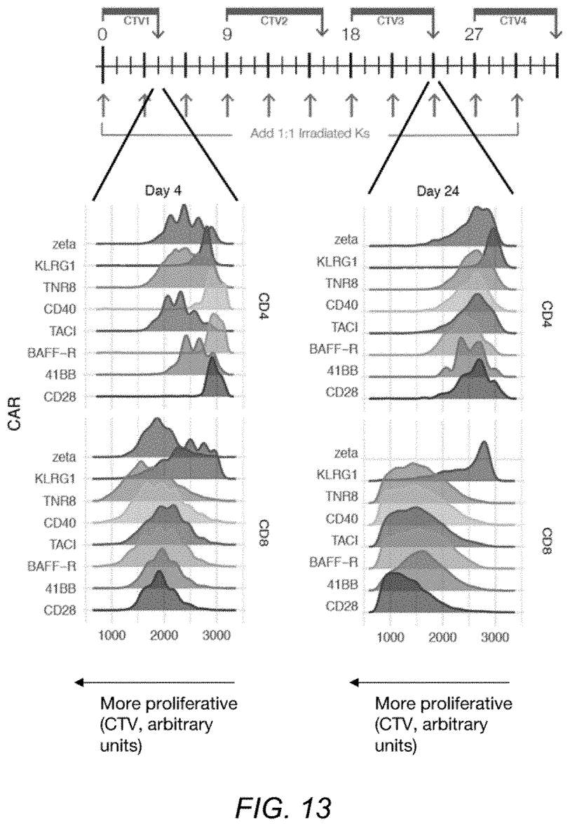

FIG. 13 graphically summarizes the results of experiments performed to illustrate that differential costimulatory domains alter proliferation dynamics. In these experiments, primary human CD4 and CD8 T cells were lentivirally transduced with each CAR, sorted for purity, and stimulated with CD19+ or CD19− irradiated K562 tumor cells every three days over a period of 33 days. 4 Cell Trace Violet (CTV) stains were assayed using flow cytometry at various times shown in the schematic at the top, and a representative donor is shown at Day 4 (CTV1), and Day 24 (CTV3), comparing CAR proliferation (lower is more proliferative).

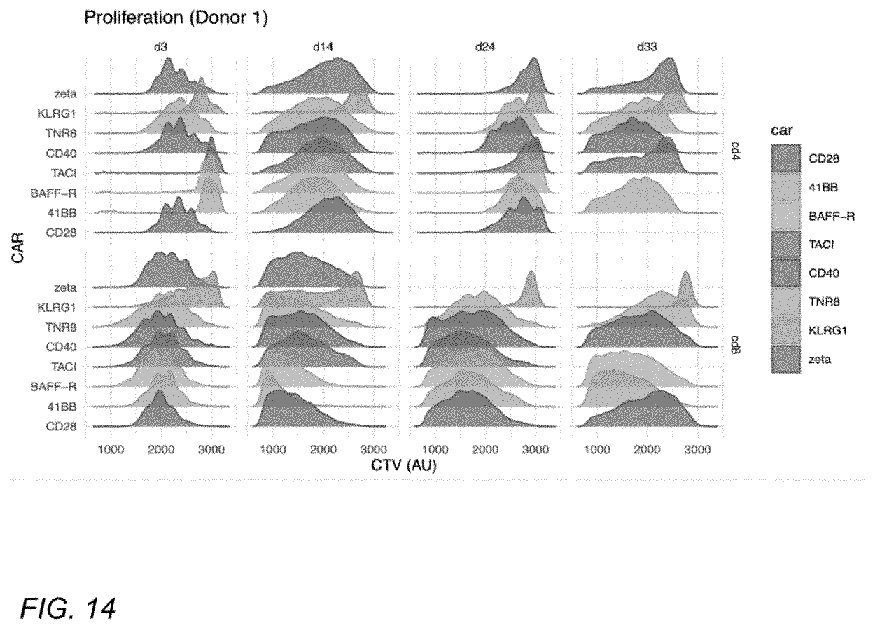

FIG. 14 graphically depicts the results of experiments performed to illustrate that differential costimulatory domains alter proliferation dynamics. In these experiments, four (4) CTV stains were assayed using flow cytometry at various times as shown in the schematic at the top of the previous figure. In these experiments, a single donor (Donor 1) is shown for all 4 time points across all 8 assayed CARs in CD4s and CD8s.

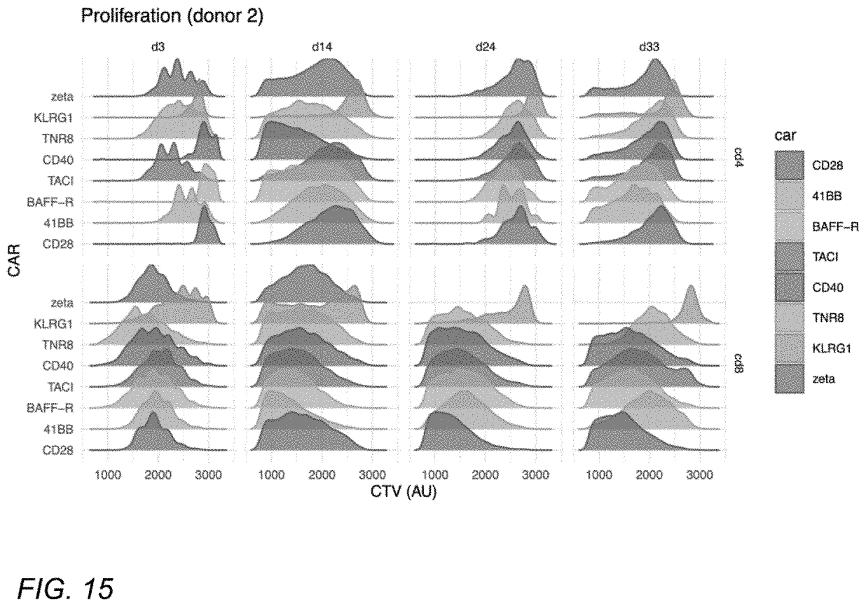

FIG. 15 graphically depicts the results of additional experiments performed to illustrate that differential costimulatory domains alter proliferation dynamics. Similarly to experiments described in FIG. 14, 4 CTV stains were assayed using flow cytometry at various times as shown in the schematic at the top of FIG. 13. In these experiments, a single donor (Donor 2) is shown for all 4 time points across all 8 assayed CARs in CD4s and CD8s.

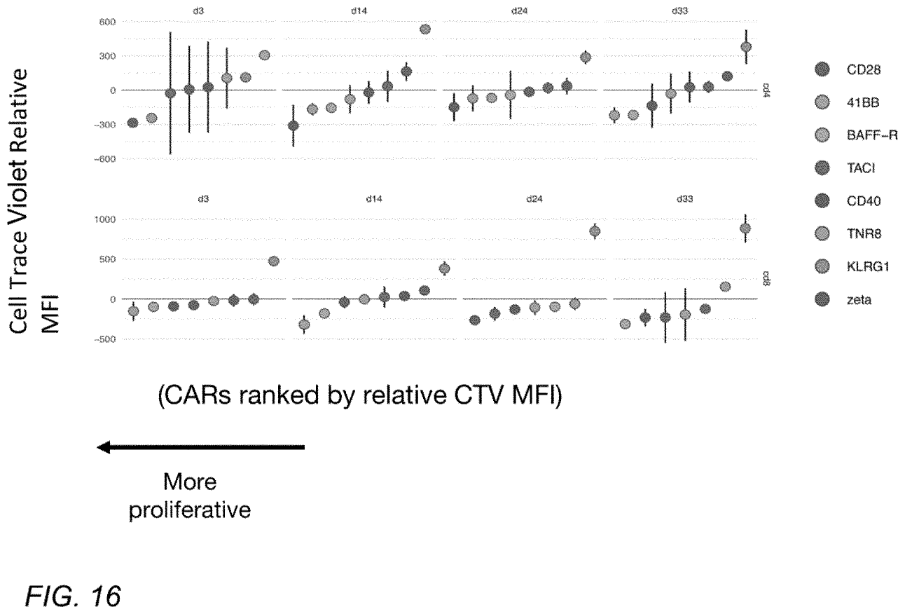

FIG. 16 summarizes the results of additional experiments performed to illustrate that differential costimulatory domains alter proliferation dynamics. These experiments were described in FIG. 13. Here both donors are combined into a relative mean proliferation for each time point for CD4s and CD8s separately. Individual CARs are ranked for each time point, with more proliferative CARs shown on the left-hand side of each subpanel.
CAR T Cell Activation

To determine the degree of activation of each CAR, antigen-naïve T cells were stimulated with K562 cells, either with or without CD19 expression, at a 1:1 ratio. 24 hours following plating of the co-culture the cells were collected by centrifugation at 500×g for 5 minutes, washed twice with flow buffer (PBS+2% FBS), and stained with anti-CD69 antibodies at 4° C. for 20 minutes. The cells were washed twice with flow buffer and analyzed by flow cytometry.

To determine the degree of cytokine production upon activation of each CAR, antigen-naïve T cells were stimulated with K562 cells, either with or without CD19 expression, at a 1:1 ratio. 3 days after plating of the co-culture a secondary bolus of K562 cells was added (see the above method). 2x Brefeldin A was added 12 hours later for an additional 6 hours. Cells were collected by centrifugation at 500×g for 5 minutes, washed twice in flow buffer (PBS+2% FBS), and stained with anti-CD4 or -CD8 antibodies at 4° C. for 20 minutes. The cells were washed twice with flow buffer and 100 μL of fixative (50 μL of flow buffer+50 μL of Invitrogen IC fix) was added to each well. The samples were incubated at room temperature for 1 hour in the dark. After fixation, the cells were spun at 600×g for 5 minutes and resuspended in Cytolast for continued staining the next day. To permeabilize the cells, 200 μL of 1× Permeabilization buffer was added to each well, and cells were immediately spun at 600×g for 5 minutes, and stained for intracellular antigens with anti-IL-2, -TNFα, and -IFNγ antibodies diluted in permeabilization buffer. The cells were stained in a 504 reaction at room temperature for 30 minutes in the dark. The cells were then washed twice with permeabilization buffer and analyzed by flow cytometry.

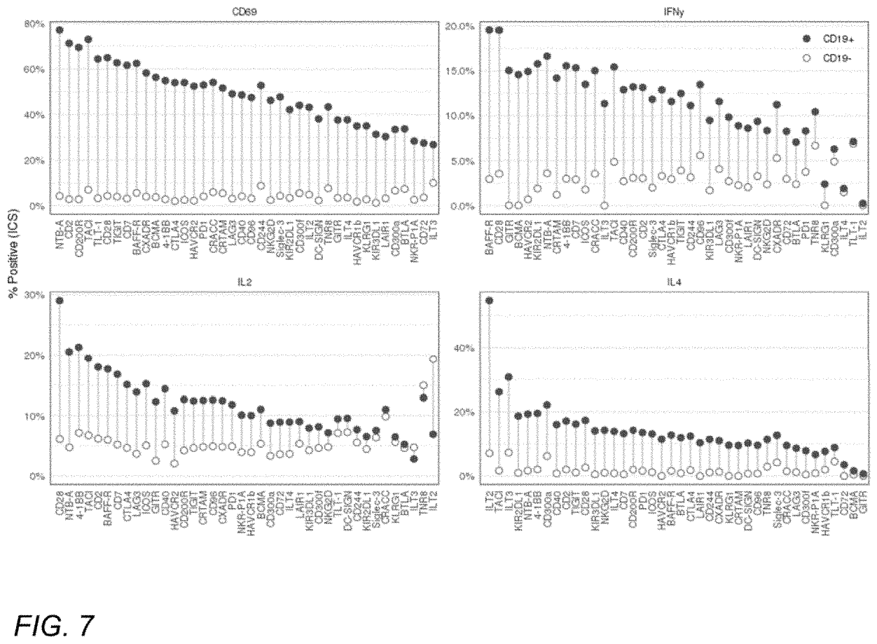

FIG. 7 graphically summarizes the results of experiments to illustrate relative activation and cytokine secretion upon initial stimulation with target cells, showing pooled sorting-based analysis of activation and cytokine production via surface and intracellular staining. Open circles are from co-culture with target cells not expressing CD19 antigen, and closed circles from co-culture with target cells that do express CD19 antigen. CD28 strongly secretes IL-2, ILT2 is the only CAR to significantly secret IL-4, and BAFF-R and CD28 secrete significantly higher amounts of IFNγ.
CAR T Cell Exhaustion

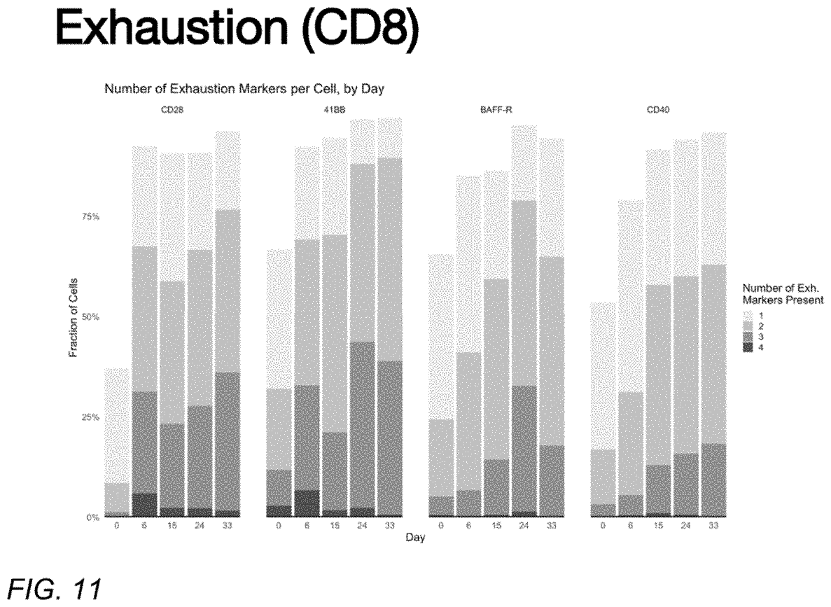

FIG. 11 graphically summarizes the results of experiments performed to illustrate CD8 exhaustion. Number of different exhaustion markers (CD39, LAG3, TIM3, PD1) present per cell at different time points is shown. CD40 and BAFF-R express less of these markers overall, and a lower proportion of the cells have three or more markers present at later time points.

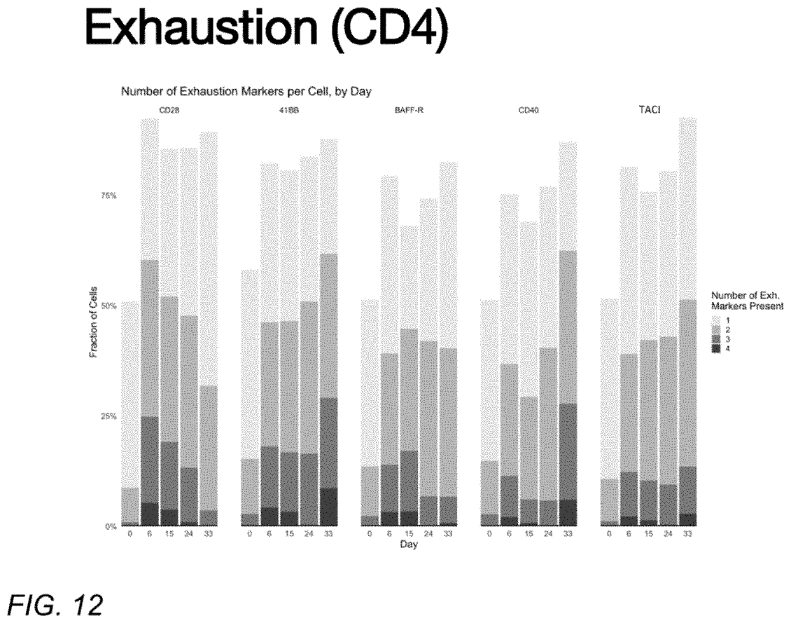

FIG. 12 graphically summarizes the results of experiments performed to illustrate CD4 exhaustion. Number of different exhaustion markers (CD39, LAG3, TIM3, PD1) present per cell at different time points is shown. CD28 and BAFF-R express less of these markers overall, and a lower proportion of the cells have three or more markers present later time points.
Killing

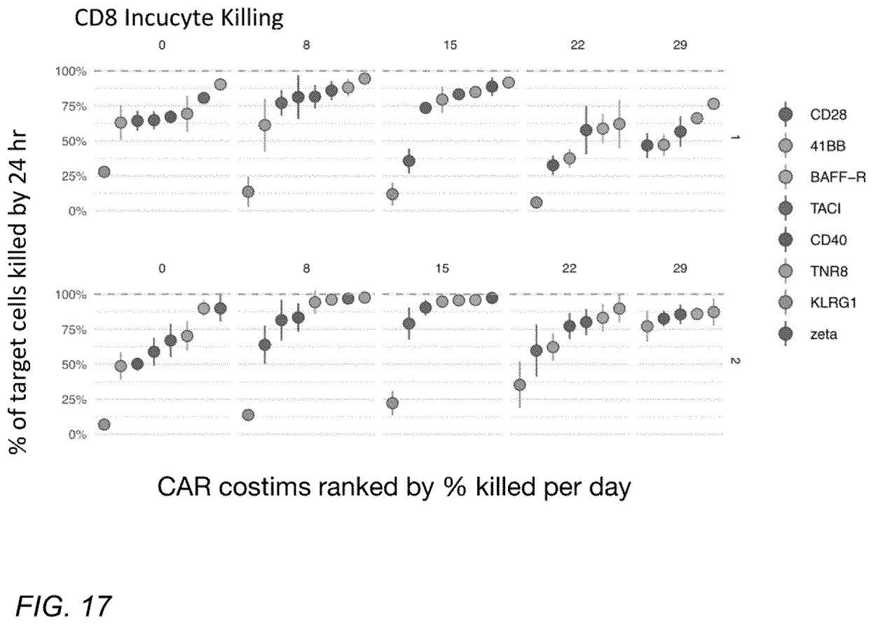

The results of experiments of CD8 Incucyte killing assay are shown in FIG. 17. In these experiments, killing was measured in an live cell time-lapse imaging assay, where mKate-labelled k562 target cells are measured alongside CAR-transduced T cells, which are sorted out of a long-term culture at various times after continuous stimulation with irradiated K562s. Killing is shown as tumor cells remaining in culture after 24 hours compared to K562 cells plated with untransduced T cells

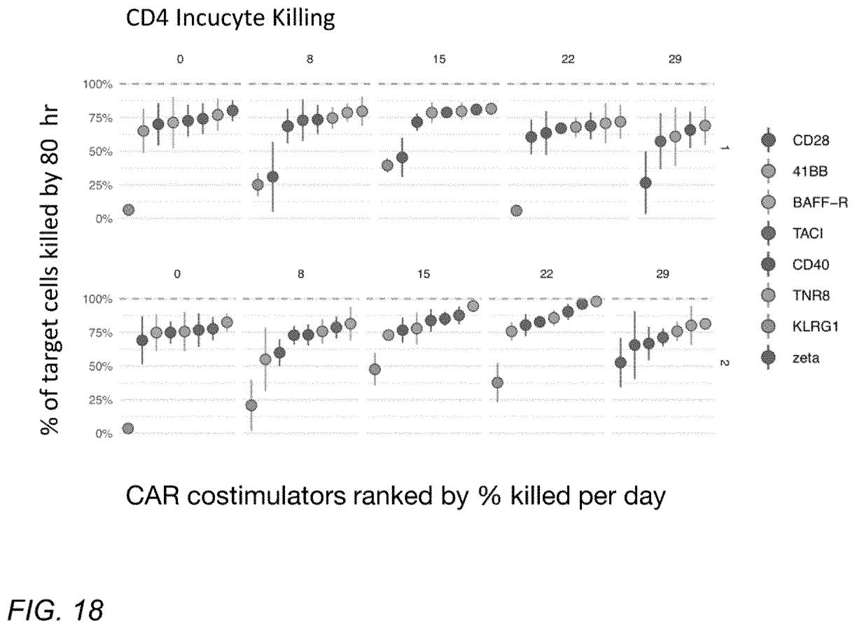

The results of experiments of CD4 Incucyte killing assay are shown in FIG. 18. In these experiments, killing was measured in an live cell time-lapse imaging assay, where mKate-labelled k562 target cells are measured alongside CAR-transduced T cells, which are sorted out of a long-term culture at various times after continuous stimulation with irradiated K562s. Killing is shown as tumor cells remaining in culture after 80 hours compared to K562 cells plated with untransduced T cells.

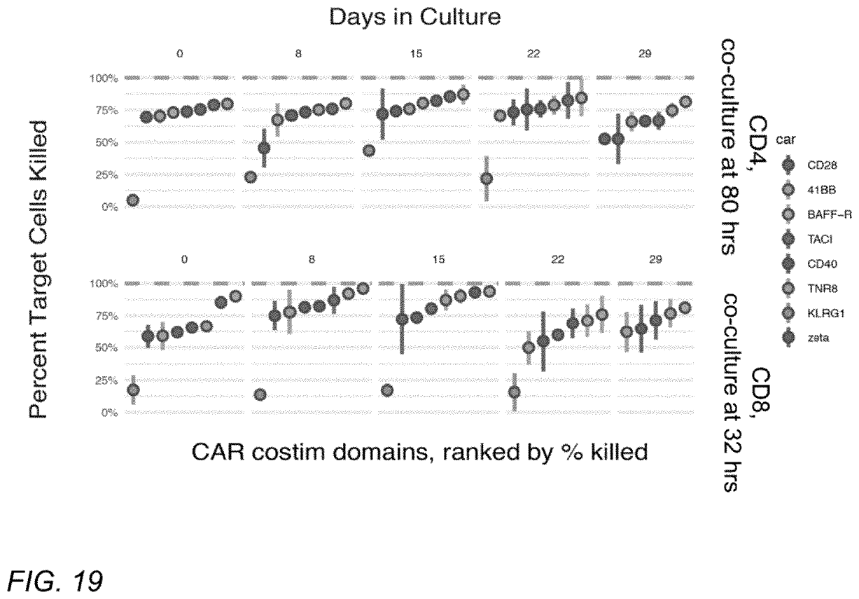

The results of Incucyte killing assays presented in FIGS. 17-18 are summarized in FIG. 19. In these experiments, killing was measured in an live cell time-lapse imaging assay, where mKate-labelled k562 target cells are measured alongside CAR-transduced T cells, which are sorted out of a long-term culture at various times after continuous stimulation with irradiated K562s. Killing is shown as tumor cells remaining in culture after 36/80 (CD4/CD8) hours compared to K562 cells plated with untransduced T cells. Percentage (%) killing is averaged between donors.

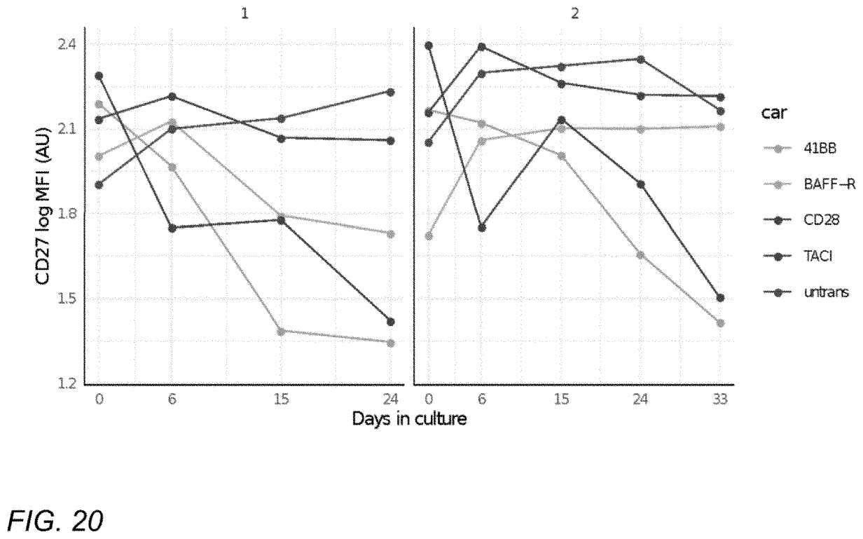

Additional experiments were performed to illustrate that CD27 expression is maintained in BAFF-R and TACI. As shown in FIG. 20, CD27 was stained at various timepoints for each CAR (days 0, 6, 15, 24, and 33 post initial stimulation). To determine the expression level and number of exhaustion markers expressed on each CAR, T cells were stimulated with K562 cells, either with or without CD19 expression, at a 1:1 ratio every three days as described above. For each day CD27 was stained, with the exception of the baseline on Day 0, the cells remained untouched after the last addition of K562 cells 6 days prior to staining. On the 6^{th} day after the last K562 addition, the co-culture was collected by centrifugation at 500×g for 5 minutes, washed twice with flow buffer (PBS+2% FBS), and stained with anti-CD27, -LAG3, -PD1, and -TIM3 antibodies at 4° C. for 30 minutes. The cells were washed twice with flow buffer and analyzed by flow cytometry. CD27 expression is maintained in BAFF-R and TACI CARs similar to untransduced T cells, while CD27 expression is lost in 4-1BB and CD28.

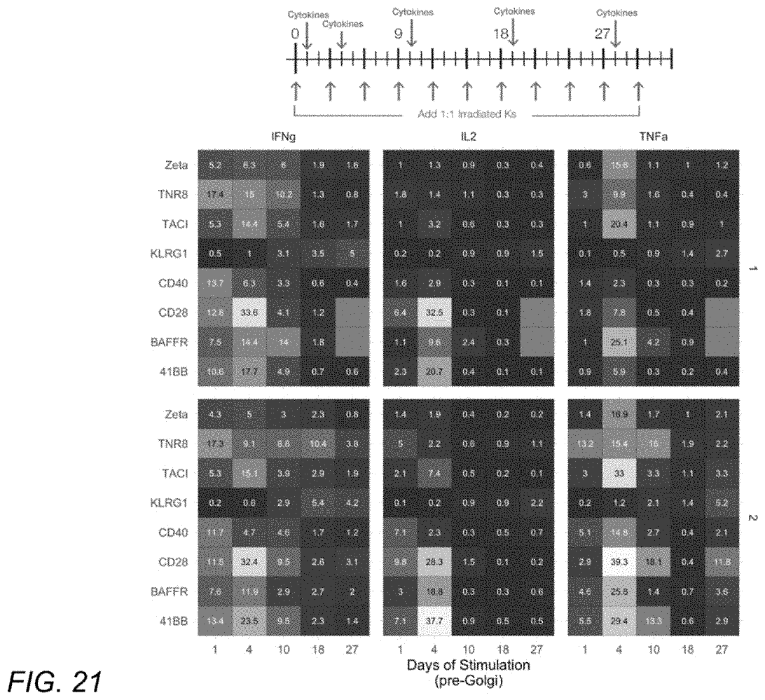

Further experiments were performed to illustrate that CD28, 4-1BB, and Baffr are the highest cytokine producers. As shown in FIG. 21, primary human CD4 T cells were lentivirally transduced with each CAR, sorted for purity, and stimulated with CD19+ or CD19− irradiated K562 tumor cells every three days over a period of 33 days. At 24 hours, 4 days, 10 days, 19 days, and 28 days, the cells were assayed for cytokine production. To determine the degree of cytokine production upon activation of each CAR, 2× Brefeldin A was added 12 hours after the last bolus of K562 cells for an additional 6 hours. Cells were collected by centrifugation at 500×g for 5 minutes, washed twice in flow buffer (PBS+2% FBS), and stained with anti-CD4 or -CD8 antibodies at 4° C. for 20 minutes. The cells were washed twice with flow buffer and 100 μL of fixative (50 μl of flow buffer+50 μL of Invitrogen IC fix) was added to each well. The samples were incubated at room temperature for 1 hour in the dark. After fixation, the cells were spun at 600×g for 5 minutes and resuspended in Cytolast for continued staining the next day. To permeabilize the cells, 200 μL of 1× Permeabilization buffer was added to each well, and cells were immediately spun at 600×g for 5 minutes and stained for intracellular antigens with anti-IL-2, -TNFα, and -IFNy antibodies diluted in permeabilization buffer. The cells were stained in a 50 μL reaction at room temperature for 30 minutes in the dark. The cells were then washed twice with permeabilization buffer and analyzed by flow cytometry. Cd28 was the highest producer of IFNγ and TNFα, 4-1BB was the highest producer of IL-2, and BAFF-R as well as TACI produced similar levels of TNFα as compared to 4-1BB. The number within each box represents the percent of the total T cell population that was producing each cytokine.

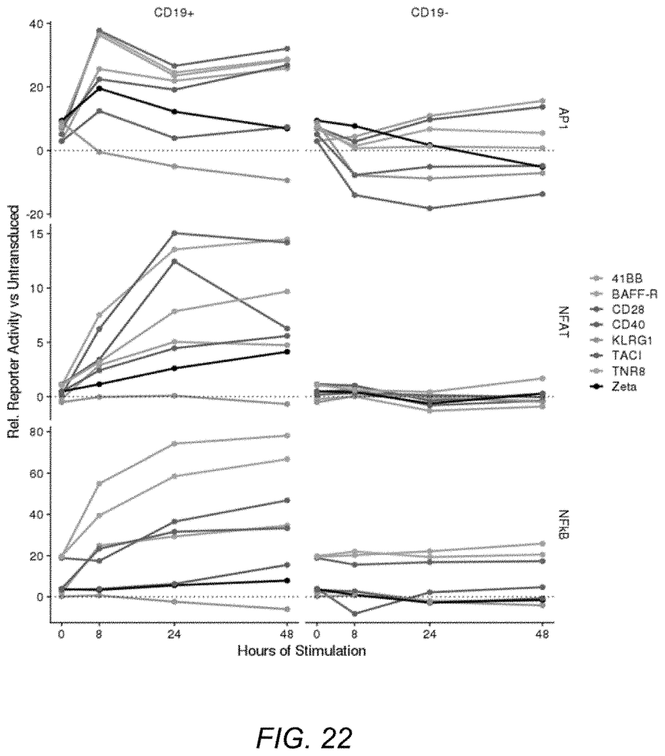

FIG. 22 graphically summarizes the results of experiments where arrayed CARs demonstrate differential signaling dynamics. MCherry-reporter Jurkat cell lines for AP1, NFAT, and NFkB were lentivirally transduced with each CAR, sorted for purity and expression, and stimulated with Cd19+ or CD19− K562 tumor cells. Transcription factor induction was measured via flow cytometry 8, 24, and 48 hours later. Results indicate an antigen specific induction of all three transcription factors with BAFF-R, TACI, and TNR8 showing the highest transcription factor activation.

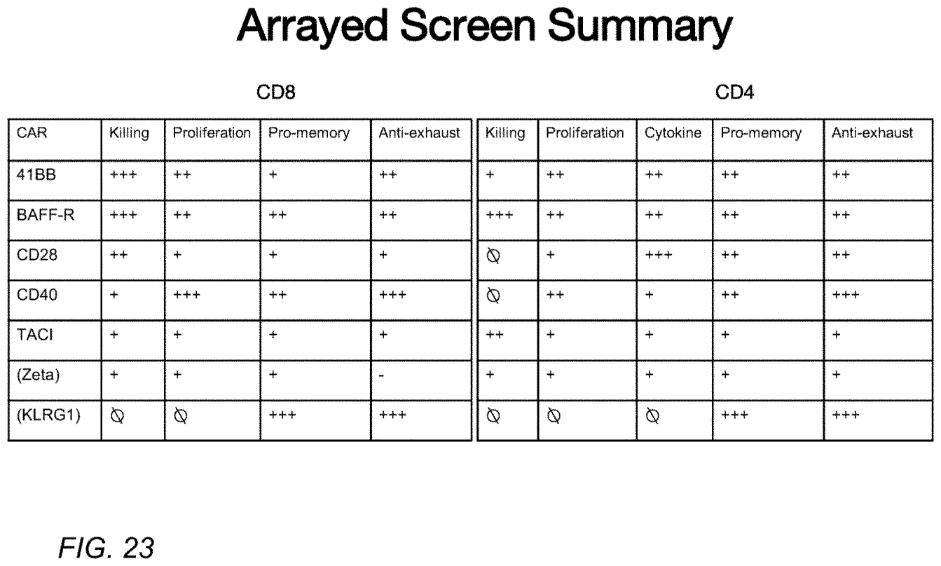

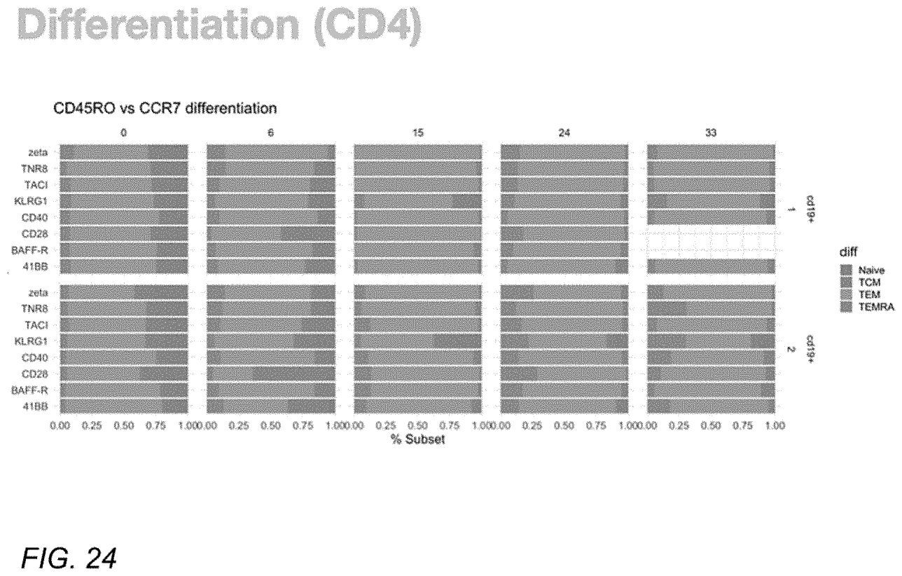

FIG. 23 depicts a qualitative summary of the arrayed screen. Based on the arrayed screen experimental data, to give a broad overview of the properties of each receptor, qualitative ratings were manually assigned to each of the CARs. FIG. 24 schematically summarizes the results of experiments performed to illustrate JCD4 differentiation subsets over time. In these experiments, T cell differentiation subsets are plotted as stacked bar graphs for each day of the arrayed assay. Naive T cells (CD27+/CD45RO−) transition to T Effector cells (TEM) after activation (CD27−/CD45RO+). T central memory cells (TCM) are capable of mounting a long-term immune response upon rechallenge (CD27+/CDRO+). TEMRA (T Effector Memory RA+) cells are less proliferative and have reduced cytokine polyfunctionality, and are the final differentiated state of T cells before apoptosis/death. TACI, 4-1BB and BAFF-R cells have increased TCM and reduced TEMRA populations at later time points, though the effect is variable between donors.

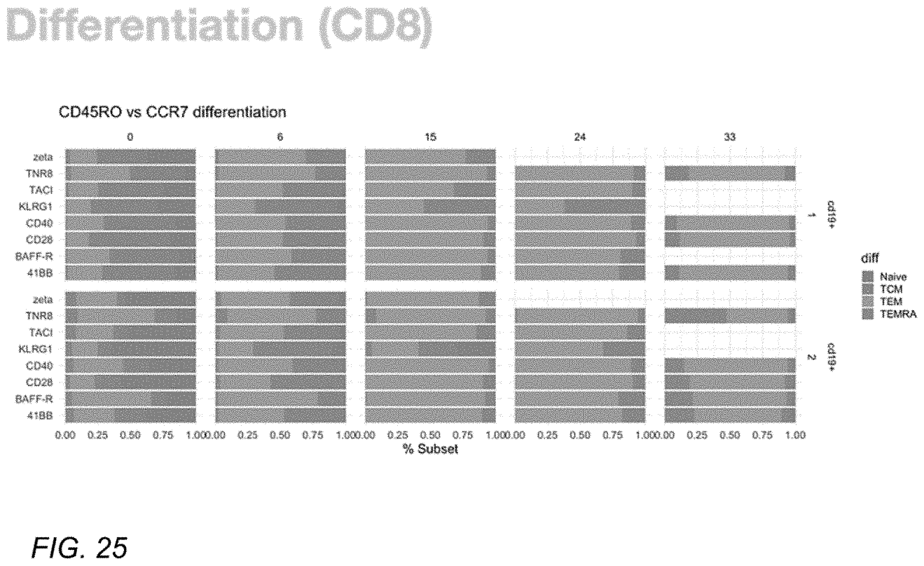

FIG. 25 schematically summarizes the results of experiments performed to illustrate JCD8 differentiation subsets over time. In these experiments, T cell differentiation subsets are plotted as stacked bar graphs for each day of the arrayed assay. Naive T cells (CD27+/CD45RO−) transition to T Effector cells (TEM) after activation (CD27−/CD45RO+). T central memory cells (TCM) are capable of mounting a long-term immune response upon rechallenge (CD27+/CDRO+). TEMRA (T Effector Memory RAP) cells are less proliferative and have reduced cytokine polyfunctionality, and are the final differentiated state of T cells before apoptosis/death. CD40 cells have increased TCM and reduced TEMRA populations at later time points, though the effect is variable between donors.

### Example 3

### CAR-T Proliferation and Activation

IncuCyte Live Cell Analysis

Fibronectin (50 μL, 5 μg/mL) was dispensed to each utilized well of a 96-well plate. Plates were incubated for 60 minutes at room temperature and fibronectin was removed, followed by another 60 minute incubation at room temperature. Both CAR-T cells and live K562 target cells (either expressing mKate and CD19, or only mKate) were spun down and resuspended in Jurkat media+30 U/mL IL-2. Jurkat media (RPMI-1640 medium+10% FBS+1% Pen-Strep+1×GlutaMAX™). This media has less fluorescence than media based on X-VIVO-15. Cells were counted and diluted to 0.25×10⁶/mL each, and 100 μL of each (T cell and Targets) was added to each well for a final assay volume of 200 μL. Each condition was performed in duplicate as long as sufficient cells were available. Plates were allowed to settle at room temperature for 30 minutes before beginning the IncuCyte assay. Images were taken every 60 minutes using the IncuCyte software over the course of the experiments (see relevant figures for total assay times, which varied between conditions).

### Example 4

### BAFF-R and TACI CAR T Cells Eliminate an In Vivo Solid Tumor Model

This Example describes experiments performed to validate the in vitro cytotoxicity assay observation in an in vivo setting by using an established epithelioid mesothelioma solid tumor model (M28).

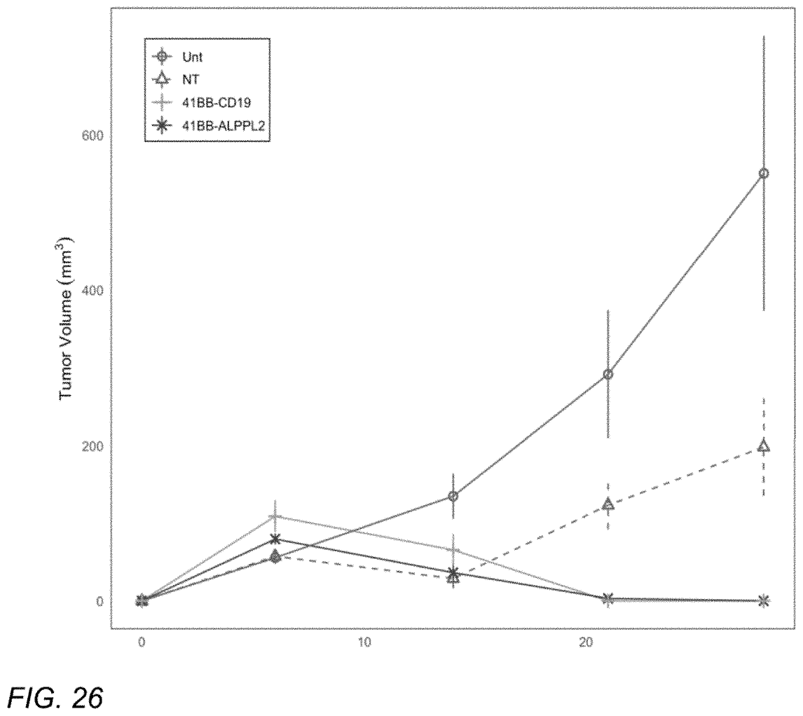

Epithelioid mesothelioma solid tumor model M28 is known to produce durable tumors that require CAR persistence rather than rapid initial proliferation. The slower growth dynamics of the M28 model give a longer window of time to determine clinical efficacy against solid tumors and the propensity for relapse. To utilize the M28 system, CD19 was expressed exogenously on M28 cells, and the cells sorted for CD19 expression similar to the K562s. FIG. 26 summarizes the results from experiments performed to tumor clearance between a CAR against CD19 (which is not normally expressed by M28 tumors and was exogenously expressed), and ALPPL2 (which is a natural tumor antigen on M28). The fact that they show nearly equivalent clearance validates the use of exogenous expression of CD19 on M28s as a measure of CAR performance in this model.

In these experiments, NOD-scid IL2Rgamma^{null} (NSG) mice were injected with 4×10⁶ M28 cells and 7 days later treated them with 6×10⁶ anti-CD19 CART cells. To compare the rejection dynamics and to ensure CARs targeting the exogenously expressed CD19 antigen were comparable to CARs targeting the endogenously expressed ALPPL2 antigen, a side-by-side in vivo experiment were set up utilizing either anti-ALPPL2 4-1BB or anti-CD19 4-1BB CAR treatments. No differences in tumor growth or rejection dynamics were observed between CAR treatments targeting the two antigens (see, e.g., FIG. 26).

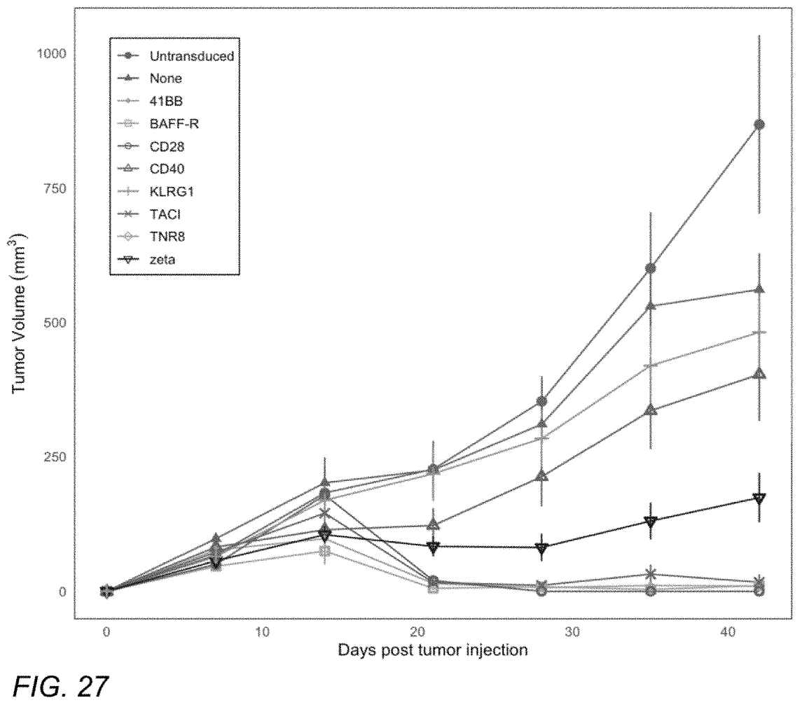

Having confirmed similar tumor rejection between CARs targeting the engineered and natural ligands, the different CAR costimulatory domains were compared head-to-head in the M28 CD19 model. FIG. 27 summarizes the results from experiments performed to validate the in vitro cytotoxicity assay observation in an in vivo setting by using an established epithelioid mesothelioma solid tumor model (M28). In these experiments, tumor samples were (i) left untreated, (ii) treated with untransduced T cells, or (iii) treated with engineered CART cells. Tumor size was monitored over 49 days post tumor injection. CAR T cell variants are compared to untransduced T cells and no T cell controls. No difference was observed between 4-1BB and CD28 CARs at the time points shown herein despite the large amount of evidence from human studies that 4-1BB has increased long-term killing and persistence. Both 4-1BB and CD28, as well as the presently described TACI and BAFF-R CAR treatments, exhibited similar tumor clearance and remission over 50 days (see, e.g., FIG. 27). Additionally, KLRG1 mirrored the results from the in vitro cytotoxicity assay and showed markedly increased tumor burden compared to all CARs, including the CD3-only CAR, while showing modest efficacy compared to untransduced T cells.

### Example 5

### General Experimental Procedures

### Splitting/Plating

Blood samples in the form of leukopaks were obtained from healthy male and female volunteers through STEMCELL Technologies. T cells were isolated via a CD4 or CD8 negative selection kit and frozen. T cells were stimulated 24 hours after thawing with 25 μL of CD3/CD28 beads (Thermo Fisher Dynabeads) per 10⁶ T cells. Concentrated lentivirus was added 48 hours after thawing to reach a transduction rate of under 15% for the pooled library experiments and between 30-50% for the arrayed screens. Virus was removed within 18 hours of addition and cells were expanded. Five days after thawing, the beads were removed via magnetic separation and cells were sorted for GFP expression at least half a log higher than the negative population, and spanning no more than a log in MFI. Cells were plated at 0.5×10⁶ cells/mL and split every three days to this density until 10-14 days after thawing.

For the pooled experiments, stimulation was started on day 10, and day 14 for the arrayed experiments. To stimulate the T cells, they were combined 1:1 with irradiated K562 cells (see next method) that either expressed or did not express surface human CD19, and plated at a density of 0.5×10⁶ T cells/mL. Every three days the cultures were spun, resuspended, and counted to split the T cells and add more irradiated K562 cells. The T cells were counted by adding an aliquot of the resuspended culture to CountBright™ beads and calculating the number of T cells through analysis on a BD X-50 Flow Cytometer. The cultures were restimulated with the addition of additional irradiated K562 cells, 1:1 to total T cells in each culture. They were replated at the density of 0.5×10⁶ T cells/mL. This was repeated for a total of 3-33 days. The media used was X-VIVO 15+5% hAb serum+10 mM NAC neutralized with 1N NaOH+0.5% pen/strep+1× beta-mercaptoethanol.

In Vivo: Spliting, Plating, and T Cell Transfer (ALPPL2 vs. CD19)

Blood samples in the form of leukopaks were obtained from healthy male and female volunteers through STEMCELL Technologies. For in vivo experiments, T cells were isolated via a CD3 negative selection kit and frozen. The T cells were stimulated 24 hours after thawing with 25 μL of CD3/CD28 beads (Thermo Fisher Dynabeads) per 1 ×10⁶ T cells. Concentrated lentivirus was added 48 hours after thawing to reach a transduction rate between 65-90% as assessed by flow cytometry. Virus was removed within 24 hours of addition and the cells were expanded. Five days after thawing, the Dynabeads were removed via magnetic separation and cells were sorted for GFP expression at least half a log higher than the negative population and spanning no more than a log in MFI. The cells were plated at 1×10⁶ cells/mL and split every three days to this density until 10 days after thawing.

Four days prior to tumor injection, M28 CD19+tumor cells were split and 0.75 ×10⁶ cells were plated in one T182 flask for every 2 mice. On day 0, NOD-scid IL2Rgamma^{null} (NSG) mice were injected with 4×10⁶ M28 cells subcutaneously on the right flank and measured the initial tumor growth via caliper 6 days later. On the 10th day after thawing the T cells, and a total of 7 days after M28 cells were injected into the mice, 6×10⁶ GFP positive anti-CD19 CAR T cells were injected via tail vein. Tumors were measured via caliper every 7 days for a total of 30 days.

### In Vivo: Splitting, Plating, and T Cell Transfer (TRAC-KO)

The bulk CD3+ T cells isolated from human PBMCs were stimulated 4 hours after thawing with 25 μL of CD3/CD28 beads (Thermo Fisher Dynabeads) per 1×10⁶ T cells. The Dynabeads were removed via magnetic separation and the cells were replated at 1×10⁶ cells/mL 48 hours after stimulation. After an additional 24 hours, Cas9 and guide RNP were electroporated to knock out the TRAC locus with approximately 98-99% efficiency as assessed via flow cytometry. Immediately after the electroporation, concentrated lentivirus was added to reach a transduction rate between 65-90%. Virus was removed 24 hours after addition and the cells were plated at 1×10⁶ cells/mL and split every three days to this density until 10 days after thawing.

M28 mesothelioma tumor cells were injected subcutaneously into the flanks of NSG mice as stated above. Seven days later, 6×10⁶ GFP+ TRAC-knockout CART cells were transferred intravenously via the tail vein. Tumors were measured via caliper every 7 days for a total of 49 days.

### Irradiation of K562 Cells

Live K562 cells (ATCC® CCL-243TM) were grown up in T182 flasks until confluent. Cells were resuspended to 10⁶/mL on ice and irradiated using a Cesium-137 irradiator for 20 minutes (-200 rad/min) in a 50 mL falcon tube. Cells were then aliquoted and frozen in IMDM media containing 10% DMSO and 10% FBS in liquid nitrogen until needed in the protocol.

### DNA Extraction/Sequencing

After fluorescence activated cell sorting assays, after in vitro growth with target cells, or after transduction as a library abundance baseline measure, T cells containing the CAR library were spun down into pellets, supernatant removed, and frozen at −80° C. Subsequently, genomic DNA from was prepped from cells using either a Machery-Nagel Nucleospin Tissue XS column, a Machery-Nagel Nucleospin column, or a Nucleospin 96 Tissue extraction plate. Manufacturer protocols were followed except for the addition of 10 μg polyadenylated RNA to each sample to increase yield.

After gDNA prep, Picogreen and a plate-based fluorescence reader was used to quantify the extracted genomic DNA. Initial PCR amplification of the costimulatory domain region from the different samples (PCR1) were done in 3 batches with differing numbers of cycles (12, 16 or 22 cycles) depending on the genomic DNA concentration. PCR reactions were performed with Takara ExTAQ to allow for maximum template concentration to be used in the reaction. Reactions were done in 70 μl with between 200 and 1000 ng of DNA used as template depending on the batch as described above.

For the subsequent PCR, to add Illumina barcodes and adapters to the products (PCR2), all products from PCR1 were diluted 15× and 25 μl, of template was used in a 50 μL reaction with Takara ExTAQ. Different forward and reverse primers were used for each sample for PCR2 to add unique custom Illumina IS and 17 barcode sequences to each sample.

Finally, PCR2 products were again quantified using Picogreen in a plate-based fluorescence reader. These products were pooled at 1:1 molar ratio, diluted, and loaded and run on a MiniSeq 2×150 cycle cartridge using the standard manufacturer protocols.

### Sequencing Analysis

After demultiplexing, CAR costimulatory domain sequences in FASTQ format for each sample were aligned using BWA mem. These alignments were then converted into count tables and analyzed using DESeq2 and custom R scripts.

### Differentiation

CCR7 is a receptor found on T cells that induces them to home to secondary lymphoid organs, such as lymph nodes and the spleen. Thus, CCR7 expression can be used as to distinguish between peripheral and central immune cells. CD45RO is expressed by memory T cells. Table 9 shows the percentage of differentiation (CD45RO vs. CCR7) of CD8 T cells having SDs 4-1BB, BAFF-R, CD28, CD40, KLRG1, TACI, TNR8, and null (CD3 only) with CD19 stimulation at days 0, 6, 15, 24, and 33. The T cells are categorized as naïve (CD45RA⁺ CCR7⁺), central memory (T_{CM}, CD45RA⁻ CD45RO⁺ CCR7⁺), effector memory (TEM, CD45RA⁻ CD45RO+CCR7⁻), and effector memory re-expressing CD45RA (TEMRA, CD45RA⁺ CD45RO⁺ CCR7⁻).

The results of experiments performed to illustrate JCD4 differentiation subsets over time are presented in FIG. 24 which shows the percentage of differentiation (CD45RO vs. CCR7) of CD4 T cells having SDs 4-1BB, BAFF-R, CD28, CD40, KLRG1, TACI, TNR8, and null (CD3 only) with CD19 stimulation at days 0, 6, 15, 24, and 33. The T cells are categorized as naïve, T_{CM}, T_{EM}, and T_{EMRA}. In these experiments T cell differentiation subsets are plotted as stacked bar graphs for each day of the arrayed assay. Naive T cells (CD27+/CD45RO−) transition to T Effector cells (TEM) after activation (CD27−/CD45RO+). T central memory cells (TCM) are capable of mounting a long-term immune response upon rechallenge (CD27+/CDRO+). TEMRA (T Effector Memory RA+) cells are less proliferative and have reduced cytokine polyfunctionality, and are the final differentiated state of T cells before apoptosis/death. TACI, 4-1BB and BAFF-R cells have increased TCM and reduced TEMRA populations at later time points, though the effect is variable between donors.

The results of experiments performed to evaluate JCD4 differentiation subsets over time are presented in FIG. 24 which shows the percentage of differentiation (CD45RO vs. CCR7) of CD4 T cells having SDs 4-1BB, BAFF-R, CD28, CD40, KLRG1, TACI, TNR8, and null (CD3 only) with CD19 stimulation at days 0, 6, 15, 24, and 33. The T cells are categorized as naïve, T_{CM}, T_{EM}, and T_{EMRA}. In these experiments T cell differentiation subsets are plotted as stacked bar graphs for each day of the arrayed assay. Naive T cells (CD27+/CD45RO−) transition to T Effector cells (TEM) after activation (CD27−/CD45RO+). T central memory cells (TCM) are capable of mounting a long-term immune response upon rechallenge (CD27+/CDRO+). TEMRA (T Effector Memory RA+) cells are less proliferative and have reduced cytokine polyfunctionality, and are the final differentiated state of T cells before apoptosis/death. As shown in FIG. 24, TACI, 4-1BB and BAFF-R cells have increased TCM and reduced TEMRA populations at later time points, though the effect is variable between donors.

The results of experiments performed to evaluate JCD4 differentiation subsets over time are presented in FIG. 25. In these experiments, T cell differentiation subsets are plotted as stacked bar graphs for each day of the arrayed assay. Naive T cells (CD27+/CD45RO−) transition to T Effector cells (TEM) after activation (CD27−/CD45RO+). T central memory cells (TCM) are capable of mounting a long-term immune response upon rechallenge (CD27+/CDRO+). TEMRA (T Effector Memory RA+) cells are less proliferative and have reduced cytokine polyfunctionality, and are the final differentiated state of T cells before apoptosis/death. As shown in FIG. 25, CD40 cells have increased TCM and reduced TEMRA populations at later time points, though the effect is variable between donors.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A chimeric receptor comprising:
a) an extracellular antigen-binding domain capable of binding to a target antigen;
b) a transmembrane domain; and
c) an intracellular signal transduction domain comprising an intracellular signaling domain (SD) derived from a signaling molecule selected from the group consisting of 4-1BB, BAFF-R, BCMA, BTLA, CD2, CD200R, CD244, CD28, CD300a, CD300f, CD40, CD7, CD72, CD96, CRACC, CRTAM, CTLA4, CXADR, DC-SIGN, GITR, HAVCR2, ICOS, ILT2, ILT3, ILT4, KIR2DL1, KIR3DL1, KLRG1, LAG3, LAIR1, NKG2D, NKR-P1A, NTB-A, PD1, Siglec-3, TACI, TIGIT, TLT-1, and TNR8 (CD30).

2. The receptor of claim 1, wherein the intracellular signal transduction domain comprises a modulatory SD capable of mediating a co-stimulatory signal derived from a signaling molecule selected from the group consisting of BAFF-R, CD40, TACI, CD2, CD7, TNR8 (CD30), and NTB-A.

3. The receptor of claim 2, wherein the modulatory SD is derived from:
a) BAFF-R and comprises the amino acid sequence of SEQ ID NO: 24 or a variant thereof having at least about 80% sequence identity to SEQ ID NO: 24;
b) CD40 and comprises the amino acid sequence of SEQ ID NO: 10 or a variant thereof having at least about 80% sequence identity to SEQ ID NO: 10;
c) TACI and comprises the amino acid sequence of SEQ ID NO: 25 or a variant thereof having at least about 80% sequence identity to SEQ ID NO: 25;
d) CD2 and comprises the amino acid sequence of SEQ ID NO: 19 or a variant thereof having at least about 80% sequence identity to SEQ ID NO: 19;
e) CD7 and comprises the amino acid sequence of SEQ ID NO: 3 or a variant thereof having at least about 80% sequence identity to SEQ ID NO: 3;
f) CD30 and comprises the amino acid sequence of SEQ ID NO: 21 or a variant thereof having at least about 80% sequence identity to SEQ ID NO: 21; or
g) NTB-A and comprises the amino acid sequence of SEQ ID NO: 29 or a variant thereof having at least about 80% sequence identity to SEQ ID NO: 29.

4. The chimeric receptor of claim 2 or 3, wherein the intracellular signal transduction domain further comprises an activation domain.

5. The chimeric receptor of claim 4, wherein the activation domain comprises one or more immunoreceptor tyrosine-based activation motifs (ITAMs).

6. The chimeric receptor of claim 5, wherein the activation domain is 80, 85, 90, 95, 96, 97, 98, 99, or 100% identical to a CD3 activation domain.

7. The chimeric receptor of any one of claims 2 to 6, wherein the extracellular antigen-binding domain comprises an antibody moiety capable of binding to the target antigen.

8. The chimeric receptor of claim 7, wherein the antibody moiety is a scFv.

9. The chimeric receptor of any one of claims 2 to 8, wherein the target antigen is CD19, CD20, or MAGE.

10. The chimeric receptor of any one of claims 2 to 6, wherein the extracellular antigen-binding domain comprises an extracellular domain derived from an inhibitory immune checkpoint molecule or a binding moiety capable of binding to a ligand of the inhibitory immune checkpoint molecule.

11. The chimeric receptor of claim 1, wherein the intracellular signal transduction domain comprises a modulatory SD capable of mediating an inhibitory signal derived from a signaling molecule selected from the group consisting of KLRG1, DC-SIGN, NKG2D, and NKR-P1A.

12. The chimeric receptor of claim 11, wherein the modulatory SD is derived from:
a) KLRG1 and comprises the amino acid sequence of SEQ ID NO: 32 or a variant thereof having at least about 80% sequence identity to SEQ ID NO: 32;
b) DC-SIGN and comprises the amino acid sequence of SEQ ID NO: 43 or a variant thereof having at least about 80% sequence identity to SEQ ID NO: 43;
c) NKG2D and comprises the amino acid sequence of SEQ ID NO: 22 or a variant thereof having at least about 80% sequence identity to SEQ ID NO: 22; or
d) NKR-P1A and comprises the amino acid sequence of SEQ ID NO: 33 or a variant thereof having at least about 80% sequence identity to SEQ ID NO: 33.

13. The chimeric receptor of claim 11 or 12, wherein the extracellular antigen-binding domain comprises an extracellular domain derived from a stimulatory immune checkpoint molecule or a binding moiety capable of binding to a ligand of the stimulatory immune checkpoint molecule.

14. A recombinant nucleic acid comprising a nucleotide sequence encoding a chimeric receptor according to any one of claims 1 to 13.

15. The recombinant nucleic acid of claim 14, wherein the nucleotide sequence is incorporated into an expression cassette or an expression vector.

16. The recombinant nucleic acid of claim 15, wherein the expression vector is a viral vector.

17. The recombinant nucleic acid of claim 16, wherein the viral vector is a lentiviral vector, an adenovirus vector, an adeno-associated virus vector, or a retroviral vector.

18. A composition comprising a recombinant nucleic acid according to any one of claims 14 to 17, wherein the composition is formulated for introducing the recombinant nucleic acid into a cell.

19. The composition of claim 18, wherein the composition is formulated as a lipid nanoparticle (LNP), liposome, or viral particle.

20. A recombinant immune cell comprising:
a) a chimeric receptor according to any one of claims 1 to 13; or
b) a recombinant nucleic acid according any one of claims 14 to 17.

21. The recombinant immune cell of claim 20, wherein the recombinant immune cell is a recombinant T cell.

22. The recombinant T cell of claim 21, wherein the recombinant T cell is a recombinant CD4⁺ T cell or a recombinant CD8⁺ T cell.

23. The recombinant immune cell of any one of claims 20 to 22, wherein the recombinant immune cell comprises a chimeric receptor according to any one of claims 2 to 10 and has one or more of the following properties:
a) enhanced proliferation in response to stimulation with the target antigen as compared to a corresponding cell that comprises a corresponding receptor where the intracellular SD is derived from CD28 or 4-1BB;
b) enhanced expression of activation marker CD69 in response to stimulation with the target antigen as compared to a corresponding cell that comprises a corresponding receptor where the intracellular SD is derived from CD28 or 4-1BB;
c) enhanced expression of IFNγ, TNFα, IL-2, and/or IL-4 in response to stimulation with the target antigen as compared to a corresponding cell that comprises a corresponding receptor where the intracellular SD is derived from CD28 or 4-1BB;
d) enhanced resistance to exhaustion in response to stimulation with the target antigen as compared to a corresponding cell that comprises a corresponding receptor where the intracellular SD is derived from CD28 or 4-1BB; and
e) enhanced killing of target cells expressing the target antigen as compared to a corresponding cell that comprises a corresponding receptor where the intracellular SD is derived from CD28 or 4-1BB.

24. The recombinant immune cell of any one of claims 20 to 22, wherein the recombinant immune cell comprises a chimeric receptor according to any one of claims 11 to 13 and has one or more of the following properties:
a) reduced proliferation in response to stimulation with the target antigen as compared to a corresponding cell that comprises a corresponding receptor where the intracellular SD is derived from an inhibitory signaling receptor selected from CTLA-4 and PD1;
b) reduced expression of activation marker CD69 in response to stimulation with the target antigen as compared to a corresponding cell that comprises a corresponding receptor where the intracellular SD is derived from an inhibitory signaling receptor selected from CTLA-4 and PD1
c) reduced expression of IFNγ, TNFα, and/or IL-2 in response to stimulation with the target antigen as compared to a corresponding cell that comprises a corresponding receptor where the intracellular SD is derived from an inhibitory signaling receptor selected from CTLA-4 and PD1;
d) reduced resistance to exhaustion in response to stimulation with the target antigen as compared to a corresponding cell that comprises a corresponding receptor where the intracellular SD is derived from an inhibitory signaling receptor selected from CTLA-4 and PD1; and
e) reduced killing of target cells expressing the target antigen as compared to a corresponding cell that comprises a corresponding receptor where the intracellular SD is derived from an inhibitory signaling receptor selected from CTLA-4 and PD1.

25. A pharmaceutical composition comprising a pharmaceutically acceptable carrier and one or more of the following:
a) a recombinant nucleic acid according to any one of claims 14 to 17; and
b) a recombinant immune cell according to any one of claims 20 to 24.

26. The pharmaceutical composition of claim 25, wherein the pharmaceutical composition comprises a recombinant nucleic acid according to any one of claims 14 to 17.

27. The pharmaceutical composition of claim 26, wherein the recombinant nucleic acid is encapsulated in an LNP, liposome, or viral particle.

28. A method for modulating the activity of an immune cell, comprising:
a) expressing a chimeric receptor according to any one of claims 1 to 13 in the immune cell; and
b) contacting the immune cell with the target antigen.

29. The method of claim 28, wherein the contacting is carried out in vivo, ex vivo, or in vitro.

30. The method of claim 28 or 29, wherein the immune cell comprises a chimeric receptor according to any one of claims 2 to 10, and the activity of the immune cell is increased following the contacting as compared to the immune cell prior to the contacting or a corresponding immune cell that does not express the chimeric receptor.

31. The method of claim 28 or 29, wherein the immune cell comprises a chimeric receptor according to any one of claims 11 to 13, and the activity of the immune cell is decreased following the contacting as compared to the immune cell prior to the contacting or a corresponding immune cell that does not express the chimeric receptor.

32. A method for modulating an immune response to a first antigen in an individual, comprising administering to the individual an effective amount of recombinant immune cells according to any one of claims 20 to 22.

33. The method of claim 32, wherein the recombinant immune cells comprise a chimeric receptor according to any one of claims 2 to 10, the chimeric receptor is capable of binding to the first antigen, and an immune response to the first antigen is stimulated.

34. The method of claim 32, wherein the recombinant immune cells comprise a chimeric receptor according to any one of claims 11 to 13, the chimeric receptor is capable of binding to the first antigen, and an immune response to the first antigen is inhibited.

35. The method of claim 32, wherein the recombinant immune cells comprise
a) a chimeric receptor according to any one of claims 11 to 13, wherein the chimeric receptor is activated by binding to a repressor antigen; and
b) a second receptor capable of binding to the first antigen to stimulate an immune response to the first antigen.

36. The method of claim 35, wherein the first antigen is present on a target cell to which an immune response is desired and the repressor antigen is present on a non-target cell to which an immune response is not desired.

37. The method of claim 35, further comprising administering to the individual an effective amount of the repressor antigen such that an immune response to the first antigen is inhibited.

38. A method for modulating an immune response to an antigen in an individual, comprising administering to the individual an effective amount of a pharmaceutical composition according to claim 26 or 27 such that the recombinant nucleic acid is introduced into immune cells in the individual capable of mediating the immune response.

39. The method of claim 38, wherein the pharmaceutical composition comprises a chimeric receptor according to any one of claims 2 to 10, the chimeric receptor is capable of binding to the antigen, and an immune response to the antigen is stimulated.

40. The method of claim 38, wherein the pharmaceutical composition comprises a chimeric receptor according to any one of claims 11 to 13, the chimeric receptor is capable of binding to the antigen, and an immune response to the antigen is inhibited.

41. A method for treating health condition in an individual in need thereof, comprising administering to the individual an effective amount of recombinant immune cells according to any one of claims 20 to 22, wherein the recombinant immune cells treat the health condition.

42. The method of claim 41, wherein the recombinant immune cells comprise a chimeric receptor according to any one of claims 2 to 10, and the health condition is characterized by a pathogenic cell expressing the target antigen of the chimeric receptor.

43. The method of claim 41, wherein the recombinant immune cells comprise
a) a chimeric receptor according to any one of claims 11 to 13, wherein the chimeric receptor is activated by binding to a repressor antigen; and
b) a second receptor capable of binding to a second antigen associated with the health condition to stimulate an immune response to the second antigen.

44. The method of claim 43, wherein the second antigen is present on a pathogenic cell associated with the health condition and the repressor antigen is present on a non-pathogenic cell.

45. The method of claim 43, wherein the method further comprises administering to the individual an effective amount of the repressor antigen such that an adverse effect in the individual mediated by the recombinant immune cells is inhibited.

46. A method for treating a health condition in an individual in need thereof, comprising administering to the individual an effective amount of the pharmaceutical composition of claim 26 or 27 such that the recombinant nucleic acid is introduced into immune cells in the individual to generate recombinant immune cells that treat the health condition.

47. The method of claim 46, wherein the pharmaceutical composition comprises a chimeric receptor according to any one of claims 2 to 10, and the health condition is characterized by a pathogenic cell expressing the target antigen.

48. The method of claim 46, wherein the pharmaceutical composition comprises a chimeric receptor according to any one of claims 11 to 13, and the health condition is characterized by an adverse immune response to the target antigen.

49. A kit for modulating an activity of an immune cell, modulating an immune response in an individual, or treating a health condition in an individual in need thereof, wherein the kit comprises one or more of the following:
a) a chimeric receptor according to any one of claims 1 to 13;
b) a recombinant nucleic acid according to any one of claims 14 to 17;
c) a recombinant immune cell according to any one of claims 20 to 24; and
d) a pharmaceutical composition according to any one of claims 25 to 27, and instructions for use thereof

50. A kit comprising a plurality of the chimeric receptors according to any one of claims 1 to 13, wherein the plurality of chimeric receptors collectively comprises at least 3, 4, 5, 6, 7, 8, 9, 10, 15, 20, 25, 30, 35, 40, 45, 48, or 50 different SDs.

51. The kit of claim 50, further comprising a chimeric receptor according to claim 1 wherein the intracellular SD is replaced with an intracellular SD derived from CD28 and/or 4-1BB.

52. A kit comprising a plurality of the recombinant nucleic acids according to any one of claims 14 to 17, wherein the plurality of recombinant nucleic acids encode a plurality of chimeric receptors collectively comprising at least 3, 4, 5, 6, 7, 8, 9, 10, 15, 20, 25, 30, 35, 40, 45, 48, or 50 different SDs.

53. A kit comprising a plurality of the recombinant immune cells according to any one of claims 20 to 24, wherein the plurality of recombinant immune cells comprise a plurality of chimeric receptors collectively comprising at least 3, 4, 5, 6, 7, 8, 9, 10, 15, 20, 25, 30, 35, 40, 45, 48, or 50 different SDs.

54. The chimeric receptor of any one of claims 1 to 13, the recombinant nucleic acid of any one of claims 14 to 17, the recombinant immune cell of any one of claims 20 to 24, or the pharmaceutical composition of any one of claims 25 to 27 for use in the treatment of a health condition.

55. The chimeric receptor of any one of claims 1 to 13, the recombinant nucleic acid of any one of claims 14 to 17, the recombinant immune cell of any one of claims 20 to 24, or the pharmaceutical composition of any one of claims 25 to 27 for use in the manufacture of a medicament.


SEQUENCE LISTING (121 sequences)

SEQ ID NO: 1 - protein, 35 aa, Artificial sequence
  Synthetic construct
  MISC_FEATURE: ICOS
TKKKYSSSVHDPNGEYMFMRAVNTAKKSRLTDVTL

SEQ ID NO: 2 - protein, 39 aa, Artificial sequence
  Synthetic construct
  MISC_FEATURE: CTLA4
SLSKMLKKRSPLTTGVYVKMPPTEPECEKQFQPYFIPIN

SEQ ID NO: 3 - protein, 40 aa, Artificial sequence
  Synthetic construct
  MISC_FEATURE: CD7
ARTQIKKLCSWRDKNSAACVVYEDMSHSRCNTLSSPNQYQ

SEQ ID NO: 4 - protein, 42 aa, Artificial sequence
  Synthetic construct
  MISC_FEATURE: 4-1BB
KRGRKKLLYIFKQPFMRPVQTTQEEDGCSCRFPEEEEGGCEL

SEQ ID NO: 5 - protein, 43 aa, Artificial sequence
  Synthetic construct
  MISC_FEATURE: CD28
WVRSKRSRLLHSDYMNMTPRRPGPTRKHYQPYAPPRDFAAYRS

SEQ ID NO: 6 - protein, 49 aa, Artificial sequence
  Synthetic construct
  MISC_FEATURE: CD27
HQRRKYRSNKGESPVEPAEPCHYSCPREEEGSTIPIQEDYRKPEPACSP

SEQ ID NO: 7 - protein, 51 aa, Artificial sequence
  Synthetic construct
  MISC_FEATURE: LAG3
RRQWRPRRFSALEQGIHPPQAQSKIEELEQEPEPEPEPEPEPEPEPEPEQL

SEQ ID NO: 8 - protein, 54 aa, Artificial sequence
  Synthetic construct
  MISC_FEATURE: GITR
HIWQLRSQCMWPRETQLLLEVPPSTEDARSCQFPEEERGERSAEEKGRLGDLWV

SEQ ID NO: 9 - protein, 61 aa, Artificial sequence
  Synthetic construct
  MISC_FEATURE: CD226
NRRRRRERRDLFTESWDTQKAPNNYRSPISTSQPTNQSMDDTREDIYVNYPTFSRRPKTRV

SEQ ID NO: 10 - protein, 62 aa, Artificial sequence
  Synthetic construct
  MISC_FEATURE: CD40
KKVAKKPTNKAPHPKQEPQEINFPDDLPGSNTAAPVQETLHGCQPVTQEDGKESRISVQERQ

SEQ ID NO: 11 - protein, 77 aa, Artificial sequence
  Synthetic construct
  MISC_FEATURE: TIM-3
KWYSHSKEKIQNLSLISLANLPPSGLANAVAEGIRSEENIYTIEENVYEVEEPNEYYCYVSSRQQPSQPLGCRFAMP

SEQ ID NO: 12 - protein, 80 aa, Artificial sequence
  Synthetic construct
  MISC_FEATURE: TIGIT
RKKKALRIHSVEGDLRRKSAGQEEWSPSAPSPPGSCVQAEAAPAGLCGEQRGEDCAELHDYFNVLSYRSLGNCSFFTETG

SEQ ID NO: 13 - protein, 83 aa, Artificial sequence
  Synthetic construct
  MISC_FEATURE: CRTAM
KLRKAHVIWKKENEVSEHTLESYRSRSNNEETSSEEKNGQSSHPMRCMNYITKLYSEAKTKRKENVQHSKLEEKHIQVPESIV

SEQ ID NO: 14 - protein, 98 aa, Artificial sequence
  Synthetic construct
  MISC_FEATURE: PD-1
ICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 15 - protein, 98 aa, Artificial sequence
  Synthetic construct
  MISC_FEATURE: TNFRSF25
TYRHCWPHKPLVTADEAGMEALTPPPATHLSPLDSAHTLLAPPDSSEKICTVQLVGNSWTPGYPETQEALCPQVTWSWDQLPSRALGPAAAPTLSPES

SEQ ID NO: 16 - protein, 101 aa, Artificial sequence
  Synthetic construct
  MISC_FEATURE: LAIR1
HRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 17 - protein, 107 aa, Artificial sequence
  Synthetic construct
  MISC_FEATURE: CXAR
CCRKKRREEKYEKEVHHDIREDVPPPKSRTSTARSYIGSNHSSLGSMSPSNMEGYSKTQYNQVPSEDFERTPQSPTLPPAKVAAPNLSRMGAIPVMIPAQSKDGSIV

SEQ ID NO: 18 - protein, 114 aa, Artificial sequence
  Synthetic construct
  MISC_FEATURE: BTLA
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 19 - protein, 117 aa, Artificial sequence
  Synthetic construct
  MISC_FEATURE: CD2
TKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 20 - protein, 120 aa, Artificial sequence
  Synthetic construct
  MISC_FEATURE: 2B4
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 21 - protein, 187 aa, Artificial sequence
  Synthetic construct
  MISC_FEATURE: CD30
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 22 - protein, 51 aa, Artificial sequence
  Synthetic construct
  MISC_FEATURE: NKG2D
MGWIRGRRSRHSWEMSEFHNYNLDLKKSDFSTRWQKQRCPVVKSKCRENAS

SEQ ID NO: 23 - protein, 48 aa, Artificial sequence
  Synthetic construct
  MISC_FEATURE: TIM-1, aa
  MISC_FEATURE: TIM-1
KKYFFKKEVQQLSVSFSSLQIKALQNAVEKEVQAEDNIYIENSLYATD

SEQ ID NO: 24 - protein, 85 aa, Artificial sequence
  Synthetic construct
  MISC_FEATURE: BAFF-R
SWRRRQRRLRGASSAEAPDGDKDAPEPLDKVIILSPGISDATAPAWPPPGEDPGTTPPGHSVPVPATELGSTELVTTKTAGPEQQ

SEQ ID NO: 25 - protein, 111 aa, Artificial sequence
  Synthetic construct
  MISC_FEATURE: TACI
ACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 26 - protein, 107 aa, Artificial sequence
  Synthetic construct
  MISC_FEATURE: BCMAI
RKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 27 - protein, 95 aa, Artificial sequence
  Synthetic construct
  MISC_FEATURE: CD72
MAEAITYADLRFVKAPLKKSISSRLGQDPGADDDGEITYENVQVPAVLGVPSSLASSVLGDKAAVKSEQPTASWRAVTSPAVGRILPCRTTCLRY

SEQ ID NO: 28 - protein, 45 aa, Artificial sequence
  Synthetic construct
  MISC_FEATURE: CD96
RKWCQYQKEIMERPPPFKPPPPPIKYTCIQEPNESDLPYHEMETL

SEQ ID NO: 29 - protein, 84 aa, Artificial sequence
  Synthetic construct
  MISC_FEATURE: NTB-A
LRKRRDSLSLSTQRTQGPAESARNLEYVSVSPTNNTVYASVTHSNRETEIWTPRENDTITIYSTINHSKESKPTFSRATALDNV

SEQ ID NO: 30 - protein, 88 aa, Artificial sequence
  Synthetic construct
  MISC_FEATURE: CRACC
WFLKRERQEEYIEEKKRVDICRETPNICPHSGENTEYDTIPHTNRTILKEDPANTVYSTVEIPKKMENPHSLLTMPDTPRLFAYENVI

SEQ ID NO: 31 - protein, 82 aa, Artificial sequence
  Synthetic construct
  MISC_FEATURE: Siglec-3.9.9
KTHRRKAARTAVGRNDTHPTTGSASPKHQKKSKLHGPTETSSCSGAAPTVEMDEELHYASLNFHGMNPSKDTSTEYSEVRTQ

SEQ ID NO: 32 - protein, 38 aa, Artificial sequence
  Synthetic construct
  MISC_FEATURE: KLRG1
MTDSVIYSMLELPTATQAQNDYGPQQKSSSSRPSCSCL

SEQ ID NO: 33 - protein, 45 aa, Artificial sequence
  Synthetic construct
  MISC_FEATURE: NKR-P1A
MDQQAIYAELNLPTDSGPESSSPSSLPRDVCQGSPWHQFALKLSC

SEQ ID NO: 34 - protein, 168 aa, Artificial sequence
  Synthetic construct
  MISC_FEATURE: ILT2
LRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 35 - protein, 83 aa, Artificial sequence
  Synthetic construct
  MISC_FEATURE: KIR2DL1
RWCSNKKNAAVMDQESAGNRTANSEDSDEQDPQEVTYTQLNHCVFTQRKITRPSQRPKTPPTDIIVYTELPNAESRSKVVSCP

SEQ ID NO: 36 - protein, 84 aa, Artificial sequence
  Synthetic construct
  MISC_FEATURE: KIR3DL1
HLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 37 - protein, 168 aa, Artificial sequence
  Synthetic construct
  MISC_FEATURE: ILT3
QHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 38 - protein, 116 aa, Artificial sequence
  Synthetic construct
  MISC_FEATURE: ILT4
LRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 39 - protein, 128 aa, Artificial sequence
  Synthetic construct
  MISC_FEATURE: TLT-1
MAKRKQGNRLGVCGRFLSSRVSGMNPSSVVHHVSDSGPAAELPLDVPHIRLDSPPSFDNTTYTSLPLDSPSGKPSLPAPSSLPPLPPKVLVCSKPVTYATVIFPGGNKGGGTSCGPAQNPPNNQTPSS

SEQ ID NO: 40 - protein, 61 aa, Artificial sequence
  Synthetic construct
  MISC_FEATURE: CD200R
KVNGCRKYKLNKTESTPVVEEDEMQPYASYTEKNNPLYDTTNKVKASEALQSEVDTDLHTL

SEQ ID NO: 41 - protein, 98 aa, Artificial sequence
  Synthetic construct
  MISC_FEATURE: CD300a
RMFQKWIKAGDHSELSQNPKQAATQSELHYANLELLMWPLQEKPAPPREVEVEYSTVASPREELHYASVVFDSNTNRIAAQRPREEEPDSDYSVIRKT

SEQ ID NO: 42 - protein, 113 aa, Artificial sequence
  Synthetic construct
  MISC_FEATURE: DC-SIGN
WRMMKYQQKAAGMSPEQVLQPLEGDLCYADLTLQLAGTSPQKATTKLSSAQVDQVEVEYVTMASLPKEDISYASLTLGAEDQEPTYCNMGHLSSHLPGRGPEEPTEYSTISRP

SEQ ID NO: 43 - protein, 37 aa, Artificial sequence
  Synthetic construct
  MISC_FEATURE: Allergin-1
MSDSKEPRLQQLGLLEEEQLRGLGFRQTRGYKSLAGC

SEQ ID NO: 44 - protein, 95 aa, Artificial sequence
  Synthetic construct
  MISC_FEATURE: PIR-B
LPKYKTRKAMRNNVPRDRGDTAMEVGIYANILEKQAKEESVPEVGSRPCVSTAQDEAKHSQELQYATPVFQEVAPREQEACDSYKSGYVYSELNF

SEQ ID NO: 45 - protein, 178 aa, Artificial sequence
  Synthetic construct
  MISC_FEATURE: CD28.wt
RRRHRGKFRKDVQKEKDLQLSSGAEEPITRKGELQKRPNPAAATQEESLYASVEDMQTEDGVELNSWTPPEEDPQGETYAQVKPSRLRKAGHVSPSVMSREQLNTEYEQAEEGQGANNQAAESGESQDVTYAQLCSRTLRQGAAASPLSQAGEAPEEPSVYATLAAARPEAVPKDMEQ

SEQ ID NO: 46 - protein, 42 aa, Artificial sequence
  Synthetic construct
  MISC_FEATURE: ICOS.wt
VRSKRSRLLHSDYMNMTPRRPGPTRKHYQPYAPPRDFAAYRS

SEQ ID NO: 47 - protein, 39 aa, Artificial sequence
  Synthetic construct
  MISC_FEATURE: ICOS
CCWLTKKKYSSSVHDPNGEYMFMRAVNTAKKSRLTDVTL

SEQ ID NO: 48 - DNA, 105 bp, Artificial sequence
  Synthetic construct
  misc_feature: CTLA4
accaagaaaaagtacagcagcagcgtgcacgaccccaacggcgagtacatgttcatgcgggccgtgaacaccgccaagaagtccagactgaccgacgtgaccctg

SEQ ID NO: 49 - DNA, 117 bp, Artificial sequence
  Synthetic construct
  misc_feature: CTLA4
agcctgagcaagatgctgaagaagcggagccccctgaccaccggcgtgtacgtgaagatgcctcccaccgagcccgagtgcgagaagcagttccagccctacttcatccccatcaac

SEQ ID NO: 50 - DNA, 120 bp, Artificial sequence
  Synthetic construct
  misc_feature: CD7
gccagaacccagatcaagaagctgtgcagctggcgggacaagaacagcgccgcctgtgtggtgtacgaggacatgagccacagccggtgcaacaccctgagcagccccaaccagtaccag

SEQ ID NO: 51 - DNA, 126 bp, Artificial sequence
  Synthetic construct
  misc_feature: 4-1BB
aagcggggcagaaagaagctgctgtacatcttcaagcagcccttcatgcggcccgtgcagaccacccaggaagaggacggctgctcctgcagattccccgaggaagaagaaggcggctgcgagctg

SEQ ID NO: 52 - DNA, 129 bp, Artificial sequence
  Synthetic construct
  misc_feature: CD28
tgggtgcgcagcaagagaagcagactgctgcacagcgactacatgaacatgacccccagacggcctggccccaccagaaagcactaccagccttacgcccctcccagagacttcgccgcctacagatct

SEQ ID NO: 53 - DNA, 147 bp, Artificial sequence
  Synthetic construct
  misc_feature: CD27
caccagcggcggaagtacagaagcaacaagggcgagagccccgtggaacctgccgagccttgtcactacagctgccccagagaggaagagggcagcaccatcccaatccaggaagattaccggaagcccgagcccgcctgtagccct

SEQ ID NO: 54 - DNA, 153 bp, Artificial sequence
  Synthetic construct
  misc_feature: LAG3
agaaggcagtggcggcccagaagattcagcgccctggaacagggcatccaccctccacaggcccagagcaagatcgaggaactggaacaggaacccgagcccgagcctgagccagaacctgaaccagagcccgaacccgaacctgaacagctg

SEQ ID NO: 55 - DNA, 162 bp, Artificial sequence
  Synthetic construct
  misc_feature: GITR
cacatctggcagctgcggagccagtgcatgtggcccagagagacacagctgctgctggaagtgccccccagcaccgaggatgccagaagctgccagttccccgaggaagagagaggcgagagatccgccgaggaaaagggcagactgggcgacctgtgggtg

SEQ ID NO: 56 - DNA, 183 bp, Artificial sequence
  Synthetic construct
  misc_feature: CD226
aacagacggcggagaagagagcggcgggacctgtttaccgagagctgggatacccagaaggcccccaacaactacagaagccccatcagcaccagccagcccaccaaccagagcatggacgacacccgcgaggacatctacgtgaactaccccaccttcagcagacggcccaagaccagagtg

SEQ ID NO: 57 - DNA, 186 bp, Artificial sequence
  Synthetic construct
  misc_feature: CD40
aagaaggtggccaagaagcccaccaacaaggccccccaccccaagcaggaaccccaggaaatcaacttccccgacgacctgcccggcagcaatactgctgctcccgtgcaggaaaccctgcacggctgtcagcctgtgacccaggaagatggcaaagaaagccggatctctgtgcaggaacggcag

SEQ ID NO: 58 - DNA, 231 bp, Artificial sequence
  Synthetic construct
  misc_feature: TIM-3
aagtggtacagccacagcaaagagaagatccagaacctgagcctgatcagcctggccaacctgcctcctagcggactggctaatgccgtggccgagggcatcagaagcgaggaaaacatctacaccatcgaagagaacgtgtacgaggtggaagaacccaacgagtactactgctacgtgtccagccggcagcagccctctcagcctctgggctgcagattcgccatgccc

SEQ ID NO: 59 - DNA, 240 bp, Artificial sequence
  Synthetic construct
  misc_feature: TIGIT
cggaagaagaaggccctgagaatccacagcgtggaaggcgacctgcggagaaagtctgccggccaggaagagtggtcccctagcgctccaagccctcctggctcttgtgtgcaggccgaagctgctcctgctggcctgtgtggcgaacagagaggcgaggattgcgccgagctgcacgactacttcaacgtgctgagctacagaagcctgggcaactgcagcttcttcaccgagacaggc

SEQ ID NO: 60 - DNA, 249 bp, Artificial sequence
  Synthetic construct
  misc_feature: CRTAM
aagctgagaaaggcccacgtgatctggaagaaagaaaacgaggtgtccgagcacaccctggaaagctacagaagcagaagcaacaacgaggaaaccagcagcgaggaaaagaacggccagagcagccaccccatgcggtgcatgaactacatcaccaagctgtactccgaggccaagaccaagcggaaagaaaatgtgcagcacagcaagctggaagagaagcacatccaggtgcccgagagcatcgtg

SEQ ID NO: 61 - DNA, 294 bp, Artificial sequence
  Synthetic construct
  misc_feature: PDCD1
atctgcagcagagccgccagaggcacaatcggcgccagaagaacaggccagcccctgaaagaggacccctctgccgtgcctgtgttcagcgtggactacggcgagctggacttccagtggcgggaaaagacccccgagcctcctgtgccttgtgtgcctgagcagaccgagtacgccaccatcgtgttccctagcggcatgggcacaagcagccctgccagaagaggatctgccgacggccctagatctgcccagcctctgaggcctgaggatggccactgtagctggcctctg

SEQ ID NO: 62 - DNA, 294 bp, Artificial sequence
  Synthetic construct
  misc_feature: TNFRSF25 nt Peptide
acctacagacactgctggccccacaagcccctcgtgacagccgatgaggccggaatggaagccctgacacctcctccagccacccacctgagccctctggattctgcccataccctgctggctccccccgacagcagcgagaagatctgtaccgtgcagctcgtgggcaacagctggacccctggctaccccgaaacccaggaagcactgtgtccgcaagtgacctggtcctgggaccagctgccttctagagccctgggacctgccgctgcccctacactgtctccagagtct

SEQ ID NO: 63 - DNA, 303 bp, Artificial sequence
  Synthetic construct
  misc_feature: LAIR1
caccggcagaaccagatcaagcagggccctcccagaagcaaggacgaggaacagaagccccagcagaggcccgatctggccgtggacgtgctggaaagaaccgccgataaggccaccgtgaacggcctgcccgagaaggacagagagacagacacaagcgccctggccgctggcagctctcaggaagtgacatacgcccagctggaccactgggccctgacccagagaacagccagagccgtgtctccccagagcaccaagcctatggccgagagcatcacctatgccgccgtggccagacac

SEQ ID NO: 64 - DNA, 321 bp, Artificial sequence
  Synthetic construct
  misc_feature: CXAR
tgctgccggaagaagcgccgcgaagagaagtacgagaaagaggtgcaccacgacatccgcgaggacgtgccaccccctaagagcagaaccagcaccgccagaagctacatcggcagcaaccacagcagcctgggcagcatgagccccagcaacatggaaggctacagcaagacccagtacaaccaggtgcccagcgaggacttcgagagaacccctcagagccctaccctgccccctgccaaagtggccgctcccaacctgtctagaatgggcgccatccccgtgatgatccccgcccagtctaaggacggcagcatcgtg

SEQ ID NO: 65 - DNA, 342 bp, Artificial sequence
  Synthetic construct
  misc_feature: BTLA
tgctgcctgagaaggcaccagggcaagcagaacgagctgagcgataccgccggcagagaaatcaacctggtggacgcccacctgaagtccgagcagacagaggccagcacccggcagaatagccaggtgctgctgagcgagacaggcatctacgacaacgaccccgacctgtgcttcagaatgcaggaaggcagcgaggtgtacagcaacccctgcctggaagagaacaagcccggcatcgtgtacgccagcctgaaccactctgtgatcggccccaacagcagactggcccggaacgtgaaagaggcccctaccgagtacgcctccatctgcgtgcggtct

SEQ ID NO: 66 - DNA, 351 bp, Artificial sequence
  Synthetic construct
  misc_feature: CD2
accaagcggaagaagcagcggagcagacggaacgacgaggaactggaaaccagagcccacagagtggccaccgaggaaagaggcagaaagccccaccagatccccgccagcacccctcagaatcctgccacctctcagcaccctccacccccacctggacacagatctcaggcccctagccacagacctccacctcccggacacagagtgcagcaccagcctcagaaaagaccccctgcccctagcggcacacaggtgcaccagcagaaaggccccccactgcccagacctagggtgcagcctaaacctcctcacggcgctgccgagaacagcctgagccctagcagcaac

SEQ ID NO: 67 - DNA, 360 bp, Artificial sequence
  Synthetic construct
  misc_feature: 2B4
tggaggcgcaagcggaaagagaagcagagcgagacaagccccaaagagttcctgaccatctacgaggacgtgaaggacctgaaaacccggcggaaccacgagcaggaacagacctttcctggcggcggaagcaccatctacagcatgatccagagccagagcagcgcccccacctctcaggaacctgcctacaccctgtactccctgatccagcccagcagaaagagcggcagccggaagagaaaccacagccccagcttcaacagcacaatctacgaagtgatcggcaagagccagcccaaggcccagaaccctgcccggctgagcagaaaagagctggaaaacttcgacgtgtacagc

SEQ ID NO: 68 - DNA, 561 bp, Artificial sequence
  Synthetic construct
  misc_feature: CD30
agacgggcctgccggaagcggatcagacagaaactgcacctgtgctaccccgtgcagaccagccagcccaagctggaactggtggacagccggcccagaagaagcagcacccagctgagatctggcgcctccgtgacagaacccgtggccgaagaaaggggcctgatgagccagcccctgatggaaacctgccactctgtgggagccgcctacctggaaagtctgccactgcaggatgccagccctgccggcggaccaagctctcctagagatctgcccgagcccagagtgtccaccgagcacaccaacaacaagatcgagaagatctacatcatgaaggccgacaccgtgatcgtgggcaccgtgaaagccgagctgcctgagggaagaggactggccggacctgccgagcctgagctggaagaagaactggaagccgaccacaccccccactaccccgagcaggaaacagagcctcctctgggcagctgcagcgacgtgatgctgtccgtggaagaggaaggcaaagaggaccccctgcctacagccgcctctggcaaa

SEQ ID NO: 69 - DNA, 153 bp, Artificial sequence
  Synthetic construct
  misc_feature: NKG2D
atgggctggatcagaggcagaagaagccggcacagctgggagatgagcgagttccacaactacaacctggacctgaagaagtccgacttcagcacccggtggcagaaacagagatgccccgtcgtgaagtccaagtgcagagagaacgccagc

SEQ ID NO: 70 - DNA, 144 bp, Artificial sequence
  Synthetic construct
  misc_feature: TIM-1
aagaagtactttttcaagaaagaggtgcagcagctgagcgtgtccttcagcagcctgcagatcaaggccctgcagaacgccgtggaaaaagaagtgcaggccgaggacaacatctacatcgagaacagcctgtacgccaccgac

SEQ ID NO: 71 - DNA, 255 bp, Artificial sequence
  Synthetic construct
  misc_feature: BAFF-R
tcttggcggcggagacagagaaggctgagaggcgcttcttctgccgaggcccccgacggcgataaggatgctcctgagcccctggacaaagtgatcatcctgtcccccggcatcagcgacgccacagctcctgcttggcctccacctggcgaagatcctggcacaacacctccaggccacagcgtgccagtgcctgccacagagctgggcagcacagaactcgtgaccaccaagacagccggccctgagcagcag

SEQ ID NO: 72 - DNA, 333 bp, Artificial sequence
  Synthetic construct
  misc_feature: TACI
gcctgcttcctgaagaagagaggcgacccctgcagctgccagcccagaagcagacctagacagagccccgccaagagcagccaggaccatgccatggaagccggcagccctgtgtctacctctcccgagcctgtggaaacctgcagcttctgcttccccgagtgcagagcccccacccaggaatctgctgtgacccctggcacccccgatcctacatgtgccggcagatggggctgccacaccagaaccacagtgctgcagccctgcccccacatccctgattctggcctgggcatcgtgtgtgtgcctgcccaggaaggcggacctggcgct

SEQ ID NO: 73 - DNA, 321 bp, Artificial sequence
  Synthetic construct
  misc_feature: BCMAI
cggaagatcaacagcgagcccctgaaggacgagttcaagaacaccggctctggcctgctgggcatggccaacatcgacctggaaaagagccggaccggcgacgagatcatcctgcctagaggcctggagtacaccgtggaagagtgtacctgcgaggactgcatcaagagcaagcccaaggtggacagcgaccactgcttccccctgcccgctatggaagagggcgccacaatcctcgtgaccaccaagaccaacgactactgcaagagcctgcctgccgccctgagcgccaccgagatcgagaagtctatcagcgcccgg

SEQ ID NO: 74 - DNA, 285 bp, Artificial sequence
  Synthetic construct
  misc_feature: CD72
atggccgaggccatcacctacgccgacctgagattcgtgaaggcccccctgaagaagtccatcagcagcagactgggccaggaccctggcgccgatgatgatggcgagatcacatacgagaacgtgcaggtgccagccgtgctgggagtgccatcttctctggccagtagcgtgctgggcgataaggccgccgtgaagtctgagcagcctaccgcctcttggagagccgtgacatctcctgccgtgggcagaatcctgccctgcagaaccacctgtctgcggtac

SEQ ID NO: 75 - DNA, 135 bp, Artificial sequence
  Synthetic construct
  misc_feature: CD96
agaaagtggtgccagtaccagaaagaaatcatggaacggcccccacccttcaagccccctccaccccctatcaagtacacctgtatccaggaacccaacgagagcgacctgccctaccacgagatggaaaccctg

SEQ ID NO: 76 - DNA, 252 bp, Artificial sequence
  Synthetic construct
  misc_feature: NTB-A
ctgcggaagagaagagacagcctgagcctgagcacccagagaacacagggacctgccgagagcgcccggaacctggaatacgtgtccgtgtcccccaccaacaacaccgtgtacgccagcgtgacccacagcaacagagagacagagatttggacccccagagagaacgacaccatcaccatctacagcaccatcaaccactccaaagagagcaagcccaccttcagcagagccaccgccctggacaatgtg

SEQ ID NO: 77 - DNA, 264 bp, Artificial sequence
  Synthetic construct
  misc_feature: CRACC
tggttcctgaagcgcgagcggcaggaagagtacatcgaagagaagaaacgggtggacatctgcagagagacacccaacatctgcccccacagcggcgagaacaccgagtacgacaccatcccccacaccaaccggaccatcctgaaagaggaccccgccaacaccgtgtacagcaccgtggaaatccccaagaagatggaaaacccccactccctgctgaccatgcccgacacccctagactgttcgcctacgagaacgtgatc

SEQ ID NO: 78 - DNA, 246 bp, Artificial sequence
  Synthetic construct
  misc_feature: Siglec-3.9.9
aagacccacagacggaaggccgccagaacagccgtgggcagaaacgacacccaccctacaacaggcagcgccagccccaagcaccagaagaagtctaagctgcacggccccaccgagacaagcagctgtagcggagctgcccccaccgtggaaatggacgaggaactgcactacgccagcctgaacttccacggcatgaaccccagcaaggacaccagcaccgagtacagcgaagtgcggacccag

SEQ ID NO: 79 - DNA, 114 bp, Artificial sequence
  Synthetic construct
  misc_feature: KLRG1
atgaccgacagcgtgatctactccatgctggaactgcccaccgccacccaggcccagaatgattatggcccccagcagaagtccagcagcagcagacctagctgctcctgcctg

SEQ ID NO: 80 - DNA, 135 bp, Artificial sequence
  Synthetic construct
  misc_feature: NKR-P1A
atggaccagcaggccatctacgccgagctgaacctgcccacagatagcggccctgagagcagcagccctagcagcctgcctagagatgtgtgccagggctccccctggcaccagtttgccctgaagctgagctgc

SEQ ID NO: 81 - DNA, 504 bp, Artificial sequence
  Synthetic construct
  misc_feature: ILT2
ctgagacacagacggcagggcaagcactggaccagcacccagagaaaggccgacttccagcatcctgccggcgctgtgggacctgagcctacagatagaggactgcagtggcggtctagccctgccgccgatgcccaggaagagaatctgtacgccgccgtgaagcacacccagcccgaagatggcgtggaaatggacaccagaagcccccacgacgaggaccctcaggccgtgacatacgccgaagtgaaacacagccggcctcggagagagatggccagccctccatctcctctgagcggcgagttcctggacaccaaggacagacaggccgaagaggaccggcagatggatacagaggccgctgcttctgaggccccccaggatgtgacatatgcccagctgcacagcctgaccctgcggagagaagccacagagcctcccccaagccaggaaggaccttctcctgccgtgccttccatctacgccaccctggccattcac

SEQ ID NO: 82 - DNA, 249 bp, Artificial sequence
  Synthetic construct
  misc_feature: KIR2DL1
agatggtgcagcaacaagaaaaacgccgccgtgatggaccaggaaagcgccggcaacagaaccgccaacagcgaggatagcgacgagcaggacccccaggaagtgacctacacccagctgaaccactgcgtgttcacccagcggaagatcaccagacccagccagaggcccaagaccccccccaccgatatcatcgtgtacaccgagctgcccaacgccgagagcagatccaaggtggtgtcctgccct

SEQ ID NO: 83 - DNA, 252 bp, Artificial sequence
  Synthetic construct
  misc_feature: KIR3DL1
cacctgtggtgcagcaacaagaaaaacgccgccgtgatggaccaggaacctgccggcaacagaaccgccaacagcgaggatagcgacgagcaggaccccgaggaagtgacctacgcccagctggaccactgcgtgttcacccagcggaagatcaccagacccagccagaggcccaagaccccccctaccgataccatcctgtacaccgagctgcccaacgccaagcccagatccaaggtggtgtcctgccct

SEQ ID NO: 84 - DNA, 504 bp, Artificial sequence
  Synthetic construct
  misc_feature: ILT3
cagcattggagacagggcaagcacagaaccctggcccagagacaggccgacttccagaggcctcctggcgccgctgagcctgagcctaaagatggcggcctgcagcggagaagctctcctgcagctgatgtgcagggcgagaacttctgtgccgccgtgaagaacacccagcccgaggacggcgtggaaatggacaccagacagagcccccacgacgaggaccctcaggccgtgacatacgccaaagtgaagcacagccggcctcggagagagatggccagccctccatctcctctgagcggcgagttcctggacaccaaggacaggcaggccgaagaggaccggcagatggatacagaggccgctgcttctgaggccccccaggatgtgacatatgcccagctgcacagcttcaccctgcggcagaaggccacagagcctcccccaagccaggaaggcgcttctcctgccgagcctagcgtgtacgccacactggccattcac

SEQ ID NO: 85 - DNA, 348 bp, Artificial sequence
  Synthetic construct
  misc_feature: ILT4
ctgagacacagacggcagggcaagcactggaccagcacccagagaaaggccgacttccagcatcctgccggcgctgtgggacctgagcctacagatagaggactgcagtggcggtctagccctgccgccgatgcccaggaagagaatctgtacgccgccgtgaaggacacccagcccgaagatggcgtggaaatggacaccagagccgccgctagcgaggcccctcaggatgtgacatatgcccagctgcacagcctgaccctgcggaggaaagccacagagcctcccccaagccaggaaagagagcctcctgccgagcccagcatctacgccacactggccattcac

SEQ ID NO: 86 - DNA, 384 bp, Artificial sequence
  Synthetic construct
  misc_feature: TLT-1
atggccaagcggaagcagggcaacagactgggcgtgtgcggcagattcctgagcagcagagtgtccggcatgaaccccagcagcgtggtgcatcacgtgtccgattctggacctgccgccgagctgcctctggatgtgcctcacatcagactggacagcccccccagcttcgacaacaccacctacaccagcctgcccctggatagccctagcggcaagccttctctgcctgccccaagctctctgccccctctgcctccaaaagtgctcgtgtgcagcaagcccgtgacctacgccaccgtgatcttccctggcggcaacaagggcggaggcacaagctgtggacccgcccagaacccccccaacaatcagacacctagcagc

SEQ ID NO: 87 - DNA, 183 bp, Artificial sequence
  Synthetic construct
  misc_feature: CD200R
aaagtgaacggctgccggaagtacaagctgaacaagaccgagagcacccccgtggtggaagaggacgagatgcagccctacgccagctacaccgagaagaacaaccccctgtacgacaccaccaacaaagtgaaggccagcgaggccctgcagagcgaggtggacaccgatctgcacaccctg

SEQ ID NO: 88 - DNA, 294 bp, Artificial sequence
  Synthetic construct
  misc_feature: CD300a
cggatgttccagaagtggatcaaggccggcgaccacagcgagctgagccagaatcctaagcaggccgccacccagagcgagctgcactacgccaatctggaactgctgatgtggccactgcaggaaaagcctgcccccccacgggaagtggaagtggaatactccaccgtggccagccccagagaggaactgcattacgccagcgtggtgttcgacagcaacaccaacagaatcgccgcccagcggcccagagaagaggaacccgatagcgactacagcgtgatcagaaagacc

SEQ ID NO: 89 - DNA, 339 bp, Artificial sequence
  Synthetic construct
  misc_feature: CD300f
tggcggatgatgaagtaccagcagaaagccgccggaatgagccccgaacaggtgctgcagcctctggaaggcgatctgtgctacgccgacctgacactgcagctggccggcacatctcctcagaaggccaccaccaagctgagcagcgctcaggtggaccaggtggaagtggaatacgtgaccatggccagcctgcccaaagaggacatcagctacgccagcctgaccctgggagccgaggatcaggaacccacctactgcaacatgggccacctgtccagccatctgcctggcagaggacccgaggaacctaccgagtacagcaccatcagccggccc

SEQ ID NO: 90 - DNA, 111 bp, Artificial sequence
  Synthetic construct
  misc_feature: DC-SIGN
atgagcgacagcaaagagccccggctgcagcagctgggactgctggaagaggaacagctgagaggcctgggcttccggcagaccagaggctacaaaagcctggccggctgc

SEQ ID NO: 91 - DNA, 285 bp, Artificial sequence
  Synthetic construct
  misc_feature: Allergin-1
ctgcccaagtacaagacccggaaggccatgcggaacaacgtgcccagagacagaggcgacaccgccatggaagtgggcatctacgccaacatcctggaaaagcaggccaaagaggaatccgtgcccgaagtgggctccagaccttgtgtgtccaccgcccaggatgaggccaagcacagccaggaactgcagtacgccacccccgtgttccaggaagtggcccccagagaacaggaagcctgcgacagctacaagagcggctacgtgtacagcgagctgaacttc

SEQ ID NO: 92 - DNA, 534 bp, Artificial sequence
  Synthetic construct
  misc_feature: PIR-B
cggagaaggcaccggggcaagttcagaaaggacgtgcagaaagagaaggacctgcagctgagcagcggagccgaggaacccatcaccagaaagggcgagctgcagaagaggcccaatcctgccgccgctacccaggaagagtctctgtacgccagcgtggaagatatgcagaccgaggacggcgtggaactgaacagctggaccccccctgaggaagatccccagggcgagacatacgcccaagtgaagcccagcagactgcggaaggccggccatgtgtctcccagcgtgatgagcagagagcagctgaacaccgagtacgagcaggccgaggaaggacagggcgccaacaatcaggccgccgagtctggcgagagccaggatgtgacatatgcccagctgtgcagccggaccctgagacagggggctgctgcttctccactgagccaggctggcgaggccccagaggaaccttctgtgtatgccaccctggccgctgccagacctgaggccgtgcccaaggatatggaacag

SEQ ID NO: 93 - DNA, 126 bp, Artificial sequence
  Synthetic construct
  misc_feature: 4-1BB.wt
aaacggggcagaaagaaactcctgtatatattcaaacaaccatttatgaggccagtacaaactactcaagaggaagatggctgtagctgccgatttccagaagaagaagaaggaggatgtgaactg

SEQ ID NO: 94 - DNA, 126 bp, Artificial sequence
  Synthetic construct
  misc_feature: CD28.wt
gtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 95 - DNA, 117 bp, Artificial sequence
  Synthetic construct
  misc_feature: ICOS.wt
tgctgttggcttacaaaaaagaagtattcatccagtgtgcacgaccctaacggtgaatacatgttcatgagagcagtgaacacagccaaaaaatctagactcacagatgtgacccta

SEQ ID NO: 96 - protein, 11 aa, Artificial sequence
  Synthetic construct
  MISC_FEATURE: CD8 transmembrane domain
VLLLSLVITLY

SEQ ID NO: 97 - protein, 15 aa, Artificial sequence
  Synthetic construct
  MISC_FEATURE: CD3 zeta
RVKFSRSADAPAYQQ

SEQ ID NO: 98 - DNA, 32 bp, Artificial sequence
  Synthetic construct
  misc_feature: CD8 transmembrane domain
gtccttctcctgtcactggttatcacccttta

SEQ ID NO: 99 - DNA, 45 bp, Artificial sequence
  Synthetic construct
  misc_feature: CD3 zeta
agagtgaagttcagcaggagcgcagacgcccccgcgtaccagcag

SEQ ID NO: 100 - protein, 242 aa, Artificial sequence
  Synthetic construct
  MISC_FEATURE: anti-CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 101 - DNA, 726 bp, Artificial sequence
  Synthetic construct
  misc_feature: anti-CD19 scFv
gacatccagatgacacagactacatcctccctgtctgcctctctgggtgacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtatcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtatcctca

SEQ ID NO: 102 - protein, 25 aa, Artificial sequence
  Synthetic construct
  MISC_FEATURE: B7-1 (CD80)
TYCFAPRCRERRRNERLRRESVRPV

SEQ ID NO: 103 - DNA, 75 bp, Artificial sequence
  Synthetic construct
  misc_feature: B7-1 (CD80)
acctactgctttgccccaagatgcagagagagaaggaggaatgagagattgagaagggaaagtgtacgccctgta

SEQ ID NO: 104 - protein, 285 aa, Artificial sequence
  Synthetic construct
  MISC_FEATURE: DR6 (TNFRSF21)
RKSSRTLKKGPRQDPSAIVEKAGLKKSMTPTQNREKWIYYCNGHGIDILKLVAAQVGSQWKDIYQFLCNASEREVAAFSNGYTADHERAYAALQHWTIRGPEASLAQLISALRQHRRNDVVEKIRGLMEDTTQLETDKLALPMSPSPLSPSPIPSPNAKLENSALLTVEPSPQDKNKGFFVDESEPLLRCDSTSSGSSALSRNGSFITKEKKDTVLRQVRLDPCDLQPIFDDMLHFLNPEELRVIEEIPQAEDKLDRLFEIIGVKSQEASQTLLDSVYSHLPDLL

SEQ ID NO: 105 - DNA, 855 bp, Artificial sequence
  Synthetic construct
  misc_feature: DR6 (TNFRSF21) nt Peptide
cggaaaagctcgaggactctgaaaaaggggccccggcaggatcccagtgccattgtggaaaaggcagggctgaagaaatccatgactccaacccagaaccgggagaaatggatctactactgcaatggccatggtatcgatatcctgaagcttgtagcagcccaagtgggaagccagtggaaagatatctatcagtttctttgcaatgccagtgagagggaggttgctgctttctccaatgggtacacagccgaccacgagcgggcctacgcagctctgcagcactggaccatccggggccccgaggccagcctcgcccagctaattagcgccctgcgccagcaccggagaaacgatgttgtggagaagattcgtgggctgatggaagacaccacccagctggaaactgacaaactagctctcccgatgagccccagcccgcttagcccgagccccatccccagccccaacgcgaaacttgagaattccgctctcctgacggtggagccttccccacaggacaagaacaagggcttcttcgtggatgagtcggagccccttctccgctgtgactctacatccagcggctcctccgcgctgagcaggaacggttcctttattaccaaagaaaagaaggacacagtgttgcggcaggtacgcctggacccctgtgacttgcagcctatctttgatgacatgctccactttctaaatcctgaggagctgcgggtgattgaagagattccccaggctgaggacaaactagaccggctattcgaaattattggagtcaagagccaggaagccagccagaccctcctggactctgtttatagccatcttcctgacctgctg

SEQ ID NO: 106 - protein, 179 aa, Artificial sequence
  Synthetic construct
  MISC_FEATURE: LY9
KRKGRCSVPAFCSSQAEAPADTPEPTAGHTLYSVLSQGYEKLDTPLRPARQQPTPTSDSSSDSNLTTEEDEDRPEVHKPISGRYEVFDQVTQEGAGHDPAPEGQADYDPVTPYVTEVESVVGENTMYAQVFNLQGKTPVSQKEESSATIYCSIRKPQVVPPPQQNDLEIPESPTYENFT

SEQ ID NO: 107 - DNA, 537 bp, Artificial sequence
  Synthetic construct
  misc_feature: LY9
aagcgaaaaggacggtgttcagtcccagccttctgttccagccaagctgaggccccagcggatacaccagaacccacagctggccacacgctatactctgtgctctcccaaggatatgagaagctggacactcccctcaggcctgccaggcaacagcctacacccacctcagacagcagctctgacagcaacctcacaactgaggaggatgaggacaggcctgaggtgcacaagcccatcagtggaagatatgaggtatttgaccaggtcactcaggagggcgctggacatgacccagcccctgagggccaagcagactatgatcccgtcactccatatgtcacggaagttgagtctgtggttggagagaacaccatgtatgcacaagtgttcaacttacagggaaagaccccagtttctcagaaggaagagagctcagccacaatctactgctccatacggaaacctcaggtggtgccaccaccacaacagaatgatcttgagattcctgaaagtcctacctatgaaaatttcacc

SEQ ID NO: 108 - protein, 155 aa, Artificial sequence
  Synthetic construct
  MISC_FEATURE: NGFR
KRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 109 - DNA, 465 bp, Artificial sequence
  Synthetic construct
  misc_feature: NGFR
aagaggtggaacagctgcaagcagaacaagcaaggagccaacagccggccagtgaaccagacgcccccaccagagggagaaaaactccacagcgacagtggcatctccgtggacagccagagcctgcatgaccagcagccccacacgcagacagcctcgggccaggccctcaagggtgacggaggcctctacagcagcctgcccccagccaagcgggaggaggtggagaagcttctcaacggctctgcgggggacacctggcggcacctggcgggcgagctgggctaccagcccgagcacatagactcctttacccatgaggcctgccccgttcgcgccctgcttgcaagctgggccacccaggacagcgccacactggacgccctcctggccgccctgcgccgcatccagcgagccgacctcgtggagagtctgtgcagtgagtccactgccacatccccggtg

SEQ ID NO: 110 - protein, 42 aa, Artificial sequence
  Synthetic construct
  MISC_FEATURE: OX40
ALYLLRRDQRLPPDAHKPPGGGSFRTPIQEEQADAHSTLAKI

SEQ ID NO: 111 - DNA, 126 bp, Artificial sequence
  Synthetic construct
  misc_feature: OX40
gccctgtacctgctccggagggaccagaggctgccccccgatgcccacaagccccctgggggaggcagtttccggacccccatccaagaggagcaggccgacgcccactccaccctggccaagatc

SEQ ID NO: 112 - protein, 247 aa, Artificial sequence
  Synthetic construct
  MISC_FEATURE: RELT
NLLKRKGYHCTAHKEVGPGPGGGGSGINPAYRTEDANEDTIGVLVRLITEKKENAAALEELLKEYHSKQLVQTSHRPVSKLPPAPPNVPHICPHRHHLHTVQGLASLSGPCCSRCSQKKWPEVLLSPEAVAATTPVPSLLPNPTRVPKAGAKAGRQGEITILSVGRFRVARIPEQRTSSMVSEVKTITEAGPSWGDLPDSPQPGLPPEQQALLGSGGSRTKWLKPPAENKAEENRYVVRLSESNLVI

SEQ ID NO: 113 - DNA, 741 bp, Artificial sequence
  Synthetic construct
  misc_feature: RELT
aacctcctcaagcggaagggctaccactgcacggcgcacaaggaggtcgggcccggccctggaggtggaggcagtggaatcaaccctgcctaccggactgaggatgccaatgaggacaccattggggtcctggtgcgcttgatcacagagaagaaagagaatgctgcggccctggaggagctgctgaaagagtaccacagcaaacagctggtgcagacgagccacaggcctgtgtccaagctgccgccagcgcccccgaacgtgccacacatctgcccgcaccgccaccatctccacaccgtgcagggcctggcctcgctctctggcccctgctgctcccgctgtagccagaagaagtggcccgaggtgctgctgtcccctgaggctgtagccgccactactcctgttcccagccttctgcctaacccgaccagggttcccaaggccggggccaaggcagggcgtcagggcgagatcaccatcttgtctgtgggcaggttccgcgtggctcgaattcctgagcagcggacaagttcaatggtgtctgaggtgaagaccatcacggaggctgggccctcgtggggtgatctccctgactccccacagcctggcctcccccctgagcagcaggccctgctaggaagtggcggaagccgtacaaagtggctgaagcccccagcagagaacaaggccgaggagaaccgctatgtggtccggctaagtgagagcaacctggtcatc

SEQ ID NO: 114 - protein, 232 aa, Artificial sequence
  Synthetic construct
  MISC_FEATURE: TROY
YCKRQFMEKKPSWSLRSQDIQYNGSELSCFDRPQLHEYAHRACCQCRRDSVQTCGPVRLLPSMCCEEACSPNPATLGCGVHSAASLQARNAGPAGEMVPTFFGSLTQSICGEFSDAWPLMQNPMGGDNISFCDSYPELTGEDIHSLNPELESSTSLDSNSSQDLVGGAVPVQSHSENFTAATDLSRYNNTLVESASTQDALTMRSQLDQESGAVIHPATQTSLQVRQRLGSL

SEQ ID NO: 115 - DNA, 696 bp, Artificial sequence
  Synthetic construct
  misc_feature: TROY
tattgtaagagacagtttatggagaagaaacccagctggtctctgcggtcacaggacattcagtacaacggctctgagctgtcgtgttttgacagacctcagctccacgaatatgcccacagagcctgctgccagtgccgccgtgactcagtgcagacctgcgggccggtgcgcttgctcccatccatgtgctgtgaggaggcctgcagccccaacccggcgactcttggttgtggggtgcattctgcagccagtcttcaggcaagaaacgcaggcccagccggggagatggtgccgactttcttcggatccctcacgcagtccatctgtggcgagttttcagatgcctggcctctgatgcagaatcccatgggtggtgacaacatctctttttgtgactcttatcctgaactcactggagaagacattcattctctcaatccagaacttgaaagctcaacgtctttggattcaaatagcagtcaagatttggttggtggggctgttccagtccagtctcattctgaaaactttacagcagctactgatttatctagatataacaacacactggtagaatcagcatcaactcaggatgcactaactatgagaagccagctagatcaggagagtggtgctgtcatccacccagccactcagacgtccctccaggtaaggcagcgactgggttccctg

SEQ ID NO: 116 - protein, 61 aa, Artificial sequence
  Synthetic construct
  MISC_FEATURE: B7-2 (CD86)
KWKKKKRPRNSYKCGTNTMEREESEQTKKREKIHIPERSDEAQRVFKSSKTSSCDKSDTCF

SEQ ID NO: 117 - DNA, 183 bp, Artificial sequence
  Synthetic construct
  misc_feature: B7-2 (CD86)
aaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttt

SEQ ID NO: 118 - protein, 120 aa, Artificial sequence; identical to SEQ ID NO: 20
  Synthetic construct
  MISC_FEATURE: CD244

SEQ ID NO: 119 - DNA, 360 bp, Artificial sequence
  Synthetic construct
  misc_feature: CD244
tggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattcc

SEQ ID NO: 120 - protein, 77 aa, Artificial sequence; identical to SEQ ID NO: 11
  Synthetic construct
  MISC_FEATURE: HAVCR2

SEQ ID NO: 121 - DNA, 231 bp, Artificial sequence
  Synthetic construct
  misc_feature: HAVCR2
aaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgcca